(19) United States
(12) Patent Application Publication
(10) Pub. No.: US 2026/0356823 A1
(43) Pub. Date: Nov. 12, 2026

(54) SODIUM CHANNEL REGULATOR AND USE THEREOF

(71) Applicant: SHANGHAI HUILUN PHARMACEUTICAL CO., LTD., Shanghai (CN)

(72) Inventors: Shudong Duan, Shanghai (CN); Shuqi Du, Shanghai (CN); Xinfeng Wang, Shanghai (CN); Lei Chen, Shanghai (CN); Jihong Qin, Shanghai (CN)

(21) Appl. No.: 19/107,567

(22) PCT Filed: Aug. 28, 2023
(86) PCT No.: PCT/CN2023/115177

§ 371 Date: Feb. 27, 2025

(30) Foreign Application Priority Data
Aug. 28, 2022 (CN) 202211036306.3
Nov. 3, 2022 (CN) 202211374105.4
Mar. 31, 2023 (CN) 202310334077.1

(51) Int. Cl. C07D 405/12; A61K 31/341; A61K 31/343; A61K 31/353; A61K 31/357; A61K 31/36; A61K 31/381; A61K 31/40; A61K 31/4025; A61K 31/403; A61K 31/4035; A61K 31/404; A61K 31/423; A61K 31/427; A61K 31/428; A61K 31/4375; A61K 31/44; A61K 31/443; A61K 31/4433; A61K 31/4436; A61K 31/4439; A61K 31/4725; A61K 31/496; A61K 31/501; A61K 31/502; A61K 31/517; A61K 31/519; A61K 31/541; A61K 31/549; A61K 31/675; A61P 29/00; C07B 59/00; C07C 237/42; C07D 207/16; C07D 209/34; C07D 209/46; C07D 209/52; C07D 213/81; C07D 307/24; C07D 307/93; C07D 307/937; C07D 333/38; C07D 333/48; C07D 401/12; C07D 403/12; C07D 405/04; C07D 405/14; C07D 407/04; C07D 409/12; C07D 413/12; C07D 413/14; C07D 417/12; C07D 417/14; C07D 471/04; C07D 487/04; C07F 9/6558

(52) U.S. Cl. CPC C07D 405/12; A61K 31/341; A61K 31/343; A61K 31/353; A61K 31/357; A61K 31/36; A61K 31/381; A61K 31/40; A61K 31/4025; A61K 31/403; A61K 31/4035; A61K 31/404; A61K 31/423; A61K 31/427; A61K 31/428; A61K 31/4375; A61K 31/44; A61K 31/443; A61K 31/4433; A61K 31/4436; A61K 31/4439; A61K 31/4725; A61K 31/496; A61K 31/501; A61K 31/502; A61K 31/517; A61K 31/519; A61K 31/541; A61K 31/549; A61K 31/675; A61P 29/00; C07B 59/002; C07C 237/42; C07D 207/16; C07D 209/34; C07D 209/46; C07D 209/52; C07D 213/81; C07D 307/24; C07D 307/93; C07D 307/937; C07D 333/38; C07D 333/48; C07D 401/12; C07D 403/12; C07D 405/04; C07D 405/14; C07D 407/04; C07D 409/12; C07D 413/12; C07D 413/14; C07D 417/12; C07D 417/14; C07D 471/04; C07D 487/04; C07F 9/65586; C07C 2601/08

(57) ABSTRACT

Provided in the present invention are a sodium channel regulator and the use thereof. The sodium channel regulator of the present invention has a brand-new structure and has an excellent in-vitro inhibitory effect on Naᵥ 1.8. The compound or a pharmaceutically acceptable salt thereof according to the present invention can be used for treating sodium channel-related diseases, including pain, multiple sclerosis, Charcot-Marie-Tooth, incontinence, pathological cough or arrhythmia.

This application claims priorities to Chinese Patent Application No. 202211036306.3 filed on Aug. 28, 2022, Chinese Patent Application No. 202211374105.4 filed on Nov. 3, 2022, and Chinese Patent Application No. 202310334077.1 filed on Mar. 31, 2023, all of which are incorporated herein by reference in their entireties.

### TECHNICAL FIELD

The present invention relates to the field of medicine, and specifically, to a sodium channel modulator, a preparation method thereof, and therapeutic use thereof in treatment of disease.

### BACKGROUND

Pain is a complex sensation, usually uncomfortable, caused by physical injury, illness, or undesirable external stimuli. For clinical research purposes, the International Association for the Study of Pain (IASP) defines pain as “an unpleasant sensory and emotional experience associated with actual or potential tissue damage”. Pain serves as a warning signal that alerts the body to potential danger, which is indispensable for the protection of normal life activities. Pain is also a common clinical symptom. After the disappearance of an external stimulus that causes pain, the intense or persistent pain can cause physiological dysfunctions, which seriously affect the quality of life of living organisms. Some data shows that about one in five people worldwide suffer from moderate or severe chronic pain.

Pain originates in nociceptors of the peripheral nervous system. The nociceptors are free nerve endings that are widely distributed throughout the body in the skin, muscles, joints, and visceral tissues, and that can convert perceived thermal, mechanical, or chemical stimuli into nerve impulses (action potentials) and transmit the nerve impulses via afferent nerve fibers to their cell bodies located in dorsal root ganglia (DRG), and ultimately to higher nervous centers to elicit pain perception. The generation and conduction of action potentials in neurons depend on voltage-gated sodium channels (VGSCs) on cell membranes. When the cell membrane is depolarized, the sodium channels are activated and open, allowing sodium ions to flow inward, which further depolarizes the cell membrane, leading to the generation of action potentials. Therefore, the inhibition of abnormal activity of sodium channels contributes to the treatment and relief of pain.

Voltage-gated sodium channels are widely found on cell membranes of excitable cells such as neurons and skeletal muscle cells, and are a class of transmembrane glycoprotein complexes composed of one α subunit and several β subunits. The α subunit is a functional carrier of the sodium channels and consists of 1700-2000 amino acids. The β subunit primarily plays an auxiliary role in the modification of kinetics and voltage gating dependence of the ion channels. Sodium channels may be categorized based on different a subunits. Currently, nine sodium channel isoforms, Naᵥ1 (Naᵥ1.1 to Naᵥ1.9), have been identified in mammals. Different isoforms exhibit different tissue distributions, and different electrophysiological and pharmacological characteristics. Based on whether they can be effectively inhibited by nanomolar tetrodotoxin (TTX), sodium channels are categorized into TTX-sensitive (TTX-S) sodium channels and TTX-resistant (TTX-R) sodium channels. Naᵥ1.1, Naᵥ1.2, Naᵥ1.3, and Naᵥ1.7 are TTX-S, with encoding genes located on human chromosome 2q23-24, and abundantly expressed in neurons. Naᵥ1.5, Naᵥ1.8, and Naᵥ1.9 are TTX-R, with encoding genes located on human chromosome 3p21-24. Naᵥ1.5 is found primarily in cardiomyocytes. Naᵥ1.8 and Naᵥ1.9 are found in the peripheral nervous system (PNS).

Naᵥ1.8 is an important ion channel involved in chronic pain, atrial fibrillation, and Budd-Chiari syndrome, and is a highly selective target for the treatment of pain. It has been proved that Naᵥ1.8 as a carrier of sodium currents maintains action potential firing of small dorsal root ganglion neurons, and is also involved in spontaneous electrical signaling of damaged neurons, for example, driving the production of neuropathic pain. Currently reported small-molecule Naᵥ1.8 inhibitors include PF-01247324, A-803467, PF-06305591, VX-150, HRS-4800, JMKX-000623, HBW-004, VX-548, and the like. PF-01247324, A-803467, and PF-06305591 have the disadvantages of poor selectivity, poor pharmacokinetic data, and low bioavailability.

### SUMMARY

According to a first aspect of the present invention, provided is a compound shown in formula I

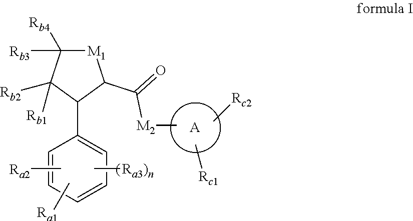

or a pharmaceutically acceptable salt thereof, where the compound further includes a stereoisomer and a racemate thereof when a chiral center is present.

In some implementations of the present invention, the compound is further shown in formula I-A or I-B:

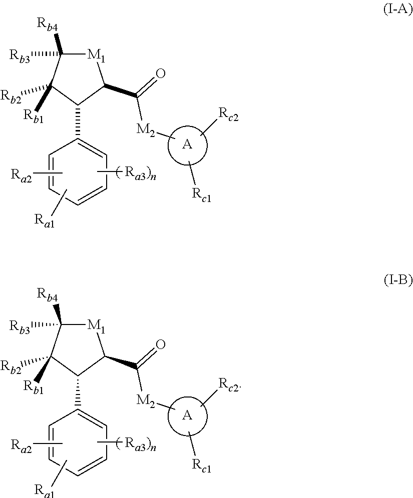

In the compound, Rₐ₁, Rₐ₂, and Rₐ₃ are each independently selected from hydrogen, halogen, hydroxyl, C₁-C₅ alkyl, C₁-C₅ alkoxy, C₃-C₆ cycloalkyl, C₃-C₆ cycloalkoxy, or —N(R_{c3}R_{c4})ₘ, and the alkyl, alkoxy, cycloalkyl, or cycloalkoxy is optionally substituted with one or more halogen, hydroxyl, cyano, C₁-C₅ alkyl, C₁-C₅ alkoxy, or amino.

In some implementations of the present invention, any adjacent two of Rₐ₁, Rₐ₂, and Rₐ₃ are linked together to form a 5- to 7-membered ring, the 5- to 7-membered ring may be a saturated or unsaturated carbocyclic or heterocyclic ring; the heterocyclic ring optionally contains one or more N, O, or S(═O)ₘ heteroatoms; and the 5- to 7-membered ring may be optionally substituted with one or more halogen, hydroxyl, cyano, C₁-C₅ alkyl, C₁-C₅ alkoxy, or amino.

In some specific implementations of the present invention, Rₐ₁, Rₐ₂, and Rₐ₃ are each independently selected from fluorine, chlorine, methyl, ethyl, methoxy, ethoxy, propoxy, trifluoromethoxy, difluoromethoxy, or amino.

In some specific implementations, any adjacent two of Rₐ₁, Rₐ₂, and Rₐ₃ are linked together to form a 5- or 6-membered carbocyclic or heterocyclic ring.

In some specific implementations, the 5- or 6-membered heterocyclic ring contains one or two N, O, or S(═O)ₘ heteroatoms.

In some specific implementations, the 5- or 6-membered carbocyclic or heterocyclic ring is optionally substituted with one or more halogen, hydroxyl, cyano, C₁-C₅ alkyl, C₁-C₅ alkoxy, or amino.

In the compound, R_{b1}, R_{b2}, R_{b3}, and R_{b4} are each independently selected from hydrogen, halogen, hydroxyl, C₁-C₅ alkyl or C₁-C₅ alkoxy, and the alkyl or alkoxy is optionally substituted with one or more halogen, hydroxyl, cyano, C₁-C₅ alkyl, C₁-C₅ alkoxy, or amino.

In a specific implementation of the present invention, R_{b1}, R_{b2}, R_{b3}, and R_{b4} are each independently selected from hydrogen, methyl, trifluoromethyl, ethyl, methoxy, or ethoxy.

In some specific implementations, R_{b1} and R_{b2} are each independently selected from hydrogen, methyl, or trifluoromethyl.

In some specific implementations, R_{b3} and R_{b4} are each independently selected from hydrogen, methyl, or trifluoromethyl.

In some specific implementations, any two of R_{b1}, R_{b2}, R_{b3}, and R_{b4} are linked together to form a 3- to 7-membered ring, the 3- to 7-membered ring may be a saturated or unsaturated carbocyclic or heterocyclic ring; the heterocyclic ring optionally contains one or more N, O, or S(═O)ₘ heteroatoms; and the 3- to 7-membered ring may be further optionally substituted with one or more halogen, hydroxyl, cyano, C₁-C₅ alkyl, C₁-C₅ alkoxy, or amino.

In some specific implementations, R_{b1} and R_{b2} are linked together to form a 3- to 7-membered saturated carbocyclic ring, and the 3- to 7-membered ring may be optionally substituted with one or more halogen, hydroxyl, cyano, C₁-C₅ alkyl, C₁-C₅ alkoxy, or amino.

In some specific implementations, R_{b1} and R_{b2} are linked together to form a 3-membered saturated carbocyclic ring, and the 3-membered ring may be optionally substituted with one or more halogen, hydroxyl, cyano, C₁-C₅ alkyl, C₁-C₅ alkoxy, or amino.

In some specific implementations, R_{b3} and R_{b4} are linked together to form a 3- to 7-membered saturated carbocyclic ring, and the 3- to 7-membered ring may be optionally substituted with one or more halogen, hydroxyl, cyano, C₁-C₅ alkyl, C₁-C₅ alkoxy, or amino.

In some specific implementations, any one of R_{b1} and R_{b2} and any one of R_{b3} and R_{b4} are linked together to form a 3- to 7-membered ring, and the 3- to 7-membered ring may be optionally substituted with one or more halogen, hydroxyl, cyano, C₁-C₅ alkyl, C₁-C₅ alkoxy, or amino.

In some specific implementations, any one of R_{b1} and R_{b2} and any one of R_{b3} and R_{b4} are linked together to form a 3- to 7-membered saturated carbocyclic ring, and the 3- to 7-membered saturated carbocyclic ring may be optionally substituted with one or more halogen, hydroxyl, cyano, C₁-C₅ alkyl, C₁-C₅ alkoxy, or amino.

In some specific implementations, any one of R_{b1} and R_{b2} and any one of R_{b3} and R_{b4}are linked together to form a 3- to 6-membered saturated carbocyclic ring, and the 3- to 6-membered saturated carbocyclic ring may be optionally substituted with one or more halogen, hydroxyl, cyano, C₁-C₅ alkyl, C₁-C₅ alkoxy, or amino.

In the present invention, a linkage between any one of R_{b1} and R_{b2} and any one of R_{b3} and R_{b4} may be a linkage between R_{b1} and R_{b3}, a linkage between R_{b1} and R_{b4}, a linkage between R_{b2} and R_{b3}, or a linkage between R_{b2} and R_{b4}.

M₁ and M₂ are each independently selected from C, O, S(═O)ₘ, or N—Rₙ, where Rₙ is optionally selected from hydrogen or C₁-C₅ alkyl. In a specific implementation, Rₙ is optionally selected from hydrogen, methyl, or ethyl.

In the compound of the present invention, a ring A has a structure shown in A1:

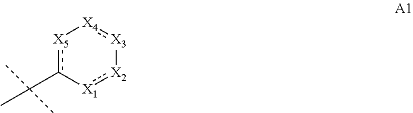

[symbol] optionally represents a single bond or a double bond, without violating the chemical bonding principle; and
X₁, X₂, X₃, X₄, and X₅ are each independently selected from a bond, C, N, O, C═O, or S(═O)ₘ.

That X₁, X₂, X₃, X₄, and X₅ are each selected from a bond indicates a direct linkage in the form of a bond, without violating the chemical bonding principle. For example, when X₂ is selected from a bond, X₁ is directly linked to X₃ by the bond, and then the ring A is a 5-membered ring.

In some specific implementations of the present invention, X₁, X₂, X₃, X₄, and X₅ are each independently selected from a bond, C, N, C═O, or S(═O)₂.

In some specific implementations of the present invention, X₁, X₂, X₃, X₄, and X₅ are all selected from C atoms.

In some specific implementations of the present invention, X₁, X₂, X₃, X₄, and X₅ optionally include one or two N atoms.

In some specific implementations of the present invention, X₁, X₂, X₃, X₄, and X₅ optionally include one C═O.

In some specific implementations of the present invention, X₁, X₂, X₃, X₄, and X₅ optionally include one S(═O)₂.

In some specific implementations of the present invention, X₁, X₂, X₃, X₄, and X₅ are optionally selected from a bond, C, S, and O.

In some specific implementations of the present invention, the ring A has the following structures:

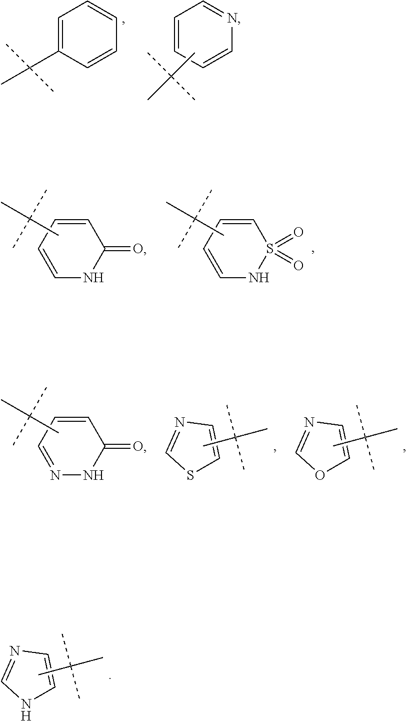

In the compound of the present invention, the ring A has a structure shown in A2:

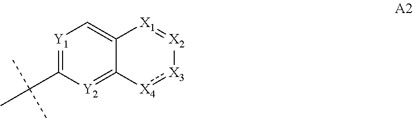

[symbol] optionally represents a single bond or a double bond, without violating the chemical bonding principle; and X₁, X₂, X₃, and X₄ are defined as in A1. That X₁, X₂, X₃, and X₄ are each selected from a “bond” indicates a direct linkage in the form of a bond. For example, when X₃ is selected from a bond, X₂ is directly linked to X₄ by the bond to form a 5-membered ring. Y₁ and Y₂ may be the same or different, and may be each optionally selected from a C atom or a N atom.

In some specific implementations of the present invention, X₁, X₂, X₃, and X₄ in A2 are each independently selected from a bond, C, N, or C═O.

In some specific implementations of the present invention, X₁, X₂, X₃, and X₄ in A2 are each independently selected from a bond, C, N, or O.

In some specific implementations of the present invention, X₁, X₂, X₃, and X₄ in A2 are each independently selected from a bond, C, N, or S(═O)ₘ.

In some specific implementations of the present invention, X₁, X₂, X₃, and X₄ in A2 are each independently selected from C, N, or C═O.

In some specific implementations of the present invention, the ring A has the following structures:

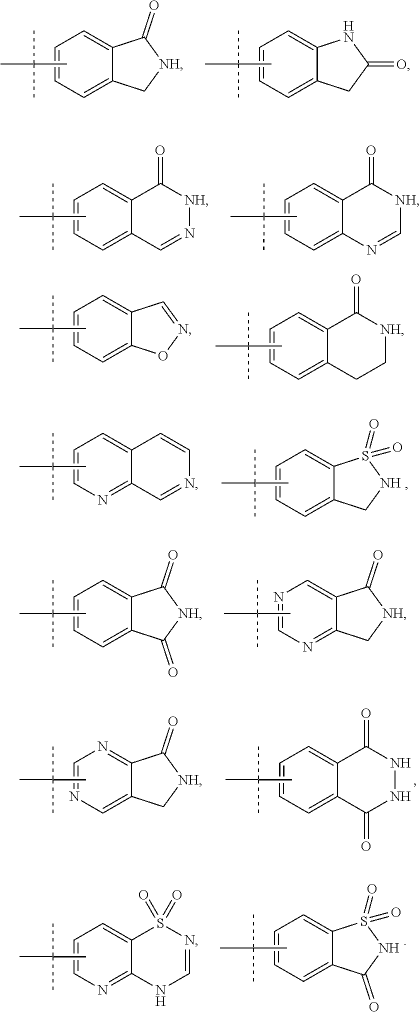

R_{c1} and R_{c2} are each independently selected from hydrogen, halogen, cyano, hydroxyl, amino, C₁-C₅ alkyl, C₁-C₅ alkoxy, —S(═O)ₘ—R_{c3}R_{c4}, —C(═O)—NR_{c3}R_{c4}, —C(═S)—NR_{c3}R_{c4}, —N(R_{c3}R_{c4})ₘ, —P(═O)ₘ—R_{c3}R_{c4}, —C(═N)—NR_{c3}R_{c4}, —S(═O)₂NR_{c3}R_{c4}, —CH₂NR_{c3}R_{c4}, or —S(═O)(═NR_{c3})R_{c4}; and the C₁-C₅ alkyl or C₁-C₅ alkoxy may be further substituted with one or more optionally selected from the following substituents: halogen, hydroxyl, cyano, amino, C₁-C₅ alkyl, and C₁-C₅ alkoxy.

R_{c1} and R_{c2} are substituted at any substitutable positions on the ring A, including at X₁, X₂, X₃, X₄, Y₁, and Y₂. In some specific implementations of the present invention, R_{c1} and R_{c2} are substituted at carbon atoms. In some specific implementations of the present invention, R_{c1} and R_{c2} are substituted at heteroatoms.

R_{c3} and R_{c4} are each independently selected from hydrogen, hydroxyl, amino, C₁-C₅ alkyl, C₁-C₅ alkoxy, or a 3- to 7-membered ring, or R_{c3} and R_{c4} are linked together to form a 3- to 7-membered ring; the 3- to 7-membered ring may be a saturated or unsaturated carbocyclic or heterocyclic ring; the heterocyclic ring optionally contains one or more N, O, or S(═O)ₘ heteroatoms; and the 3- to 7-membered ring may be further optionally substituted with one or more halogen, hydroxyl, cyano, C₁-C₅ alkyl, C₁-C₅ alkoxy, or amino.

When R_{c1} and R_{c2} are each selected from the substituent —S(═O)ₘ—R_{c3}R_{c4}, if m=2, any one of R_{c3} or R_{c4} is absent.

In some specific implementations of the present invention, the compound shown in formula I is further shown in general formula II:

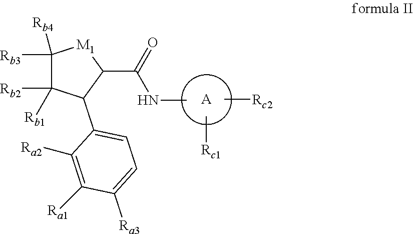

M₁, a ring A, Rₐ₁, Rₐ₂, Rₐ₃, R_{b1}, R_{b2}, R_{b3}, R_{b4}, R_{c1}, and R_{c2} are defined as above.

In some specific implementations of the present invention, R_{c1} and R_{c2} are each independently selected from hydrogen, hydroxyl, amino, fluorine, cyano, oxo, methyl, —C(═O)NH₂, —C(═O)NHCH₃, —C(═O)NHC₂H₅, —S(O)₂CH₃, —S(O)₂NH₂, —S(O)₂NHCH₃, —S(O)₂NHCD₃, —P(O)(CH₃)₂,

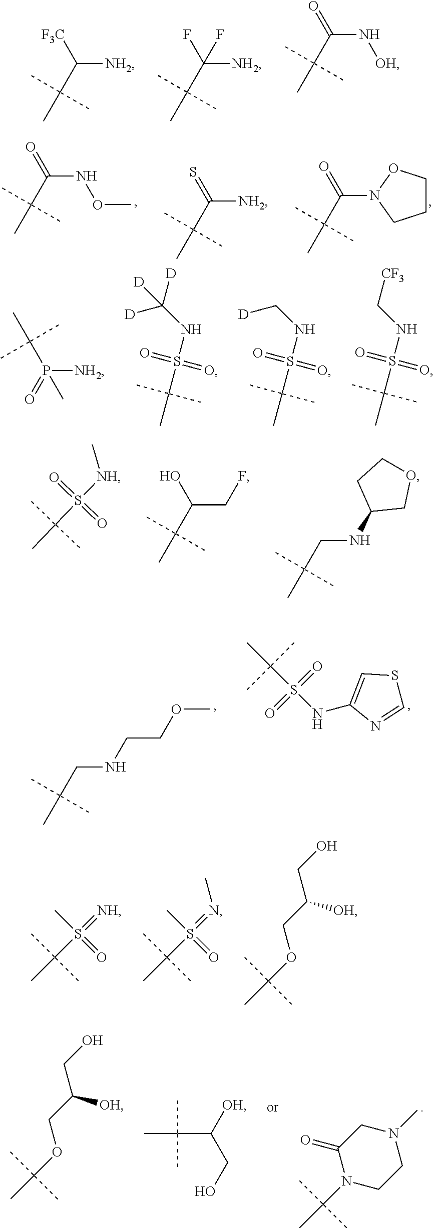

In some specific implementations of the present invention, R_{c1} and R_{c2} are each independently selected from hydrogen, fluorine, chlorine, amino, cyano, or —C(═O)NH₂.

In the compound of the present invention, m and n are each optionally 0, 1, or 2.

In a specific implementation of the present invention, a specific compound of the present invention has the following structures:

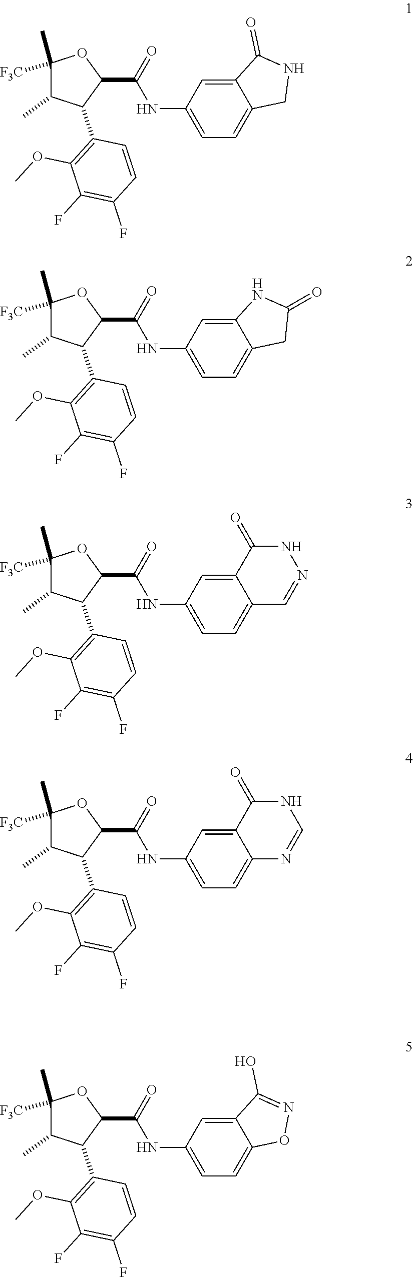

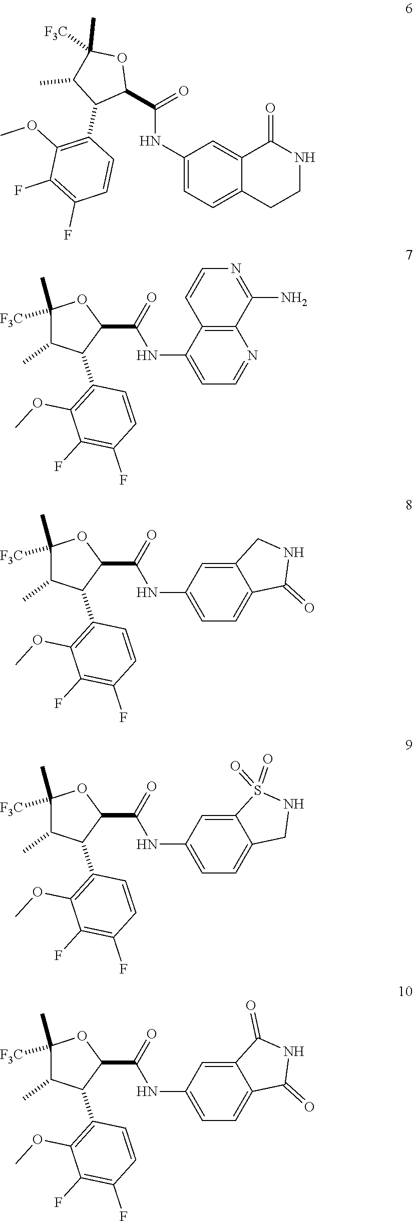

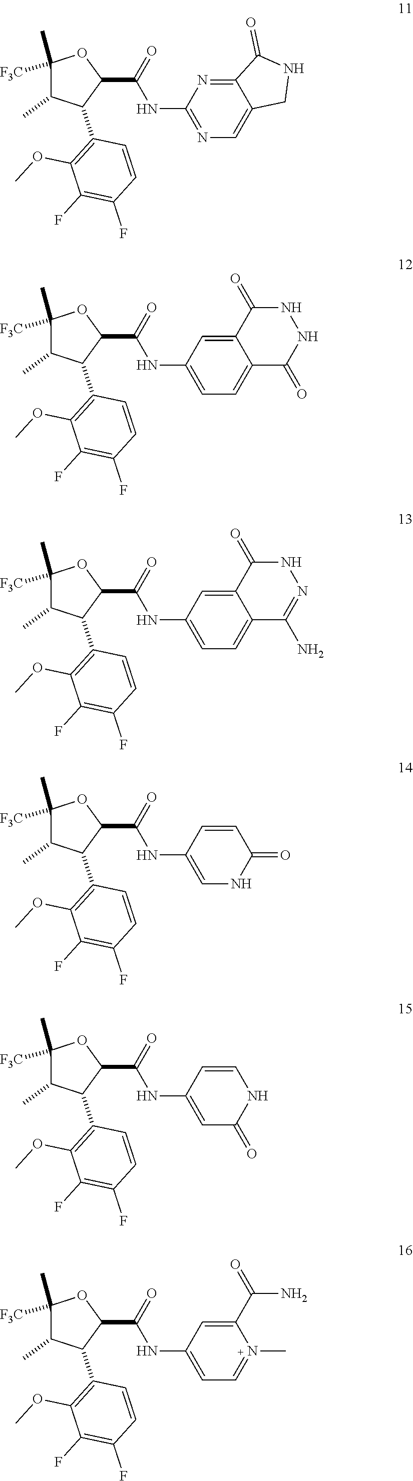

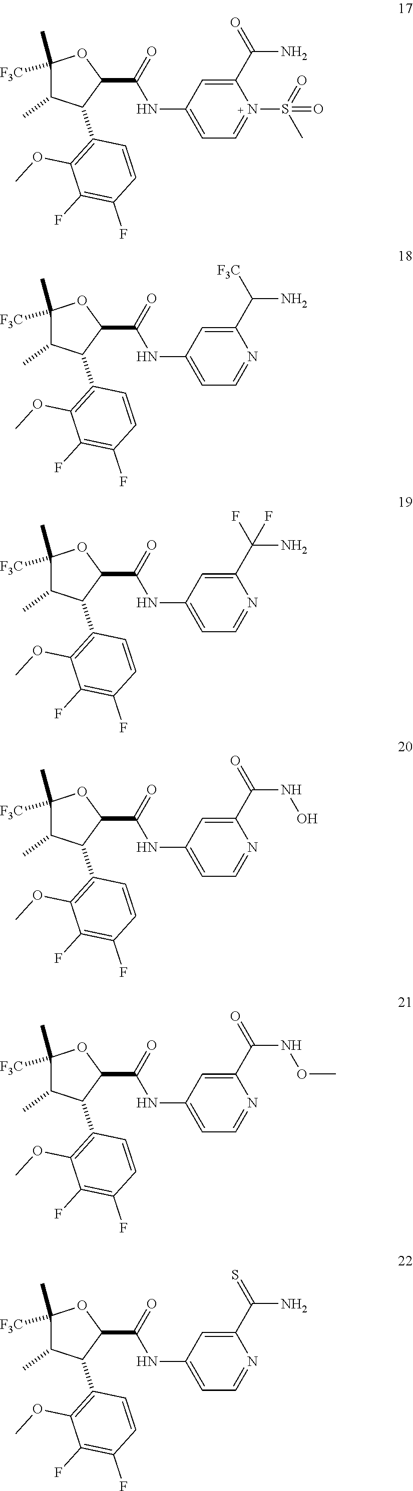

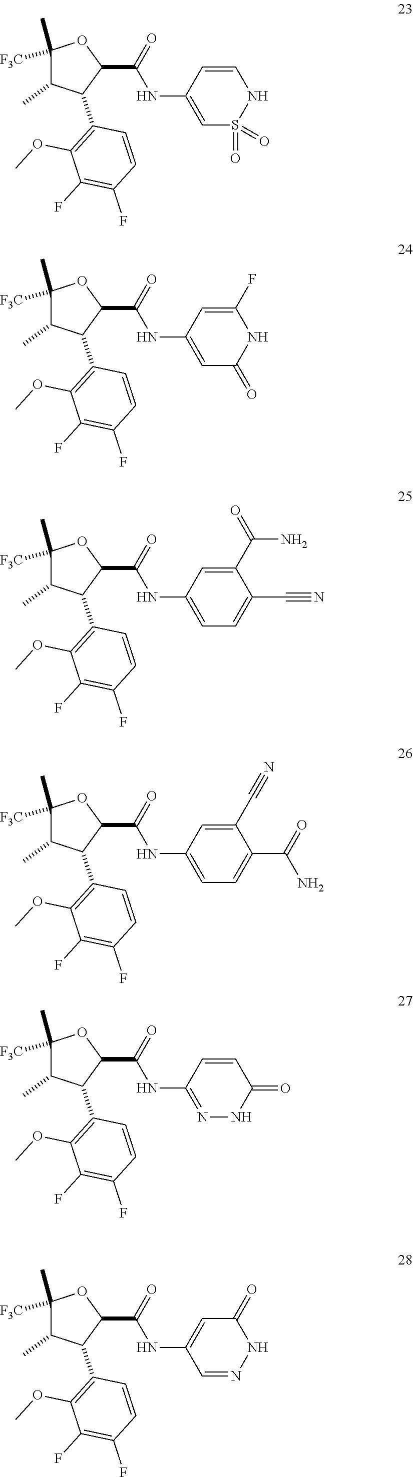

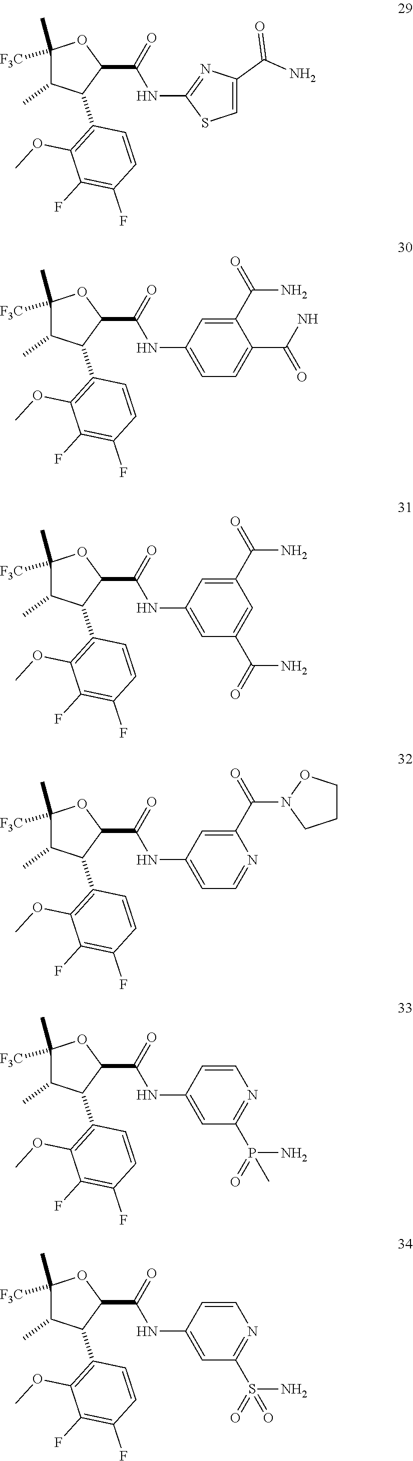

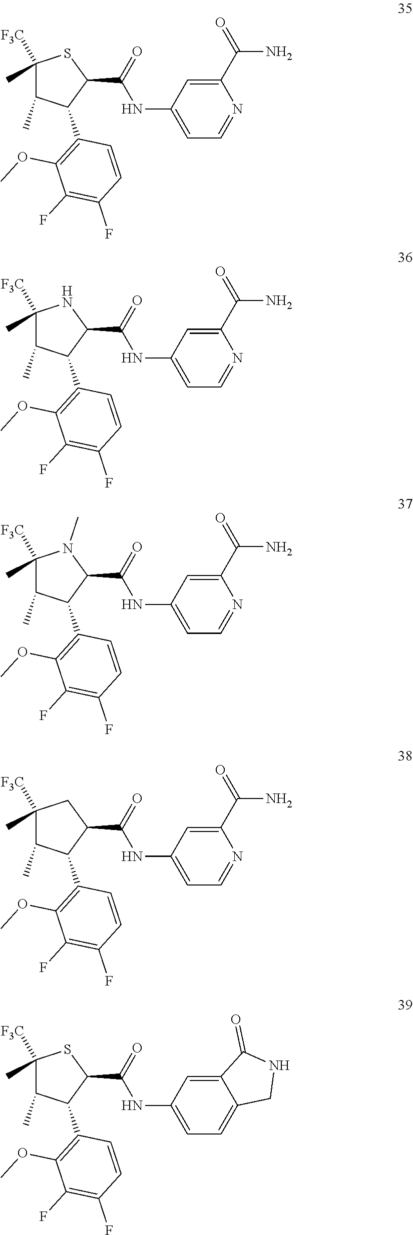

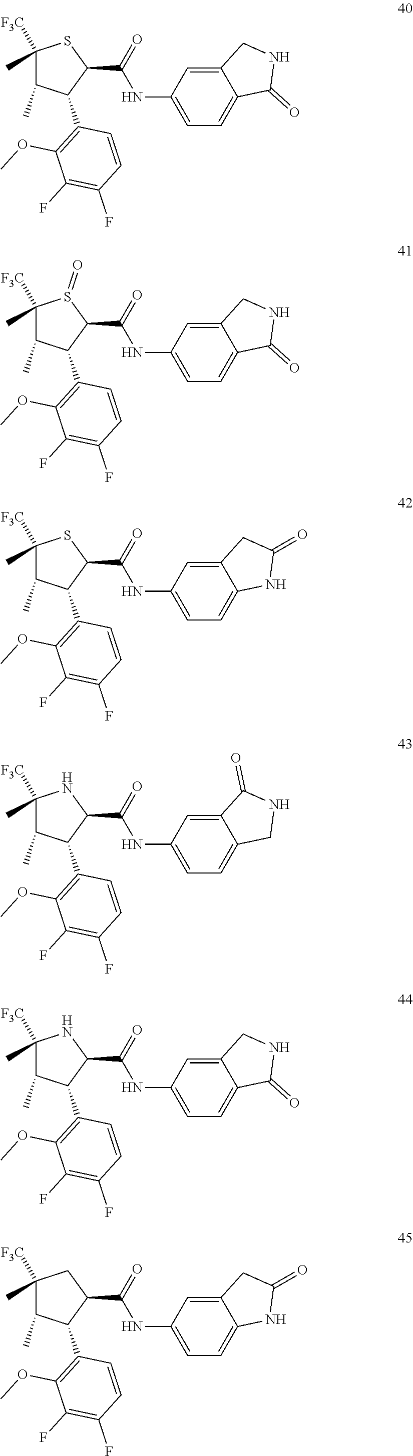 [SMILES: COc1c([C@H]2[C@H](C(=O)Nc3ccc4c(c3)CNC4=O)S[C@@](C)(C(F)(F)F)[C@H]2C)ccc(F)c1F ; COc1c([C@H]2[C@H](C(=O)Nc3ccc4c(c3)CNC4=O)S(=O)[C@@](C)(C(F)(F)F)[C@H]2C)ccc(F)c1F ; COc1c([C@H]2[C@H](C(=O)Nc3ccc4c(c3)CC(=O)N4)S[C@@](C)(C(F)(F)F)[C@H]2C)ccc(F)c1F ; COc1c([C@H]2[C@H](C(=O)Nc3ccc4c(c3)C(=O)NC4)N[C@@](C)(C(F)(F)F)[C@H]2C)ccc(F)c1F ; COc1c([C@H]2[C@H](C(=O)Nc3ccc4c(c3)CNC4=O)N[C@@](C)(C(F)(F)F)[C@H]2C)ccc(F)c1F ; COc1c([C@H]2[C@H](C(=O)Nc3ccc4c(c3)CC(=O)N4)C[C@@](C)(C(F)(F)F)[C@H]2C)ccc(F)c1F]

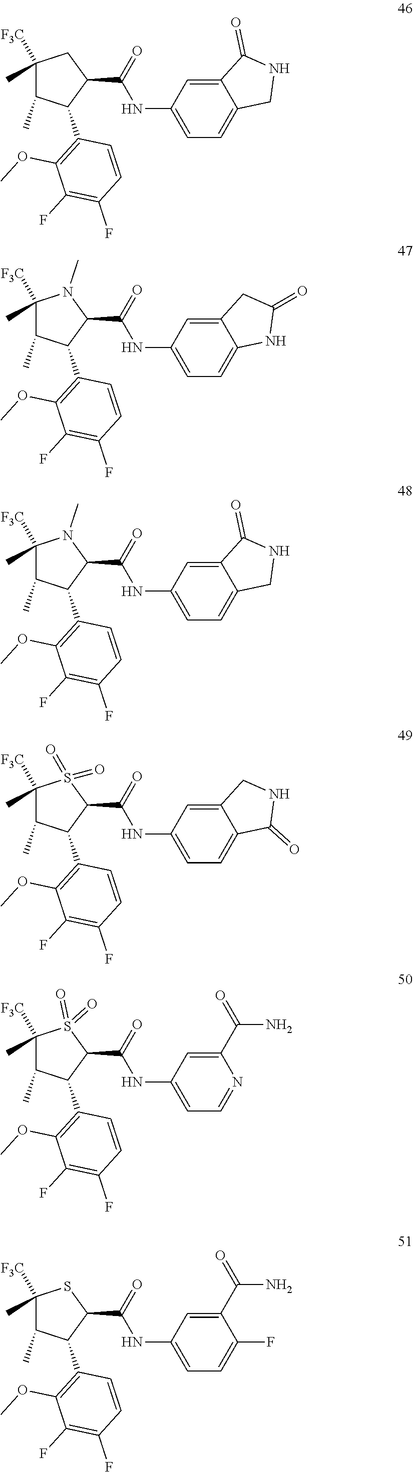

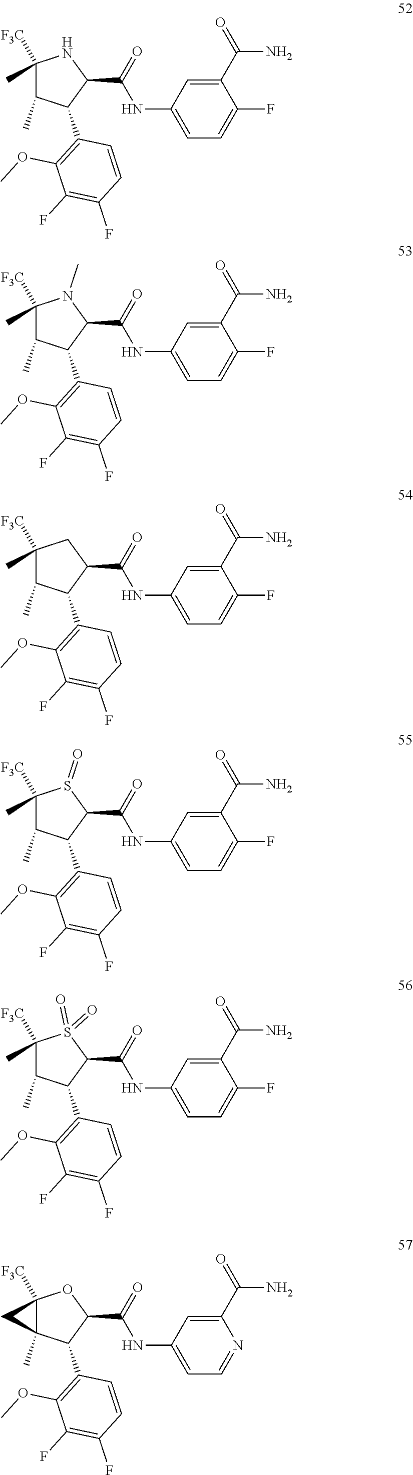

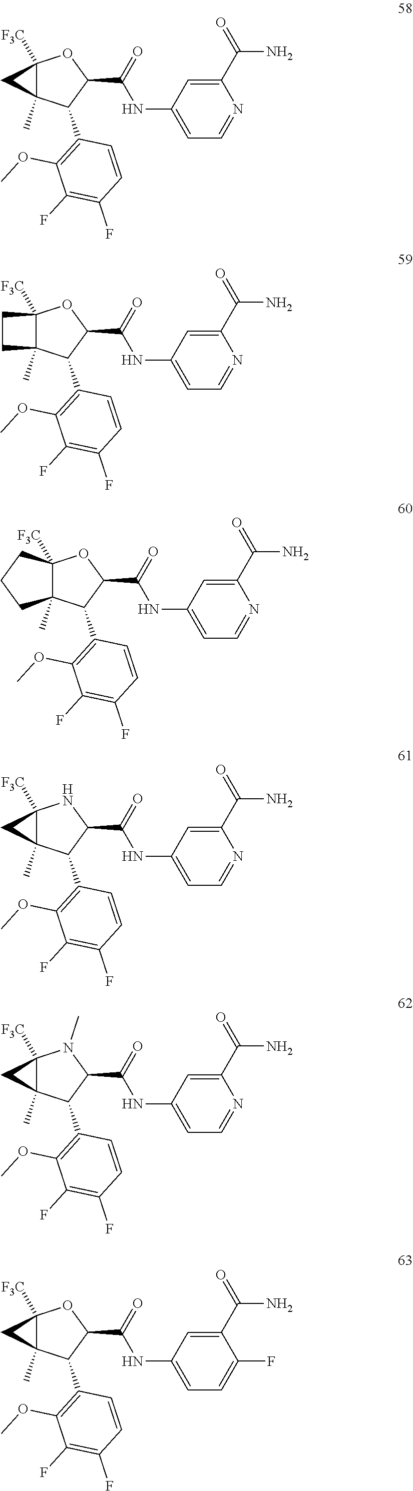

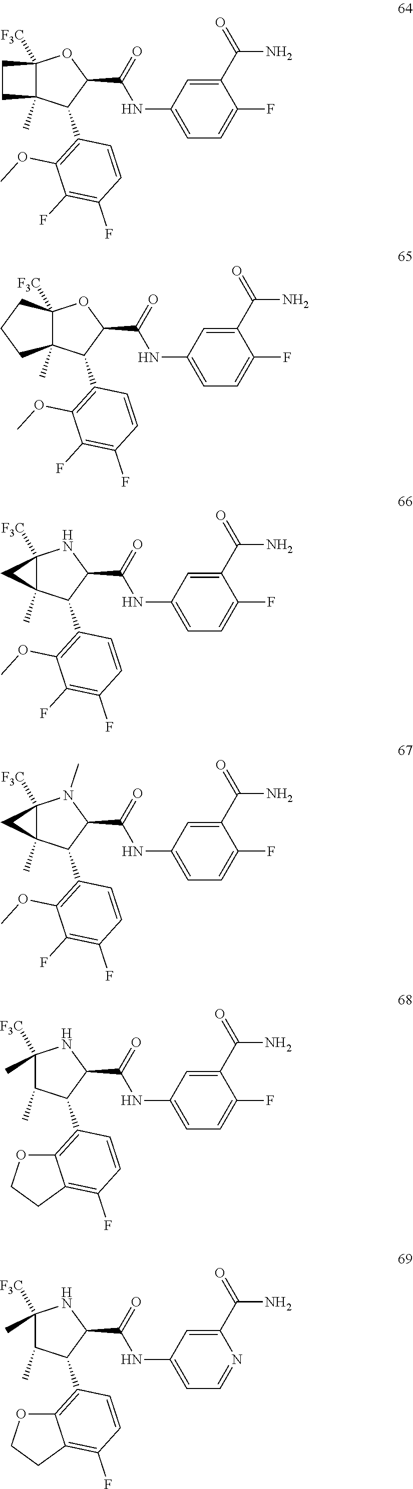 [SMILES: COc1c([C@H]2[C@H](C(=O)Nc3ccc(F)c(C(N)=O)c3)O[C@]3(C(F)(F)F)CC[C@]23C)ccc(F)c1F ; COc1c([C@H]2[C@H](C(=O)Nc3ccc(F)c(C(N)=O)c3)O[C@]3(C(F)(F)F)CCC[C@]23C)ccc(F)c1F ; COc1c([C@H]2[C@H](C(=O)Nc3ccc(F)c(C(N)=O)c3)N[C@]3(C(F)(F)F)C[C@]23C)ccc(F)c1F ; COc1c([C@H]2[C@H](C(=O)Nc3ccc(F)c(C(N)=O)c3)N(C)[C@]3(C(F)(F)F)C[C@]23C)ccc(F)c1F ; C[C@H]1[C@@H](c2ccc(F)c3c2OCC3)[C@H](C(=O)Nc2ccc(F)c(C(N)=O)c2)N[C@@]1(C)C(F)(F)F ; C[C@H]1[C@@H](c2ccc(F)c3c2OCC3)[C@H](C(=O)Nc2ccnc(C(N)=O)c2)N[C@@]1(C)C(F)(F)F]

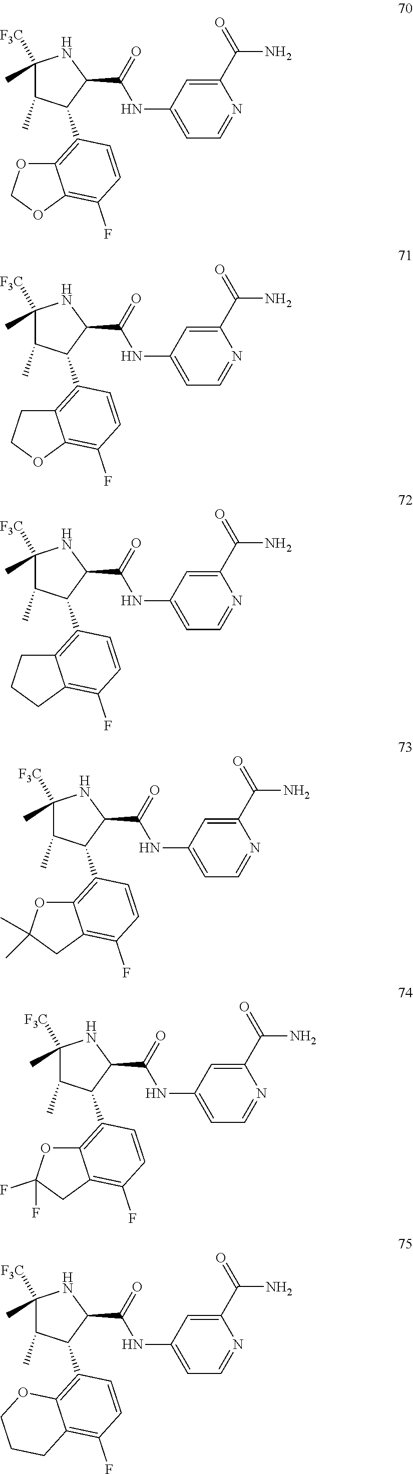 [SMILES: C[C@H]1[C@@H](c2ccc(F)c3c2OCO3)[C@H](C(=O)Nc2ccnc(C(N)=O)c2)N[C@@]1(C)C(F)(F)F ; C[C@H]1[C@@H](c2ccc(F)c3c2CCO3)[C@H](C(=O)Nc2ccnc(C(N)=O)c2)N[C@@]1(C)C(F)(F)F ; C[C@H]1[C@@H](c2ccc(F)c3c2CCC3)[C@H](C(=O)Nc2ccnc(C(N)=O)c2)N[C@@]1(C)C(F)(F)F ; C[C@H]1[C@@H](c2ccc(F)c3c2OC(C)(C)C3)[C@H](C(=O)Nc2ccnc(C(N)=O)c2)N[C@@]1(C)C(F)(F)F ; C[C@H]1[C@@H](c2ccc(F)c3c2OC(F)(F)C3)[C@H](C(=O)Nc2ccnc(C(N)=O)c2)N[C@@]1(C)C(F)(F)F ; C[C@H]1[C@@H](c2ccc(F)c3c2OCCC3)[C@H](C(=O)Nc2ccnc(C(N)=O)c2)N[C@@]1(C)C(F)(F)F]

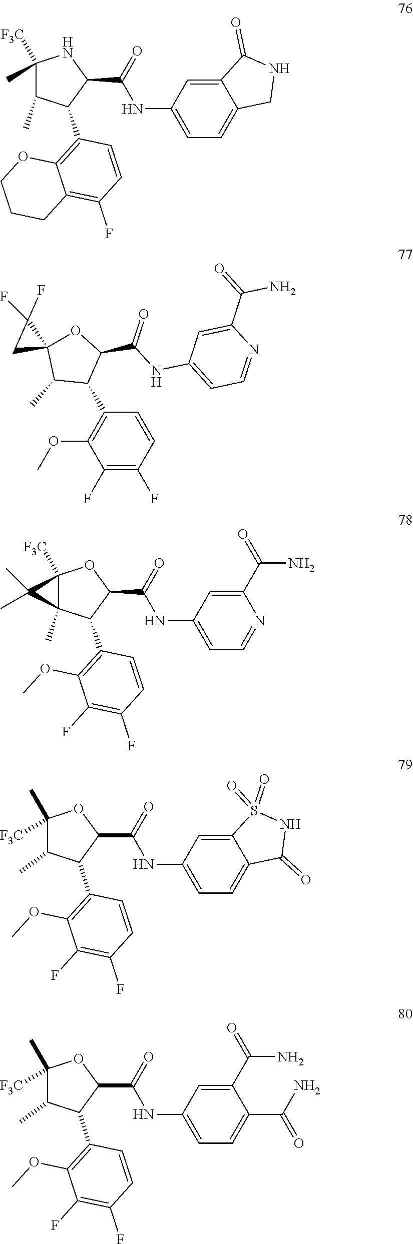

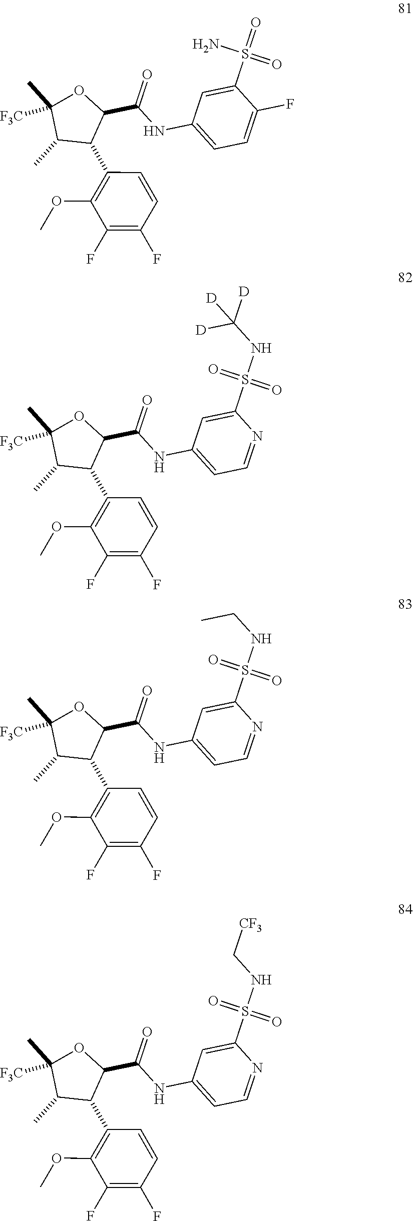

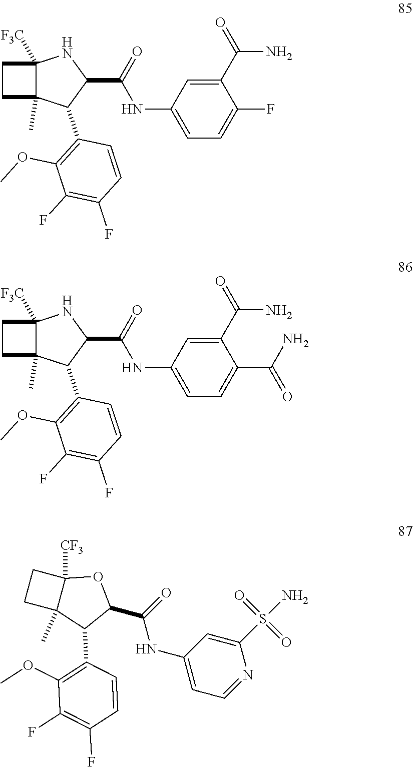

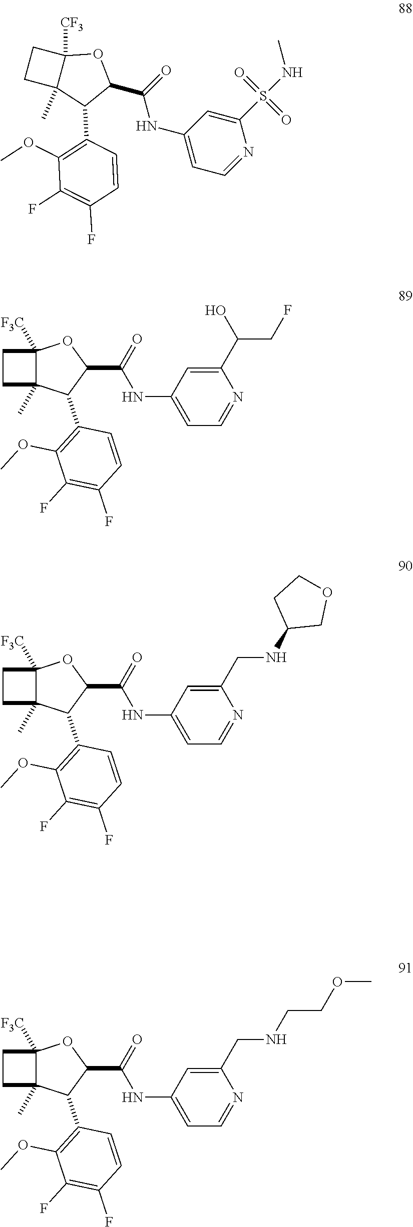

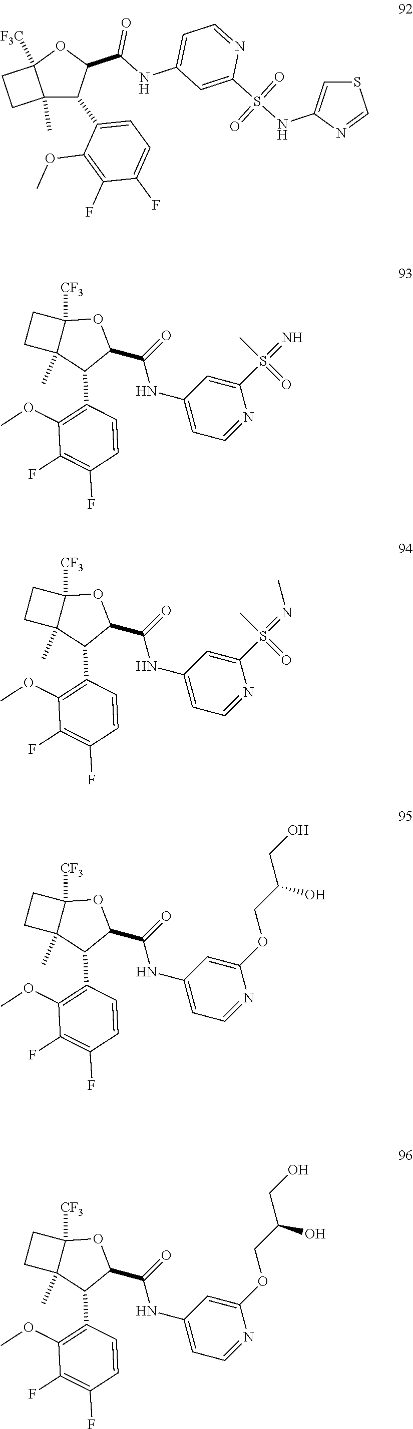

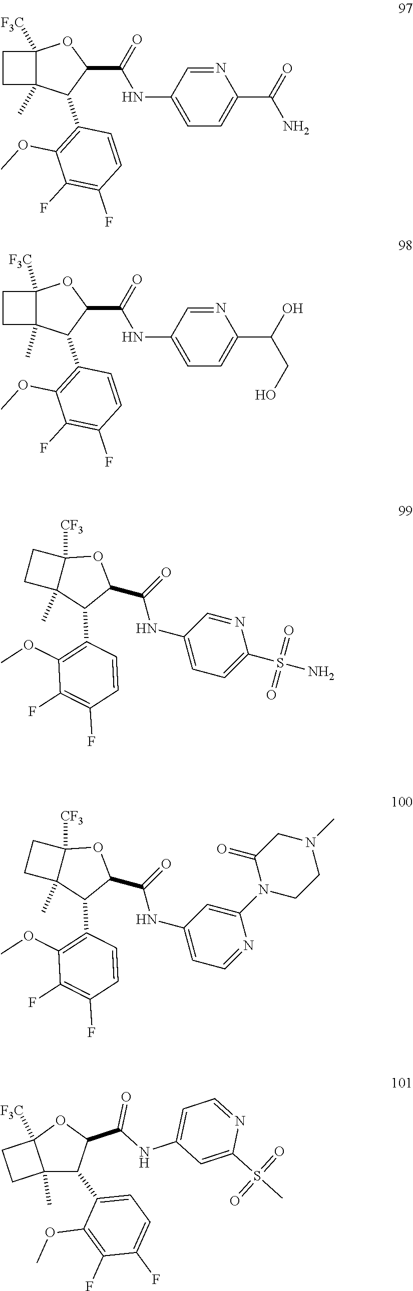

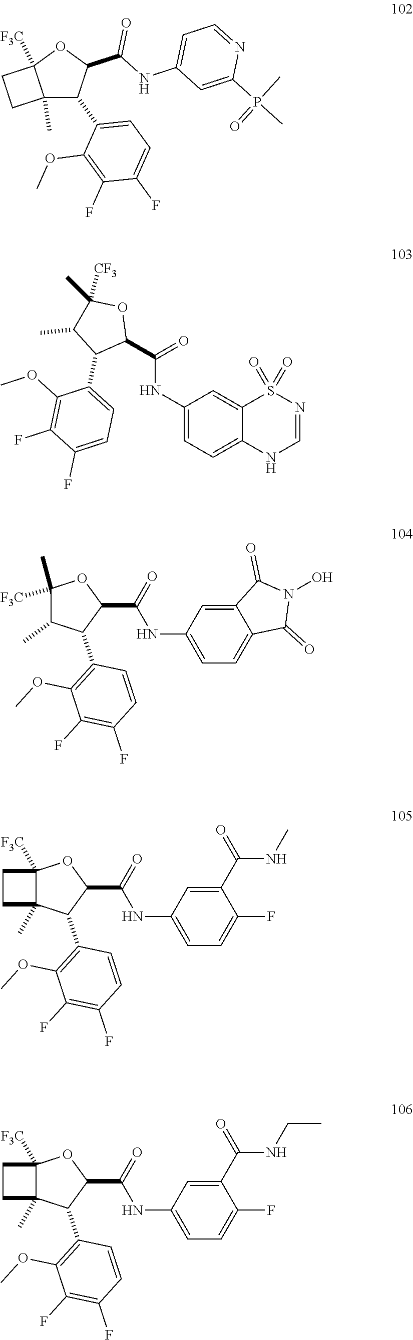

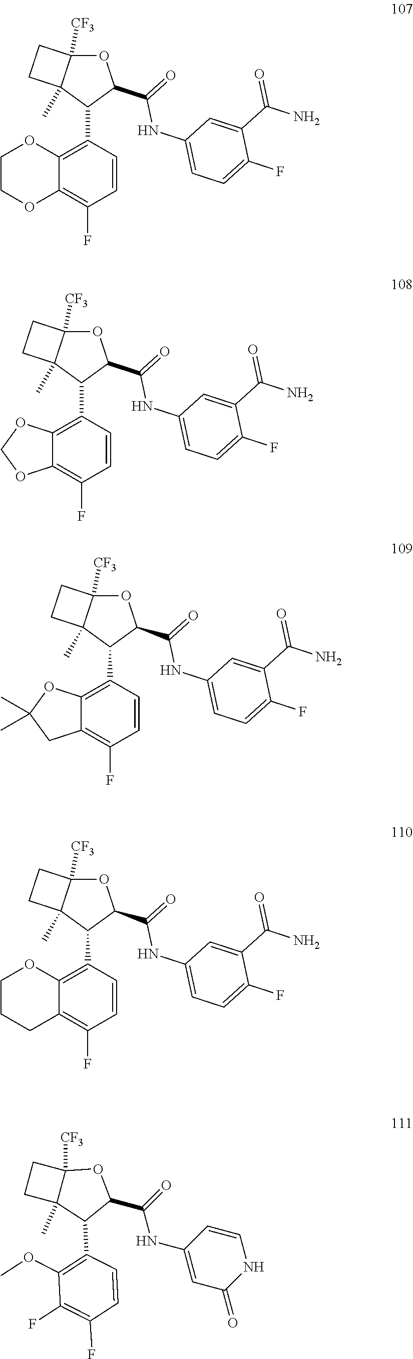

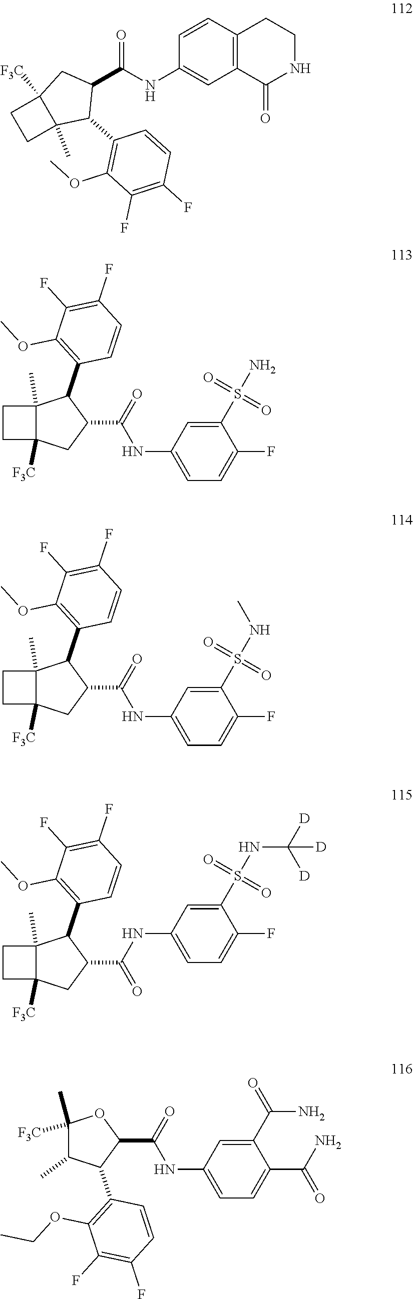

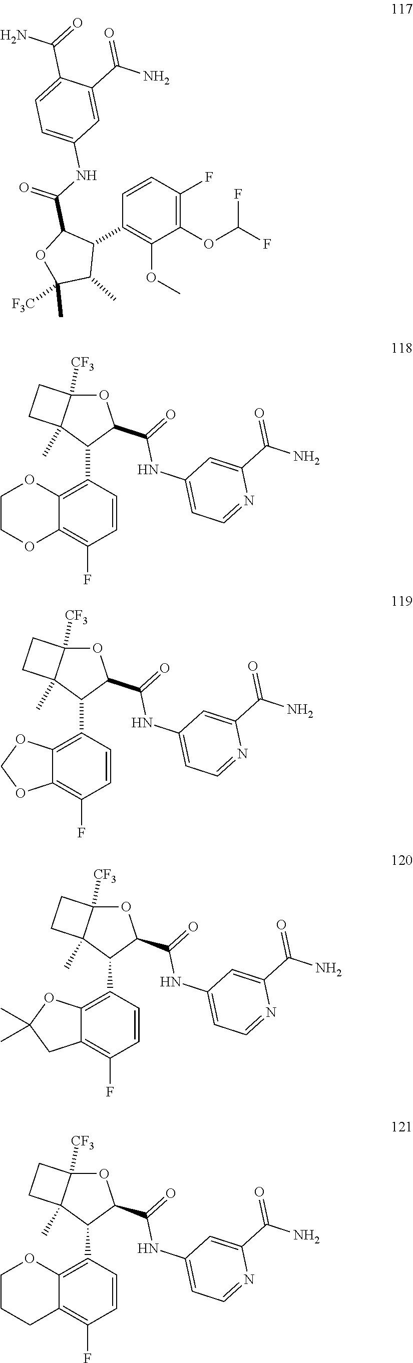

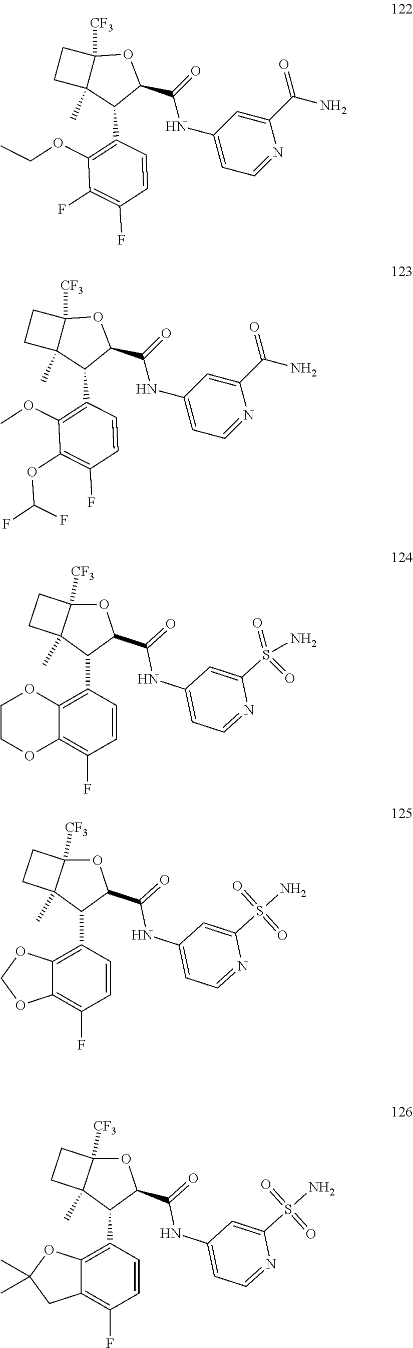

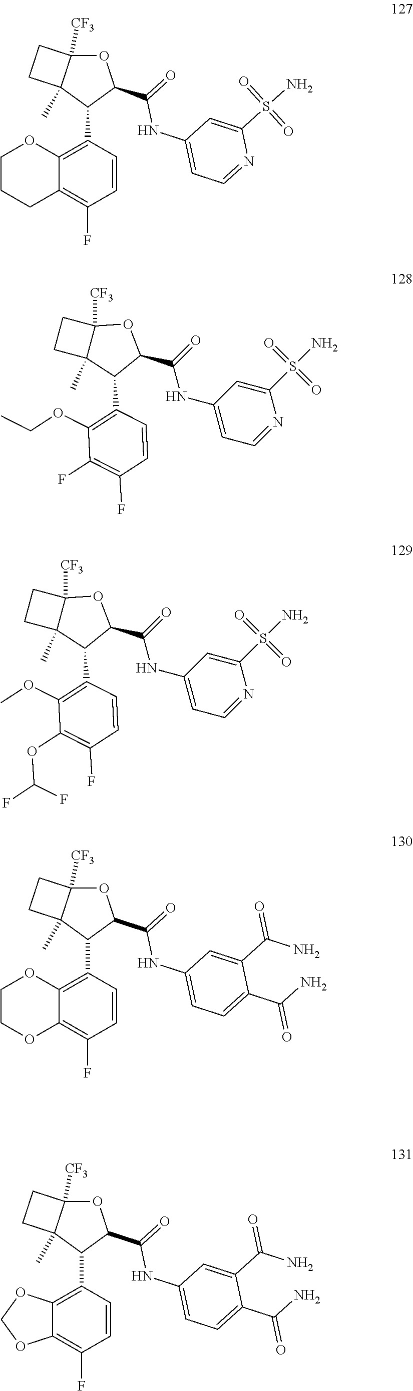

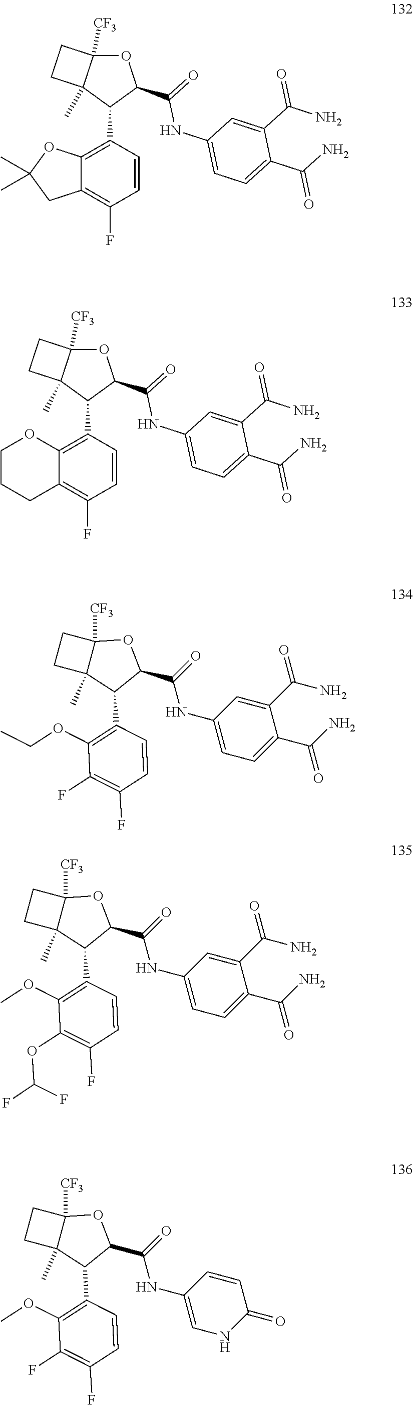

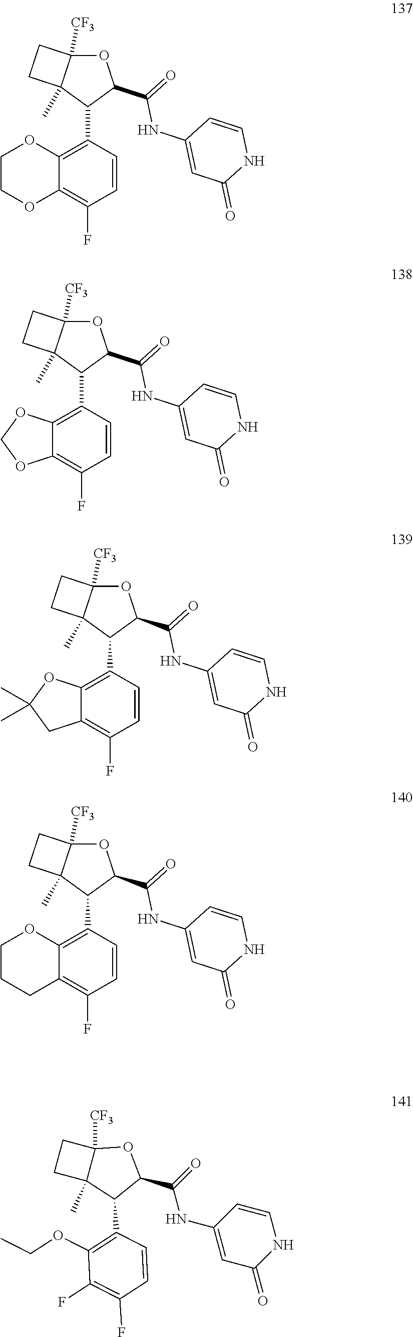

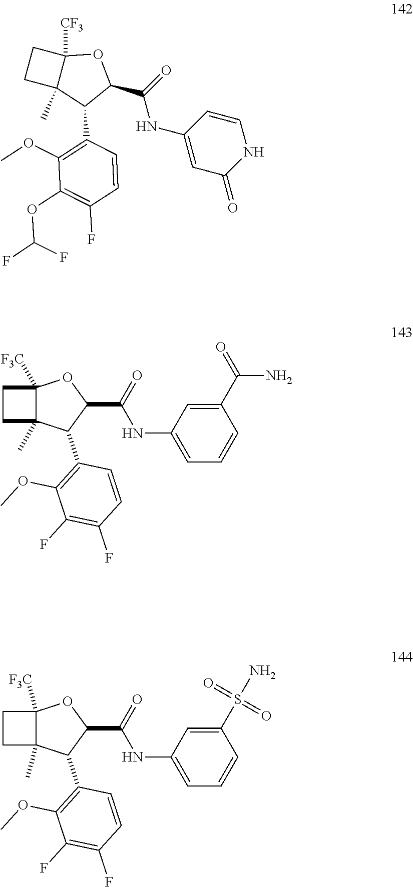

According to a second aspect of the present invention, provided is use of the compound shown in formula I or a pharmaceutically acceptable salt or a stereoisomer thereof in preparation of a drug for inhibition of voltage-gated sodium channels. The voltage-gated sodium channels are Naᵥ1.8.

According to a third aspect of the present invention, provided is use of the compound shown in formula I or a pharmaceutically acceptable salt or a stereoisomer thereof in preparation of a drug for the treatment, prevention, or relief of a disease related to voltage-gated sodium channels, where the related disease includes, but is not limited to: pain, multiple sclerosis, Charcot-Marie-Tooth disease, incontinence, pathologic coughs, or arrhythmia.

The pain includes: acute pain, chronic pain, intestinal pain, neuropathic pain, musculoskeletal pain, inflammatory pain, cancer pain, idiopathic pain, postoperative pain, and visceral pain. Therefore, the present invention further provides use of the compound shown in formula I or a pharmaceutically acceptable salt thereof in preparation of a drug for treatment, prevention, or relief of pain.

According to a fourth aspect of the present invention, provided is a pharmaceutical composition, where the pharmaceutical composition contains the compound shown in formula I or a pharmaceutically acceptable salt or a stereoisomer thereof, and a pharmaceutically acceptable excipient. The pharmaceutically acceptable excipient includes a filler, a disintegrant, a surfactant, and a solubilizer.

In the present invention, the pharmaceutical composition containing the compound shown in formula I or the pharmaceutically acceptable salt thereof may be administered in various known ways, such as orally, topically, rectally, parenterally, by inhalation, or by implantation. The pharmaceutical composition may be prepared in the form of tablets, capsules, powder in packets, sugar-coated pills, dispersions, granules, lozenges, powder for injection, liquid formulations, suppositories, or other formulations.

According to a fifth aspect of the present invention, provided is use of the pharmaceutical composition containing the compound shown in formula I or a pharmaceutically acceptable salt or a stereoisomer thereof in preparation of a drug for treatment, prevention, or relief of a disease related to voltage-gated sodium channels, where the disease related to voltage-gated sodium channels includes, but is not limited to: pain, multiple sclerosis, Charcot-Marie-Tooth disease, incontinence, pathologic coughs, or arrhythmia.

The pain includes: acute pain, chronic pain, intestinal pain, neuropathic pain, musculoskeletal pain, inflammatory pain, cancer pain, idiopathic pain, postoperative pain, and visceral pain. Therefore, the present invention further provides use of the pharmaceutical composition containing the compound shown in formula I or a pharmaceutically acceptable salt thereof in preparation of a drug for treatment, prevention, or relief of pain.

### DETAILED DESCRIPTION

### Term Description

“Halogen” or “halogen atom” includes fluorine, chlorine, bromine, and iodine. In the present invention, the halogen is preferably fluorine or chlorine.

“C₁-C₅ alkyl” is straight- or branched-chain alkyl containing 1-5 carbon atoms, such as methyl, ethyl, propyl, isopropyl, n-butyl, isobutyl, tert-butyl, or n-pentyl. Preferably, C₁-C₅ alkyl is C₁-C₃ alkyl, including methyl, ethyl, propyl, or isopropyl.

“C₁-C₅ alkoxy” is straight- or branched-chain alkoxy containing 1-5 carbon atoms, such as methoxy, ethoxy, propoxy, isopropoxy, or tert-butoxy. Preferably, C₁-C₅ alkoxy is C₁-C₃ alkoxy, including methoxy, ethoxy, propoxy, or isopropoxy. “C₃-C₆ cycloalkyl” is cycloalkyl containing 3-6 carbon atoms, including cyclopropyl, cyclobutyl, cyclopentyl, or cyclohexyl. On the cycloalkyl, there may be further substitutions with the following substituents: one or more halogen, hydroxyl, cyano, C₁-C₅ alkyl, C₁-C₅ alkoxy, and amino.

“C₃-C₆ cycloalkoxy” is cycloalkoxy containing 3-6 carbon atoms, including cyclopropoxy, cyclobutoxy, cyclopentyloxy, or cyclohexyloxy. On the cycloalkoxy, there may be further substitutions with the following substituents: one or more halogen, hydroxyl, cyano, C₁-C₅ alkyl, C₁-C₅ alkoxy, and amino.

“3- to 7-membered ring” is a saturated or unsaturated ring containing 3-7 carbon atoms, such as a 3-membered ring, a 4-membered ring, a 5-membered ring, a 6-membered ring, or a 7-membered ring. In the present invention, a carbon atom on the ring may be optionally substituted with one or more C(═O), N, O, or S(═O)ₘ to form a 3- to 7-membered heterocyclic ring, and an atom on the 3- to 7-membered ring may be further substituted optionally with one or more halogen, hydroxyl, cyano, C₁-C₅ alkyl, C₁-C₅ alkoxy, or amino, where m is 0, 1, or 2.

“5- to 7-membered ring” is a saturated or unsaturated ring containing 5-7 carbon atoms, such as a 5-membered ring, a 6-membered ring, or a 7-membered ring. In the present invention, a carbon atom on the ring may be optionally substituted with one or more N, O, or S(═O)ₘ to form a 5- to 7-membered heterocyclic ring, and an atom on the 5- to 7-membered ring may be further substituted optionally with one or more halogen, hydroxyl, cyano, C₁-C₅ alkyl, C₁-C₅ alkoxy, or amino, where m is 0, 1, or 2.

“More” may indicate two, three, four, or more. In the present invention, a substitution with more halogen may form, for example, trifluoromethyl or difluoromethyl.

In the present invention, the alkyl, alkoxy, cycloalkyl, cycloalkoxy, 5- to 7-membered ring, or 3- to 7-membered ring may be optionally substituted with one or more deuterium, halogen, hydroxyl, cyano, oxo, C₁-C₅ alkyl, C₁-C₅ alkoxy, or amino.

As used herein, in any chemical structure or formula, a bold or hashed straight bond (respectively [symbol] or [symbol] linked to a stereoisomeric center of a compound, for example, in

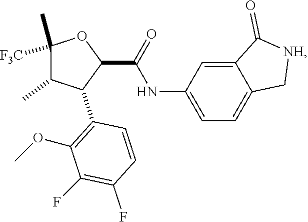

indicates the relative stereochemistry of the stereoisomeric center relative to another stereoisomeric center to which the bold or hashed straight bond is linked.

As used herein, in any chemical structure or formula, a bold or hashed wedge bond (respectively [symbol] or [symbol] linked to a stereoisomeric center of a compound, for example, in

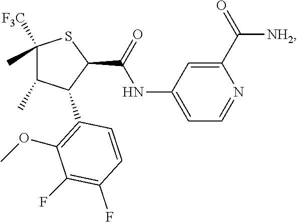 [SMILES: COc1c([C@H]2[C@H](C(=O)Nc3ccnc(C(N)=O)c3)S[C@@](C)(C(F)(F)F)[C@H]2C)ccc(F)c1F]

indicates the absolute stereochemistry of the stereoisomeric center and the relative stereochemistry of the stereoisomeric center relative to another stereoisomeric center to which the bold or hashed wedge bond is linked.

As used herein, the prefix “rel-” indicates a single enantiomer with an unknown absolute configuration when used in combination with a chiral compound. In a compound with the prefix “rel-”, the (R)- and (S)- notations in the chemical name reflect the relative stereochemistry of the compound, but not necessarily the absolute stereochemistry of the compound.

“Pharmaceutically acceptable salt” includes, but is not limited to: an acid addition salt formed from the compound shown in formula I and an inorganic acid, such as hydrochloride, hydrobromide, phosphate, sulfate, or nitrate; and an acid addition salt formed from the compound shown in formula I and an organic acid, such as formate, acetate, malate, maleate, fumarate, tartrate, succinate, citrate, lactate, methanesulfonate, p-toluenesulfonate, 2-hydroxyethylsulfonate, or benzoate.

The compound in the present invention further includes a tautomer. For example, tautomers of enol and keto structures belong to the same compound.

In the present invention, specific compounds are further prepared by the following methods. Unless otherwise specified, compounds, reagents, and the like used in examples of the present invention are purchased from qualified suppliers or synthesized according to methods disclosed in the related art. Some intermediate compounds may be synthesized according to the methods described in WO2021113627A1. For example, intermediates 1a and 16a are prepared according to the methods in the related art.

### Example 1

### rel-(2R,3S,4S,5R)-3-(3,4-difluoro-2-methoxyphenyl)-4,5-dimethyl-N-(3-oxoisoindolin-5-yl)-5-(trifluoromethyl)tetrahydrofuran-2-carboxamide

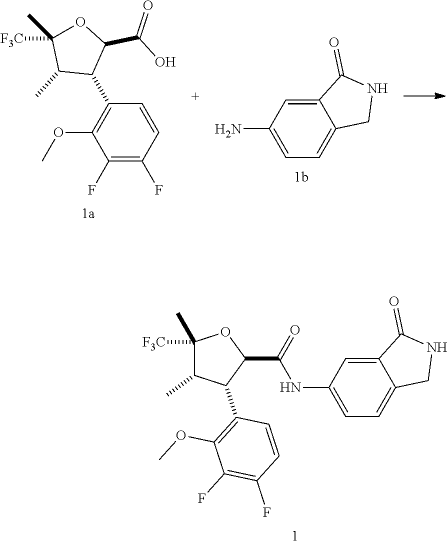

rel-(2R,3S,4S,5R)-3-(3,4-difluoro-2-methoxyphenyl)-4,5-dimethyl-5-(trifluoromethyl)tetrahydrofuran-2-carboxylic acid (1a, 100 mg, 0.28 mmol) was added in a reaction flask, dichloromethane (25 mL) and 1 drop of N,N-dimethylformamide were added, and oxalyl chloride (72 mg, 0.56 mmol) was added dropwise under nitrogen protection and in an ice-water bath. After the dropwise addition, the ice-water bath was removed, and the reaction system was warmed to room temperature, stirred for 1 h, and concentrated to remove the reaction solvent. The residue was dissolved in dichloromethane (25 mL), and a 6-aminoisoindolin-1-one (1b, 50 mg, 0.34 mmol) in dichloromethane (10 mL) solution was added dropwise at −10° C. After the dropwise addition, the reaction system was warmed to room temperature and stirred for 1 h. The reaction solution was quenched with water (25 mL). After separation in layers, the aqueous phase was subjected to extraction with DCM twice. The organic phases were combined and washed with water and saturated saline once each. The organic phase was dried over anhydrous sodium sulfate, then concentrated, and then separated by column chromatography, to obtain a target product (102 mg, 75% yield). ESI-MS m/z calc. 484.4, found 485.1 (M+1)⁺. ¹H NMR (400 MHz, DMSO-d6) δ 10.59 (s, 1H), 8.54 (s, 1H), 8.01 (d, J=1.8 Hz, 1H), 7.76 (dd, J=8.2, 1.9 Hz, 1H), 7.50 (d, J=8.2 Hz, 1H), 7.23-7.12 (m, 1H), 5.14 (d, J=10.4 Hz, 1H), 4.30 (s, 2H), 4.26 (dd, J=10.4, 7.6 Hz, 1H), 3.95 (d, J=1.9 Hz, 3H), 2.76 (dt, J=14.7, 7.4 Hz, 1H), 1.61 (s, 3H), 0.73 (d, J=6.3 Hz, 3H).

### Example 2: rel-(2R,3S,4S,5R)-3-(3,4-difluoro-2-methoxyphenyl)-4,5-dimethyl-N-(2-oxoisoindolin-6-yl)-5-(trifluoromethyl)tetrahydrofuran-2-carboxamide

Example 2 was obtained by a method similar to that in Example 1 with 6-aminoisoindolin-2-one that replaced 6-aminoisoindolin-1-one in Example 1. ESI-MS m/z calc. 484.4, found 485.1 (M+1)⁺. ¹H NMR (400 MHz, DMSO-d6) δ 10.37 (s, 1H), 10.32 (s, 1H), 7.30 (s, 1H), 7.20-7.14 (m, 2H), 7.11-7.05 (m, 2H), 5.08 (d, J=10.4 Hz, 1H), 4.27-4.19 (m, 1H), 3.95 (d, J=2.0 Hz, 3H), 3.39 (s, 2H), 2.78-2.70 (m, 1H), 1.58 (s, 3H), 0.72 (d, J=6.1 Hz, 3H).

### Example 3

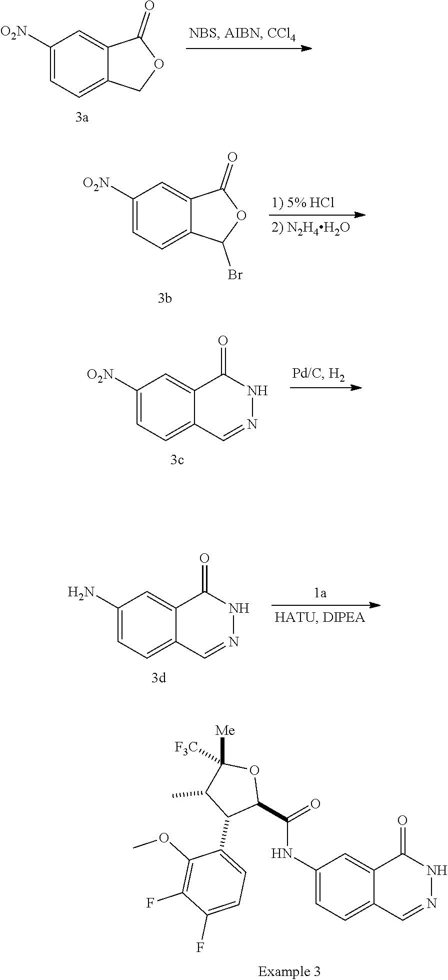

### Step 1: 3-bromo-6-nitrobenzofuran-1(3H)-one (3b)

3a (3.0 g) and CCl₄ (30 mL) were added in a 100 mL reaction flask, and NBS (3.28 g) and AIBN (275 mg) were added sequentially while stirring, then heated to 78° C. and stirred for 12 h. The reaction was monitored by TLC until the raw material was fully converted. The reaction solution was cooled down to room temperature and filtered with diatomaceous earth. The filtrate was concentrated and separated by column chromatography (EA/PE, 0%-30%), to obtain 3.3 g of yellow solid with a yield of 77%. LCMS: m/z (254 nm): 257.9 [M+H]⁺

### Step 2: 7-nitrophthalazin-1(2H)-one (3c)

3b (1.0 g) and 5% HCl (5 mL/mmol) were added in a reaction flask and stirred at 80° C. for 3 h. The reaction was monitored by TLC until the raw material was fully converted. The reaction solution was cooled down to room temperature. N₂H₄H₂O (274 mg) was added and stirred at room temperature for 3 h. The reaction was monitored by TLC until the raw material was fully converted. The reaction solution was filtered and washed with water. The solid residue was separated by column chromatography to obtain 237 mg of white solid with a yield of 32%. LCMS: m/z (254 nm): 192.0 [M+H]+

### Step 3: 7-aminophthalazin-1(2H)-one (3d)

3c (200 mg), MeOH (10 mL), and THF (10 mL) were added in a reaction flask and stirred uniformly. Then, palladium on carbon (20 mg) was added, with hydrogen displacement for three times, and stirred at room temperature for 4 h. The reaction was monitored by TLC until the raw material was fully converted. The reaction solution was filtered with diatomaceous earth. The filtrate was concentrated to obtain 120 mg of white solid with a yield of 71%. LCMS: m/z (254 nm): 162.0 [M+H]⁺

### Step 4: rel-(2R,3S,4S,5R)-3-(3,4-difluoro-2-methoxyphenyl)-4,5-dimethyl-N-4-oxa-3,4-dihydrophthalazin-6-yl)-5-(trifluoromethyl)tetrahydrofuran-2-carboxamide (Example 3)

1a (30 mg), DMF (1 mL), HATU (49 mg), and DIPEA (22 mg) were added in a 25 mL reaction flask and stirred for 5 min. Then, 3d (17 mg) was added and stirred at room temperature for 12 h. The reaction was monitored by LCMS until the raw material was fully converted. The reaction solution with water (10 mL) added was subjected to extraction with ethyl acetate (10 mL×3). The organic phases were combined, dried over anhydrous sodium sulfate, filtered, concentrated, and separated by silica gel plate chromatography (EA/PE, 50%), to obtain 11.2 mg of white solid with a yield of 26.6%. LCMS: m/z (254 nm): 498.1 [M+H]⁺. ¹H NMR (400 MHz, DMSO-d6) δ 12.55 (s, 1H), 10.71 (s, 1H), 8.56 (d, J=2.0 Hz, 1H), 8.27 (s, 1H), 8.08 (dd, J=8.6, 2.2 Hz, 1H), 7.89 (d, J=8.6 Hz, 1H), 7.20-7.12 (m, 2H), 5.13 (d, J=10.3 Hz, 1H), 4.29 (dd, J=10.1, 7.8 Hz, 1H), 3.95 (d, J=2.0 Hz, 3H), 2.84-2.72 (m, 1H), 1.62 (s, 3H), 0.74 (d, J=6.1 Hz, 3H).

### Example 4: rel-(2R,3S,4S,5R)-3-(3,4-difluoro-2-methoxyphenyl)-4,5-dimethyl-N-(4-oxo-3H-quinazolin-6-yl)-5-(trifluoromethyl)tetrahydrofuran-2-carboxamide (4)

Example 4 was obtained by a method similar to that in Example 1 with 6-amino-3,4-dihydroquinazolin-4-one that replaced 1b in Example 1. ESI-MS m/z calc. 484.4, found 485.1 (M+1)⁺. ¹H NMR (400 MHz, DMSO-d₆) δ 12.25-12.16 (m, 1H), 10.60 (s, 1H), 8.51 (d, J=2.5 Hz, 1H), 8.11-7.94 (m, 2H), 7.67 (d, J=8.8 Hz, 1H), 7.28-7.15 (m, 2H), 5.14 (d, J=10.3 Hz, 1H), 4.32 (dd, J=10.3, 7.6 Hz, 1H), 3.99 (d, J=2.1 Hz, 3H), 2.81 (t, J=7.5 Hz, 1H), 1.66 (s, 3H), 0.78 (dd, J=7.4, 2.5 Hz, 3H).

### Example 5

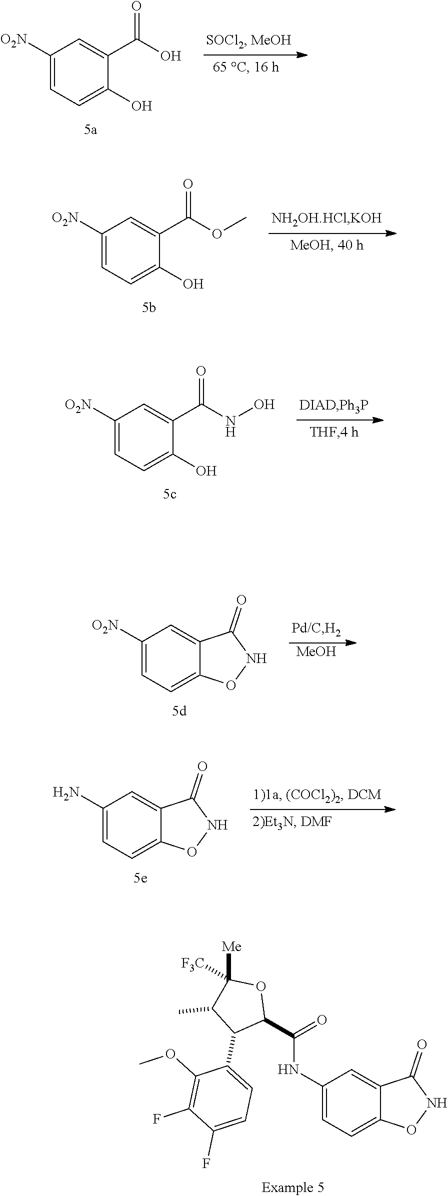

### Step 1: methyl 2-hydroxy-5-nitrobenzoate (5b)

Methanol (50 mL) and 5a (5.0 g) were added in a three-necked flask, and sulfoxide chloride (10 mL) was slowly added dropwise to react at 65° C. for 16 h, and then concentrated until dried. Water and ethyl acetate were added to carry out extraction. The ethyl acetate layer was taken and concentrated to obtain 6.0 g of white solid with a yield of 111%. ESI-MS m/z calc. 197.2, found 198.1 (M+1)⁺.

### Step 2: (2-hydroxy-5-nitrophenyl)methane hydroxamic acid (5c)

Methanol (500 mL) and potassium hydroxide (21 g) were added in a reaction flask. Hydroxylamine hydrochloride (17.5 g) was added and stirred in an ice bath for 1.5 h, and then filtered to obtain a filtrate. 5b (5 g) was added to the filtrate to react for 16 h, until the completion of the reaction was detected by LCMS. The reaction system was concentrated to remove methanol, adjusted with hydrochloric acid to pH 2, and subjected to extraction with water and ethyl acetate. The ethyl acetate layer was taken, dried, and concentrated to obtain 4.8 g of light yellow solid with a yield of 96%. ESI-MS m/z calc. 198.0, found 198.9 (M+1)⁺.

### Step 3: 5-nitro-2,3-dihydrobenzo[d]isoxazol-3-one (5d)

THF (100 mL) and 5c (2.76 g) were added in a reaction flask. Triphenylphosphine (4.8 g) was added under nitrogen protection and stirred in an ice bath for 20 min. Then, DIAD (3.7 g) was added to react for 4 h, until the completion of the reaction was detected by LCMS. The reaction system was concentrated to remove the solvent, with water added, adjusted with sodium hydroxide until pH was 13, and subjected to extraction with DCM. The aqueous layer was taken, adjusted with hydrochloric acid until pH was 1, subjected to extraction with DCM, washed with saturated saline, and concentrated to obtain 1.6 g of white solid with a yield of 64%. ESI-MS m/z calc. 180.0, found 181.0 (M+1)⁺.

### Step 4: 5-amino-2,3-dihydrobenzo[d]isoxazol-3-one (5e)

Methanol (50 mL), 5d (800 mg), and 5% palladium on carbon (200 mg) were added in a reaction flask to react under hydrogenation conditions for 36 h, until the completion of the reaction was detected by LCMS. The reaction system was filtered. The filtrate was taken and concentrated to obtain 400 mg of white solid with a yield of 60.6%. ESI-MS m/z calc. 150.0, found 151.0 (M+1)⁺.

### Step 5: rel-(2R,3S,4S,5R)-3-(3,4-difluoro-2-methoxyphenyl)-4,5-dimethyl-N-(3-oxo-2H-benzo[d]isoxazol-5-yl)-5-(trifluoromethyl)tetrahydrofuran-2-carboxamide (Example 5)

Example 5 was obtained by a method similar to that in Example 1 with 5e that replaced 1b in Example 1. ESI-MS m/z calc. 486.1, found 487.1 (M+1)⁺. ¹H NMR (400 MHz, DMSO-d₆) δ 11.63 (s, 1H), 10.30 (s, 1H), 7.58 (d, J=2.0 Hz, 1H), 7.29-7.09 (m, 5H), 5.08 (d, J=10.4 Hz, 1H), 4.36-4.23 (m, 1H), 3.99 (d, J=2.1 Hz, 3H), 2.87-2.73 (m, 1H), 1.63 (s, 3H), 0.80-0.73 (m, 3H).

### Example 6: rel-(2R,3S,4S,5R)-3-(3,4-difluoro-2-methoxyphenyl)-4,5-dimethyl-N-(1-oxo-1,2,3,4-tetrahydroisoquinolin-7-yl)-5-(trifluoromethyl)tetrahydrofuran-2-carboxamide (6)

Example 6 was obtained by a method similar to that in Example 1 with 7-amino-1,2,3,4-tetrahydroisoquinolin-1-one that replaced 1b in Example 1. ESI-MS m/z calc. 498.2, found 499.2 (M+1)⁺. ¹H NMR (400 MHz, DMSO-d₆) δ 10.34 (s, 1H), 8.11 (d, J=2.3 Hz, 1H), 7.94 (d, J=3.3 Hz, 1H), 7.75 (dd, J=8.2, 2.4 Hz, 1H), 7.31-7.14 (m, 3H), 5.09 (d, J=10.4 Hz, 1H), 4.28 (dd, J=10.4, 7.6 Hz, 1H), 3.99 (d, J=2.1 Hz, 3H), 3.37 (td, J=6.5, 2.7 Hz, 2H), 2.87 (t, J=6.6 Hz, 2H), 2.82-2.76 (m, 1H), 1.64 (s, 3H), 0.83-0.71 (m, 3H).

### Example 8: rel-(2R,3S,4S,5R)-3-(3,4-difluoro-2-methoxyphenyl)-4,5-dimethyl-N-(1-oxo-2,3-dihydro-1H-isoindol-5-yl)-5-(trifluoromethyl)tetrahydrofuran-2-carboxamide

Example 8 was obtained by a method similar to that in Example 1 with 5-amino-2,3-dihydro-1H-isoindol-1-one that replaced 1b in Example 1. ESI-MS m/z calc. 484.1, found 485.2 (M+1)⁺. ¹H NMR (400 MHz, DMSO-d₆) δ 10.54 (s, 1H), 8.45 (s, 1H), 7.98 (d, J=6.5 Hz, 2H), 7.63 (d, J=1.1 Hz, 2H), 7.31-7.12 (m, 3H), 5.14 (d, J=10.3 Hz, 1H), 4.35 (s, 2H), 3.99 (d, J=2.1 Hz, 3H), 2.93 (s, 2H), 2.77 (s, 2H), 1.64 (s, 3H), 0.78 (d, J=7.4 Hz, 3H).

### Example 9

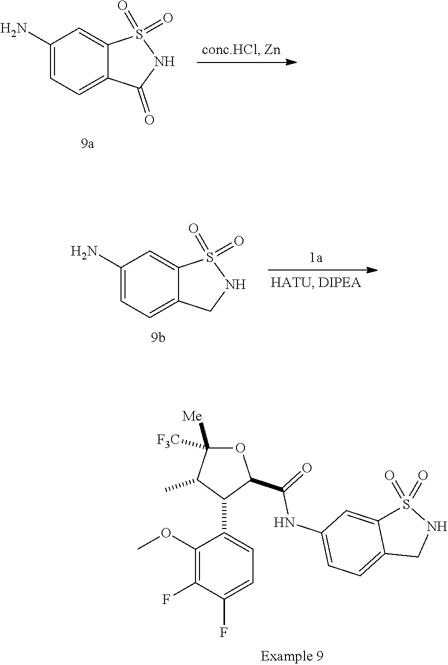

### Step 1: 6-aminophenylpropyl[d]isothiazol-3(2H)-one-1,2-dioxygen (9b)

9a (650 mg) and concentrated hydrochloric acid (9 mL) were added in a reaction flask in an ice bath, and zinc powder (1.1 g) was added in batches within about 30 min, and stirred at room temperature for 2 h. The reaction was monitored by LCMS until the raw material was fully converted. The reaction solution was adjusted with a saturated sodium bicarbonate solution in an ice bath to be weakly basic, and subjected to extraction with ethyl acetate (50 mL×3). The organic phases were combined, dried over anhydrous sodium sulfate, and filtered, to obtain 320 mg of brown solid (about 70% purity) with a yield of 37.0%. LCMS: MS m/z (254 nm): 185.0 [M+H]⁺.

### Step 2: rel-(2R,3S,4S,5R)-3-(3,4-difluoro-2-methoxyphenyl)-N-(1,1-dioxo-2,3-dihydrobenzo[d]isothiazol-6-yl)-4,5-dimethyl-5-(trifluoromethyl)tetrahydrofuran-2-carboxamide (Example 9)

Example 9 was obtained by a method similar to that in Example 3 with 9b that replaced 3b in Example 3. LCMS: Rt: 2.625 min; MS m/z (254 nm): 521.1 [M+H]⁺. ¹H NMR (400 MHz, DMSO-d6) δ 10.53 (s, 1H), 8.12 (d, J=1.5 Hz, 1H), 7.82 (t, J=4.7 Hz, 1H), 7.77 (dd, J=8.4, 1.8 Hz, 1H), 7.50 (d, J=8.4 Hz, 1H), 7.16 (t, J=6.5 Hz, 2H), 5.09 (d, J=10.3 Hz, 1H), 4.34 (d, J=4.8 Hz, 2H), 4.26 (dd, J=10.1, 7.8 Hz, 1H), 3.95 (d, J=2.0 Hz, 3H), 2.78 (dd, J=14.7, 7.4 Hz, 1H), 1.61 (s, 3H), 0.73 (d, J=6.7 Hz, 3H).

### Example 10: rel-(2R,3S,4S,5R)-3-(3,4-difluoro-2-methoxyphenyl)-N-(1,3-dioxo-2,3-dihydro-1H-isoindol-5-yl)-4,5-dimethyl-5-(trifluoromethyl)tetrahydrofuran-2-carboxamide (10)

Example 10 was obtained by a method similar to that in Example 1 with 5-amino-2H-isoindol-1,3-dione that replaced 1b in Example 1. ESI-MS m/z calc. 498.1, found 499.2 (M+1)⁺. ¹H NMR (400 MHz, DMSO-d₆) δ 11.24 (s, 1H), 10.73 (s, 1H), 8.14 (d, J=1.8 Hz, 1H), 7.94 (dd, J=8.2, 1.8 Hz, 1H), 7.77 (d, J=8.2 Hz, 1H), 7.27-7.11 (m, 2H), 5.11 (d, J=10.1 Hz, 1H), 4.32-4.16 (m, 1H), 3.95 (d, J=2.1 Hz, 3H), 2.85-2.71 (m, 1H), 1.61 (s, 3H), 0.74 (d, J=7.4 Hz, 3H).

### Example 12: rel-(2R,3S,4S,5R)-3-(3,4-difluoro-2-methoxyphenyl)-N-(1,4-dioxo-2,3-dihydrobenzo[2,1-d][1,2]dioxine-6-yl)-4,5-dimethyl-5-(trifluoromethyl)tetrahydrofuran-2-carboxamide (12)

Example 12 was obtained by a method similar to that in Example 1 with 6-amino-1,2,3,4-tetrahydrobenzo[2,1-d][1,2]dioxine-1,4-dione that replaced 1b in Example 1. ESI-MS m/z calc. 513.1, found 514.1 (M+1)⁺. ¹H NMR (400 MHz, DMSO-d₆) δ 11.52 (s, 1H), 10.76 (s, OH), 8.45 (s, OH), 8.07 (s, 1H), 7.21 (dd, J=8.6, 5.0 Hz, 1H), 5.16 (d, J=10.2 Hz, OH), 4.32 (dd, J=10.3, 7.6 Hz, 1H), 3.98 (dd, J=9.9, 2.1 Hz, 2H), 2.83 (q, J=7.6 Hz, OH), 1.66 (s, 2H), 0.83-0.62 (m, 2H).

### Example 13

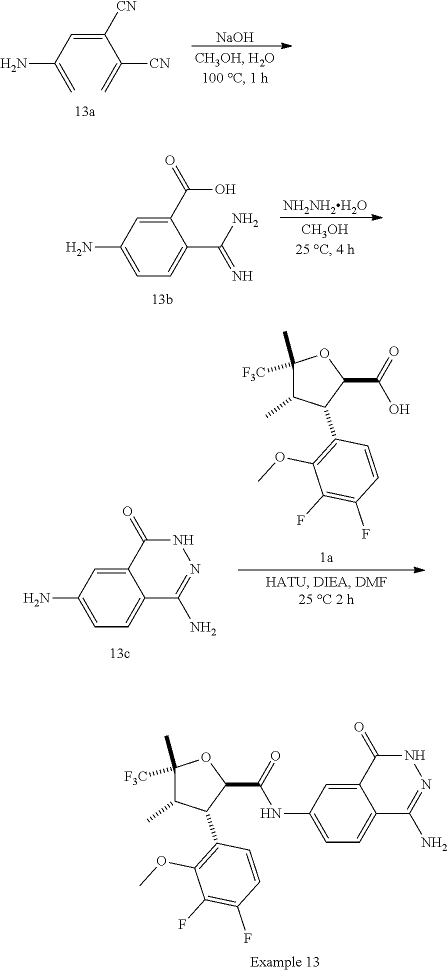

### Step 1: 5-amino-2-carbamimidoylbenzoic acid (13b)

The compound 4-aminophthalonitrile 13a (500 mg, 3.49 mmol, 1.0 eq) was dissolved in methanol and water in 3:2 (5 mL), and sodium hydroxide (139 mg, 3.49 mmol, 1.0 eq) was added. The reaction solution reacted at 100° C. under nitrogen protection for 1 h. After the completion of the reaction of the raw material was monitored by LCMS, the reaction solution was cooled down to room temperature and concentrated. The residue was separated and purified by prep-HPLC (0.01% FA) to obtain the compound 5-amino-2-carbamimidoylbenzoic acid 13b (733 mg, 82% yield), which was a yellow solid. LCMS: [M+H]⁺=180.1

### Step 2: 4,7-diaminophthalazin-1-ol (13c)

The compound 5-amino-2-carbamimidoylbenzoic acid 13b (680 mg, 3.79 mmol, 1.0 eq) was dissolved in methanol (10 mL), and hydrazine hydrate (7 mL) was added. The reaction solution reacted at 25° C. for 4 h. After the completion of the reaction was monitored by LCMS, the reaction mixture was concentrated. The residue was subjected to extraction with water (10 mL) and ethyl acetate (10 mL×5), washed with water and saturated saline sequentially, dried over anhydrous sodium sulfate, and then concentrated. The residue was filtered. The filter residue was dried under vacuum to obtain the compound 4,7-diaminophthalazin-1-ol 13c (100 mg, 13% yield), which was a yellow solid. LCMS: [M+H]⁺=177.1

### Step 3: rel-(2R,3S,4S,5R)—N-(1-amino-4-oxo-3H-benzo[2,1-d][1,2]dioxine-6-yl)-3-(3,4-difluoro-2-methoxyphenyl)-4,5-dimethyl-5-(trifluoromethyl)tetrahydrofuran-2-carboxamide (Example 13)

The compound 1a (50 mg, 0.14 mmol, 1.0 eq) was dissolved in N,N-dimethylformamide (2 mL), and benzotriazole-N,N,N′,N′-tetramethyluronium hexafluorophosphate (64 mg, 0.17 mmol, 1.2 eq), N,N-diisopropylethylamine (27 mg, 0.21 mmol, 1.5 eq), and 13c (29 mg, 0.17 mmol, 1.2 eq) were added. The reaction solution reacted at 25° C. for 2 h. After the completion of the reaction was monitored by LCMS, the reaction mixture was dissolved in water (10 mL), subjected to extraction with ethyl acetate (10 mL×3), washed with water and saturated saline sequentially, dried over anhydrous sodium sulfate, and then concentrated. The residue was purified by preparative separation (0.01% aqueous ammonia) to obtain the compound 13 (21.41 mg, 29% yield), which was a white solid. LCMS: [M+H]⁺=513.1. ¹H NMR (400 MHz, DMSO-d₆) δ 11.43 (s, 1H), 10.69 (s, 1H), 8.49 (d, J=2.0 Hz, 1H), 8.05 (dd, J=8.8, 2.4 Hz, 1H), 7.99 (d, J=8.8 Hz, 1H), 7.21-7.16 (m, 2H), 5.88 (s, 2H), 5.12 (d, J=10.0 Hz, 1H), 4.30-4.25 (m, 1H), 3.96 (d, J=2.0 Hz, 3H), 2.82-2.74 (m, 1H), 1.62 (s, 3H), 0.74 (d, J=6.4 Hz, 3H). ¹⁹F NMR (377 MHz, DMSO-d₆) δ −73.36 (s, 3H), −138.16 (d, J=20.7 Hz, 1H), −154.96 (d, J=20.7 Hz, 1H).

### Example 16: rel-2-carbamoyl-4-((2R,3S,4S,5R)-3-(3,4-difluoro-2-methoxyphenyl)-4,5-dimethyl-5-(trifluoromethyl)tetrahydrofuran-2-carboxamido)-1-methylpyridin-1-onium iodide

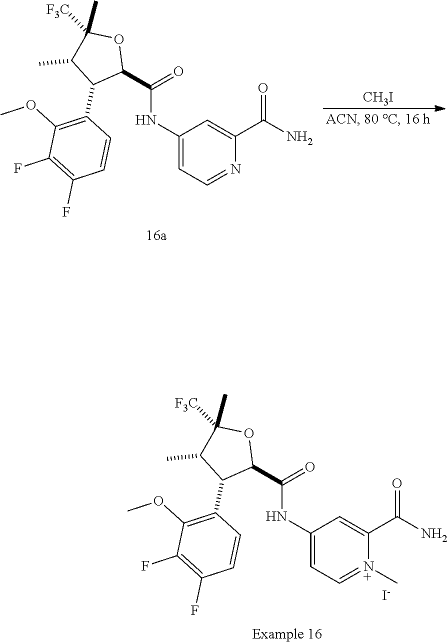

The compound 16a (40 mg, 0.08 mmol, 1.0 eq), iodomethane (1 mL), and acetonitrile (2 mL) were added sequentially in a microwave tube. The reaction solution reacted at 80° C. under nitrogen protection for 16 h. After the completion of the reaction was monitored by LCMS, the reaction mixture was concentrated. The residue was purified by preparative separation (0.01% aqueous ammonia) to obtain the compound 16 (11.06 mg, 27% yield), which was a white solid. LCMS: [M]⁺=488.1. ¹H NMR (400 MHz, CDCl₃) δ 11.38 (s, 1H), 9.92 (s, 1H), 8.83 (s, 1H), 8.69 (s, 1H), 8.12 (d, J=12.8 Hz, 1H), 7.09-7.04 (m, 1H), 6.87-6.85 (m, 1H), 6.06 (s, 1H), 5.05 (d, J=11.2 Hz, 1H), 4.31 (s, 3H), 4.27-4.20 (m, 1H), 3.97 (d, J=2.6 Hz, 3H), 2.89-2.79 (m, 1H), 0.92 (s, 3H), 0.78 (d, J=6.4 Hz, 3H).

### Example 20

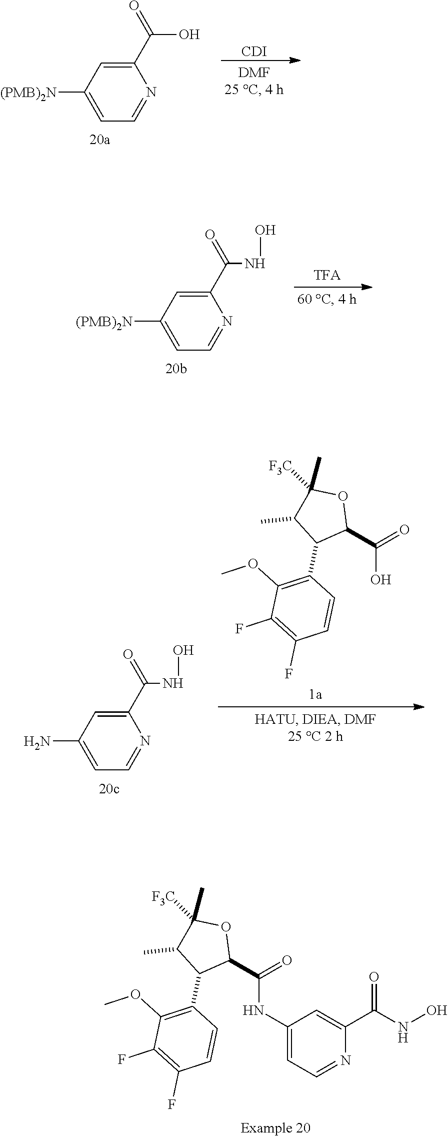

### Step 1: 4-(bis(4-methoxybenzyl)amino)-N-hydroxypicolinamide (20b)

The compound 4-(bis(4-methoxybenzyl)amino)picolinic acid 20a (800 mg, 2.1 mmol, 1.0 eq) was dissolved in N,N-dimethylformamide (10 mL), and N,N′-carbonyldiimidazole (411.32 mg, 2.5 mmol, 1.2 eq) was added and stirred at 25° C. for 2 h. Then, hydroxylamine hydrochloride (882.81 mg, 10.6 mmol, 5.0 eq) was added. The reaction solution was stirred at 25° C. to react for 2 h. After the completion of the reaction was monitored by LCMS, the reaction solution was poured into water and subjected to extraction with ethyl acetate. The organic phase was washed with saturated saline. The organic phase was dried and concentrated. The residue was separated and purified by silica gel column chromatography (dichloromethane/methanol=10/1) to obtain the compound 20b (500 mg, 60% yield), which was a brown solid. LCMS: [M+H]⁺=394.2.

### Step 2: 4-amino-N-hydroxypicolinamide (20c)

The compound 20b (500 mg, 1.3 mmol, 1.0 eq) was dissolved in trifluoroacetic acid (3 mL). The reaction solution was stirred at 60° C. to react for 4 h. After the completion of the reaction was monitored by LCMS, the reaction solution was concentrated. The residue was dissolved in dichloromethane, and a hydrogen chloride ether solution (2 mL) was added, and concentrated to obtain a hydrochloride salt of the compound 20c (260 mg, 93% yield), which was a brown solid. LCMS: [M+H]⁺=154.2.

### Step 3: rel-4-(2R,3S,4S,5R)-3-(3,4-difluoro-2-methoxyphenyl)-4,5-dimethyl-5-trifluoromethyl tetrahydrofuran-2-carboxamide)-N-hydroxypicolinamide (Example 20)

The compound 1a (50 mg, 0.14 mmol, 1.0 eq) was dissolved in N,N-dimethylformamide (2 mL), and benzotriazole-N,N,N′,N′-tetramethyluronium hexafluorophosphate (80.48 mg, 0.21 mmol, 1.5 eq), 20c (64.8 mg, 0.42 mmol, 3.0 eq), and N,N-diisopropylethylamine (91.2 mg, 0.7 mmol, 5.0 eq) were added sequentially. The reaction solution was stirred at 25° C. to react for 2 h. After the completion of the reaction was monitored by LCMS, the reaction solution was poured into water and subjected to extraction with ethyl acetate. The organic phase was washed with saline. The organic phase was dried and concentrated. The residue was purified by preparative separation (0.01% formic acid) to obtain 20 (4.41 mg, 6% yield), which was a white solid.

LCMS: [M+H]⁺=490.1, ¹H NMR (400 MHz, CH₃OD) δ 7.89 (d, J=6.9 Hz, 1H), 7.26-7.16 (m, 2H), 6.97-6.90 (m, 1H), 6.79-6.64 (m, 1H), 5.17 (s, 1H), 4.33-4.27 (m, 1H), 4.01 (d, J=2.2 Hz, 3H), 2.84-2.77 (m, 1H), 1.64 (s, 3H), 0.80 (d, J=6.2 Hz, 3H).

### Example 21

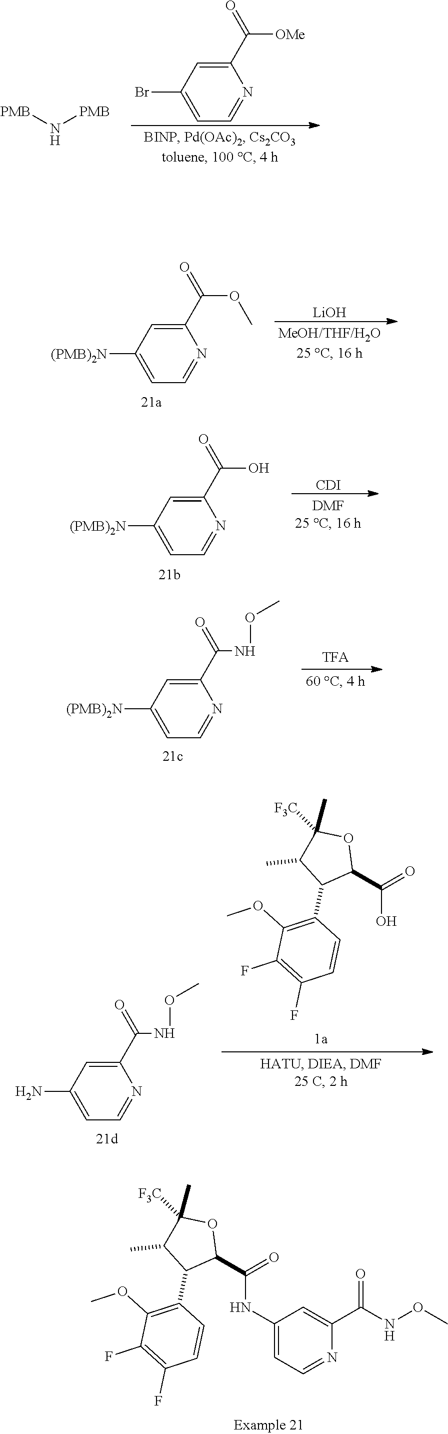

### Step 1: 4-(bis(4-methoxybenzyl)amino)methyl picolinate (21a)

Bis(4-methoxybenzyl)amine (4 g, 1.8 mmol, 1.0 eq), methyl 4-bromopicolinate (6.2 g, 2.4 mmol, 1.3 eq), 1,1′-[1,1′-binaphthyl]-2,2′-diylbis[1,1-diphenyl-(ACI)phosphine (580 mg, 0.09 mmol, 0.05 eq), palladium acetate (210 mg, 0.09 mmol, 0.05 eq), cesium carbonate (12.06 g, 3.7 mmol, 2.0 eq), and toluene (40 mL) were added sequentially in a microwave tube and stirred to react at 100° C. for 4 h. After the completion of the reaction was monitored by LCMS, the reaction solution was filtered, and the filtrate was concentrated. The residue was separated and purified by silica gel column chromatography (petroleum ether/ethyl acetate=1/1) to obtain 21a (3.2 g, 44% yield), which was a yellow solid. LCMS: [M+H]⁺=393.2.

### Step 2: 4-(bis(4-methoxybenzyl)amino)picolinic acid (21b)

The compound 4-(bis(4-methoxybenzyl)amino)methyl picolinate 21a (3.2 g, 8.2 mmol, 1.0 eq) was dissolved in methanol, tetrahydrofuran, and water in 3/3/1 (30 mL), and lithium hydroxide monohydrate (1 g, 24.6 mmol, 3.0 eq) was added and stirred to react at 25° C. for 16 h. After the completion of the reaction was monitored by LCMS, the reaction solution was poured into water, adjusted with 1 N dilute hydrochloric acid to pH 5, and subjected to extraction with ethyl acetate. The organic phase was washed with saline. The organic phase was dried and concentrated to obtain the compound 4-(bis(4-methoxybenzyl)amino)picolinic acid 21b (2 g, 65% yield), which was a yellow solid. LCMS: [M+H]⁺=379.1

### Step 3: 4-(bis(4-methoxybenzyl)amino)-N-methoxypicolinamide (21c)

The compound 4-(bis(4-methoxybenzyl)amino)picolinic acid 21b (200 mg, 0.53 mmol, 1.0 eq) was dissolved in N,N-dimethylformamide (2 mL), and N,N′-carbonyldiimidazole (102.8 mg, 0.63 mmol, 1.2 eq) was added and stirred at 25° C. for 2 h. Then, methoxyamine hydrochloride (221 mg, 10.6 mmol, 5.0 eq) was added. The reaction solution was stirred at room temperature to react for 2 h. After the completion of the reaction was monitored by LCMS, the reaction solution was poured into water and subjected to extraction with ethyl acetate. The organic phase was washed with saline. The organic phase was dried and concentrated. The residue was separated and purified by silica gel column chromatography (dichloromethane/methanol=20/1) to obtain the compound 4-(bis(4-methoxybenzyl)amino)-N-methoxypicolinamide 21c (140 mg, 65% yield), which was a brown solid. LCMS: [M+H]⁺=408.1

### Step 4: 4-amino-N-methoxypicolinamide (21d)

The compound 4-(bis(4-methoxybenzyl)amino)-N-methoxypicolinamide 21c (100 mg, 0.24 mmol, 1.0 eq) was dissolved in trifluoroacetic acid (2 mL). The reaction solution was stirred at 60° C. to react for 4 h. After the completion of the reaction was monitored by LCMS, the reaction solution was concentrated. The residue was dissolved in dichloromethane, and hydrogen chloride ether (2 mL) was added, and concentrated to obtain a hydrochloride salt of the compound 4-amino-N-methoxypicolinamide 21d (80 mg, 78% yield), which was a brown solid. LCMS: [M+H]⁺=168.1

### Step 5: rel-4-(2R,3S,4S,5R)-3-(3,4-difluoro-2-methoxyphenyl)-4,5-dimethyl-5-trifluoromethyl tetrahydrofuran-2-carboxamidopicolinamide (Example 21)

The compound 1a (50 mg, 0.14 mmol, 1.0 eq) was dissolved in N,N-dimethylformamide (2 mL), and benzotriazole-N,N,N′,N′-tetramethyluronium hexafluorophosphate (80.48 mg, 0.21 mmol, 1.5 eq), 4-amino-N-methoxypicolinamide 21d (70.7 mg, 0.42 mmol, 3.0 eq), and N,N-diisopropylethylamine (91.2 mg, 0.7 mmol, 5.0 eq) were added sequentially. The reaction solution was stirred at 25° C. to react for 2 h. After the completion of the reaction of most of the raw materials was monitored by LCMS, the reaction solution was poured into water and subjected to extraction with ethyl acetate. The organic phase was washed with saline. The organic phase was dried and concentrated. The residue was purified by preparative separation (0.01% formic acid) to obtain the compound 21 (4.5 mg, 6% yield), which was a white solid. LCMS: [M+H]⁺=504.1. ¹H NMR (400 MHz, CH₃OD) δ 8.53-8.43 (m, 1H), 8.23 (s, 1H), 7.88 (s, 1H), 7.10 (d, J=7.8 Hz, 1H), 7.02-6.93 (m, 1H), 5.06 (s, 1H), 4.37-4.29 (m, 1H), 3.99 (d, J=2.2 Hz, 3H), 3.80 (s, 3H), 2.83-2.75 (m, 1H), 1.65 (s, 3H), 0.81 (d, J=7.6 Hz, 3H).

### Example 22: rel-(2R,3S,4S,5R)—N-[2-(aminothiomethylidene)pyridin-4-yl]-3-(3,4-difluoro-2-methoxyphenyl)-4,5-dimethyl-5-(trifluoromethyl)tetrahydrofuran-2-carboxamide

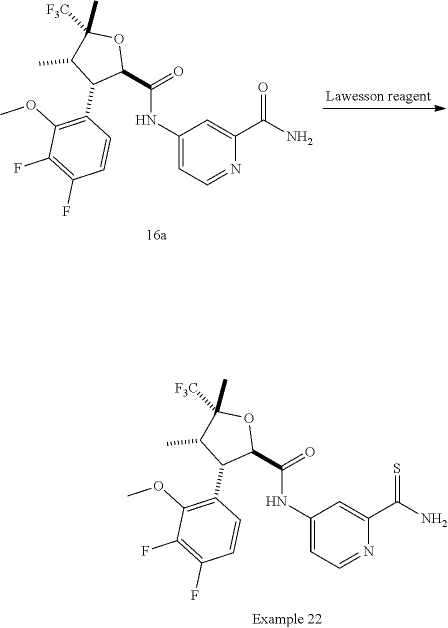

The compound 16a (30 mg, 0.06 mmol, 1.0 eq) was dissolved in toluene (20 mL), and the Lawesson reagent (26 mg, 0.06 mmol, 1.0 eq) was added, and heated to 110° C. to react for 24 h. After the reaction, the reaction system was concentrated to remove the solvent, and prepared by thin-layer chromatography, to obtain Example 22 (15 mg, 48% yield), which was a yellow solid. LCMS: [M+H]⁺=490.5. ¹H NMR (400 MHz, DMSO-d₆) δ 11.49 (s, 1H), 10.21 (s, 1H), 9.93 (s, 1H), 8.89-8.71 (m, 1H), 8.49 (d, J=5.4 Hz, 1H), 7.93 (s, 1H), 7.46-7.02 (m, 3H), 5.29 (d, J=10.7 Hz, 1H), 4.27 (d, J=8.9 Hz, 1H), 3.99 (d, J=2.8 Hz, 3H), 2.75 (m, 1H), 1.64 (s, 3H), 0.75 (s, 3H).

### Example 24: rel-(2R,3S,4S,5R)-3-(3,4-difluoro-2-methoxyphenyl)-N-(6-fluoro-2-oxo-1H-pyridin-4-yl)-4,5-dimethyl-5-(trifluoromethyl)tetrahydrofuran-2-carboxamide (24)

Example 24 was obtained by a method similar to that in Example 1 with 4-amino-6-fluoro-1,2-dihydropyridin-2-one that replaced 1b in Example 1. ESI-MS m/z calc. 464.1, found 465.1 (M+1)⁺. ¹H NMR (400 MHz, Methanol-d₄) δ 7.08 (t, J=7.5 Hz, 1H), 7.01-6.93 (m, 1H), 6.83 (s, 1H), 6.75 (d, J=1.4 Hz, 1H), 5.02 (d, J=10.3 Hz, 1H), 4.30 (dd, J=10.3, 8.0 Hz, 1H), 3.99 (d, J=2.2 Hz, 3H), 2.87-2.70 (m, 1H), 1.63 (s, 3H), 0.80 (dd, J=7.8, 2.4 Hz, 3H).

### Example 25

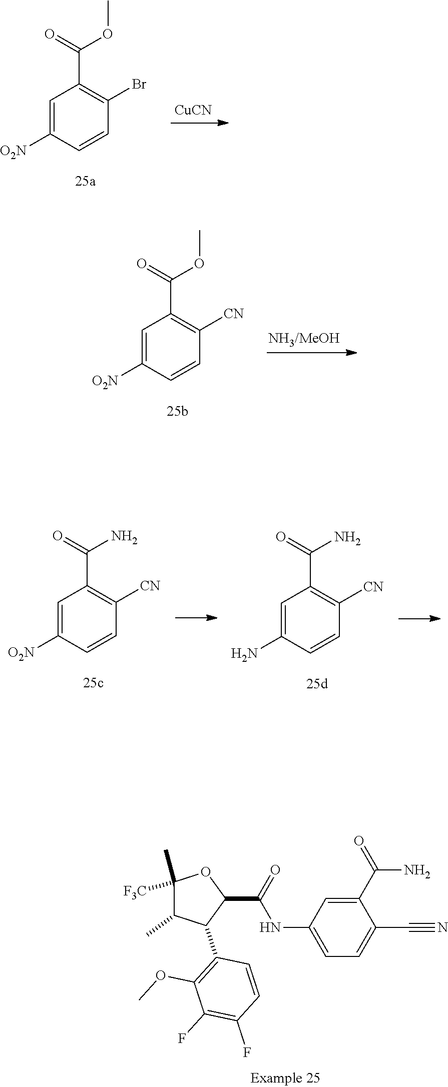

### Step 1: methyl 2-cyano-5-nitrobenzoate (25b)

25a (3 g), cuprous cyanide (2 g), and DMF (30 mL) were added in a reaction flask and stirred uniformly, and Pd₂(dba)₃ (527 mg) and dppf (638 mg) were added sequentially, with nitrogen displacement for three times, and stirred at 115° C. for 16 h. The reaction solution was cooled down to room temperature, with water (70 mL) added, and subjected to extraction with ethyl acetate (40 mL×3). The organic phases were combined, concentrated, and separated by column chromatography (EA/PE, 0%-15%), to obtain 2 g of white solid with a yield of 84.4%. LCMS: m/z (254 nm): 207.0 [M+H]⁺

### Step 2: 2-cyano-5-nitrobenzamide (25c)

25a (1 g) and an ammonia in methanol solution (7 M, 10 mL) were added in a reaction flask and stirred at room temperature for 16 h. The reaction solution was filtered and washed with methanol to obtain 720 mg of light yellow solid with a yield of 77.6%. LCMS: m/z (254 nm): 192.0 [M+H]⁺

### Step 3: 2-cyano-5-anthranilamide (25d)

25c (300 mg), MeOH (10 mL), and THF (10 mL) were added in a reaction flask and stirred uniformly. Then, palladium on carbon (30 mg) was added, with hydrogen displacement for three times, and stirred at room temperature for 6 h. The reaction was monitored by TLC until the raw material was fully converted. The reaction solution was filtered with diatomaceous earth. The filtrate was concentrated to obtain 179 mg of yellow solid with a yield of 71%. LCMS: m/z (254 nm): 162.0 [M+H]⁺. ¹H NMR (400 MHz, DMSO-d6) δ 9.00 (s, 2H), 7.58 (d, 1H), 6.77 (d, 1H), 6.73 (dd, 1H), 5.99 (s, 2H).

### Step 4: rel-(2R,3S,4S,5R)—N-(3-carbamoyl-4-cyanophenyl)-3-(3,4-difluoro-2-methoxyphenyl)-4,5-dimethyl-5-(trifluoromethyl)tetrahydrofuran-2-carboxamide (Example 25)

Example 25 was obtained by a method similar to that in Example 3 with 25d that replaced 3b in Example 3.

¹H NMR (400 MHz, DMSO) δ 10.87 (s, 1H), 7.26-7.20 (m, 1H), 7.18 (d, J=8.4 Hz, 1H), 7.08 (dd, J=17.3, 9.3 Hz, 1H), 6.85 (d, J=2.0 Hz, 1H), 6.72 (dd, J=8.4, 2.0 Hz, 1H), 6.39 (s, 2H), 5.22 (d, J=10.6 Hz, 1H), 4.23 (dd, J=10.5, 7.5 Hz, 1H), 3.90 (d, J=2.0 Hz, 3H), 2.72-2.61 (m, 1H), 1.51 (s, 3H), 0.71 (d, J=6.2 Hz, 3H).

### Example 27: rel-(2R,3S,4S,5R)-3-(3,4-difluoro-2-methoxyphenyl)-4,5-dimethyl-N-(6-oxo-1H-1,2-dioxine-3-yl)-5-(trifluoromethyl)tetrahydrofuran-2-carboxamide (27)

Example 27 was obtained by a method similar to that in Example 1 with 6-amino-3H,2H-1,2-dioxine-3-one that replaced 1b in Example 1. ESI-MS m/z calc. 447.1, found 448.1 (M+1)⁺. ¹H NMR (400 MHz, DMSO-d₆) δ 12.79 (s, 1H), 10.75 (s, 1H), 7.88 (d, J=10.1 Hz, 1H), 7.28-7.11 (m, 2H), 6.92 (d, J=10.1 Hz, 1H), 5.14 (d, J=10.4 Hz, 1H), 4.30-4.20 (m, 1H), 3.98 (t, J=2.5 Hz, 3H), 2.78 (t, J=7.5 Hz, 1H), 1.62 (s, 3H), 0.80-0.68 (m, 2H).

### Example 29: rel-(2R,3S,4S,5R)-2-({[3-(3,4-difluoro-2-methoxyphenyl)-4,5-dimethyl-5-(trifluoromethyl)tetrahydrofuran-2-yl]carbonyl}amino)-1,3-thiazol-4-carboxamide (29)

Example 29 was obtained by a method similar to that in Example 3 with 2-amino-1,3-thiazol-4-carboxamide that replaced 3b in Example 3. ESI-MS m/z calc. 479.1, found 480.1 (M+1)⁺. ¹H NMR (400 MHz, DMSO-d₆) δ 12.63 (s, 1H), 7.86 (s, 1H), 7.59 (s, 1H), 7.37 (s, 1H), 7.26-7.15 (m, 2H), 5.25 (d, J=10.5 Hz, 1H), 4.30 (dd, J=10.4, 7.6 Hz, 1H), 3.98 (d, J=2.2 Hz, 2H), 2.82 (t, J=7.5 Hz, 1H), 1.64 (s, 2H), 0.80-0.68 (m, 3).

### Example 30: rel-(2R,3S,4S,5R)-4-({[3-(3,4-difluoro-2-methoxyphenyl)-4,5-dimethyl-5-(trifluoromethyl)tetrahydrofuran-2-yl]carbonyl}amino)benzene-1,2-dicarboxamide (30)

Example 30 was obtained by a method similar to that in Example 1 with 4-aminobenzene-1,2-dicarboxamide that replaced 1b in Example 1. ESI-MS m/z calc. 515.1, found 516.1 (M+1)⁺. ¹H NMR (400 MHz, DMSO-d₆) δ 10.42 (s, 1H), 7.76-7.60 (m, 4H), 7.49 (d, J=8.2 Hz, 1H), 7.30 (d, J=20.8 Hz, 2H), 7.22-7.09 (m, 2H), 5.07 (d, J=10.4 Hz, 1H), 4.24 (dd, J=10.3, 7.6 Hz, 1H), 3.95 (d, J=2.1 Hz, 2H), 2.76 (t, J=7.5 Hz, 1H), 1.60 (s, 3H), 0.73 (d, J=7.4 Hz, 3H).

Example 30 was subjected to chiral resolution by SFC with a column: Daicel CHIRALPAK IH_3, 3.0×150 mm, 3 μm particle size, to obtain Example 30-1 (RT=1.6 min) with specific rotation

$${[ α ]}_{D}^{20} = 35.4 ,$$

and Example 30-2 (RT=3.0 min) with specific rotation

$${[ α ]}_{D}^{20} = - 35.5 .$$

### Example 31: rel-(2R,3S,4S,5R)-5-({[3-(3,4-difluoro-2-methoxyphenyl)-4,5-dimethyl-5-(trifluoromethyl)tetrahydrofuran-2-yl]carbonyl}amino)benzene-1,3-dicarboxamide

Example 31 was obtained by a method similar to that in Example 1 with 5-aminobenzene-1,3-dicarboxamide that replaced 1b in Example 1. ESI-MS m/z calc. 515.1, found 516.1 (M+1)⁺. ¹H NMR (400 MHz, DMSO-d₆) δ 10.50 (s, 1H), 8.23 (d, J=1.5 Hz, 2H), 8.10 (t, J=1.6 Hz, 1H), 7.99 (d, J=3.2 Hz, 2H), 7.47 (s, 2H), 7.26-7.20 (m, 2H), 5.14 (d, J=10.5 Hz, 1H), 4.29 (dd, J=10.5, 7.6 Hz, 1H), 3.99 (d, J=2.1 Hz, 3H), 2.93 (s, 1H), 1.65 (s, 3H), 0.77 (d, J=7.4 Hz, 3H).

### Example 32

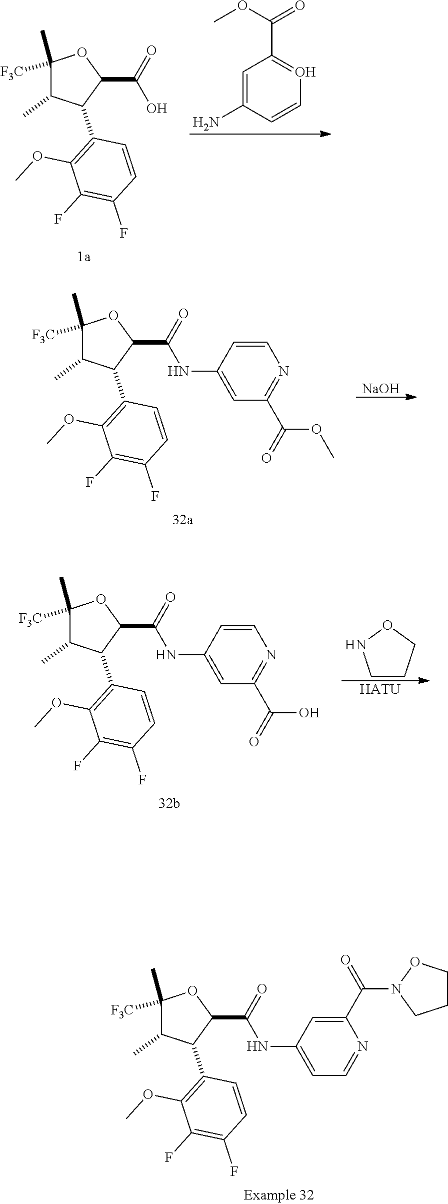

### Step 1: rel-(2R,3S,4S,5R)-4-({[3-(3,4-difluoro-2-methoxyphenyl)-4,5-dimethyl-5-(trifluoromethyl)tetrahydrofuran-2-yl]carbonyl}amino)methyl picolinate (32a)

The intermediate 32a was obtained by a method similar to that in Example 3 with methyl 4-aminopyridine-2-carboxylate that replaced 3a in Example 3. ESI-MS m/z calc. 488.1, found 489.1 (M+1)⁺.

### Step 2: rel-(2R,3S,4S,5R)-4-({[3-(3,4-difluoro-2-methoxyphenyl)-4,5-dimethyl-5-(trifluoromethyl)tetrahydrofuran-2-yl]carbonyl}amino)2-picolinic acid (32b)

32a (80 mg), THF (5 mL), water (5 mL), and sodium hydroxide (10 mg) were added in a reaction flask to react at room temperature for 2 h, until the completion of the reaction was monitored by TLC. The reaction system was adjusted to pH 1 and subjected to extraction with ethyl acetate. The organic phase was dried, concentrated, and then purified by column chromatography, to obtain 32b (75 mg, 98%). ESI-MS m/z calc. 474.1, found 473.1 (M−1)⁺.

### Step 3: rel-(2R,3S,4S,5R)-3-(3,4-difluoro-2-methoxyphenyl)-4,5-dimethyl-N-[2-(tetrahydroisoxazol-2-ylcarbonyl)pyridin-4-yl]-5-(trifluoromethyl)tetrahydrofuran-2-carboxamide (Example 32)

32b (30 mg), tetrahydroisoxazole (10 mg), HATU (36 mg), diisopropylethylamine (20 mg), and DMF (1 mL) were added in a reaction flask to react at room temperature for 17 h, until the completion of the reaction was monitored by LCMS. Water and ethyl acetate were added to carry out extraction. The organic phase was dried, concentrated, and then purified by thin-layer chromatography, to obtain Example 32 (20 mg, 60%). ESI-MS m/z calc. 529.1, found 530.2 (M+1)⁺. ¹H NMR (400 MHz, DMSO-d₆) δ 10.74 (s, 1H), 8.51 (d, J=5.6 Hz, 1H), 8.02 (d, J=27.4 Hz, 1H), 7.76 (s, 1H), 7.20 (dd, J=9.7, 6.5 Hz, 2H), 5.14 (d, J=10.2 Hz, 1H), 4.29 (dd, J=10.2, 7.7 Hz, 1H), 3.98 (d, J=2.2 Hz, 4H), 3.91 (d, J=14.6 Hz, OH), 2.85 (d, J=63.6 Hz, 2H), 2.81 (t, J=7.5 Hz, 1H), 2.32 (p, J=7.0 Hz, 2H), 1.64 (s, 3H), 0.77 (d, J=7.4 Hz, 3H).

### Example 34

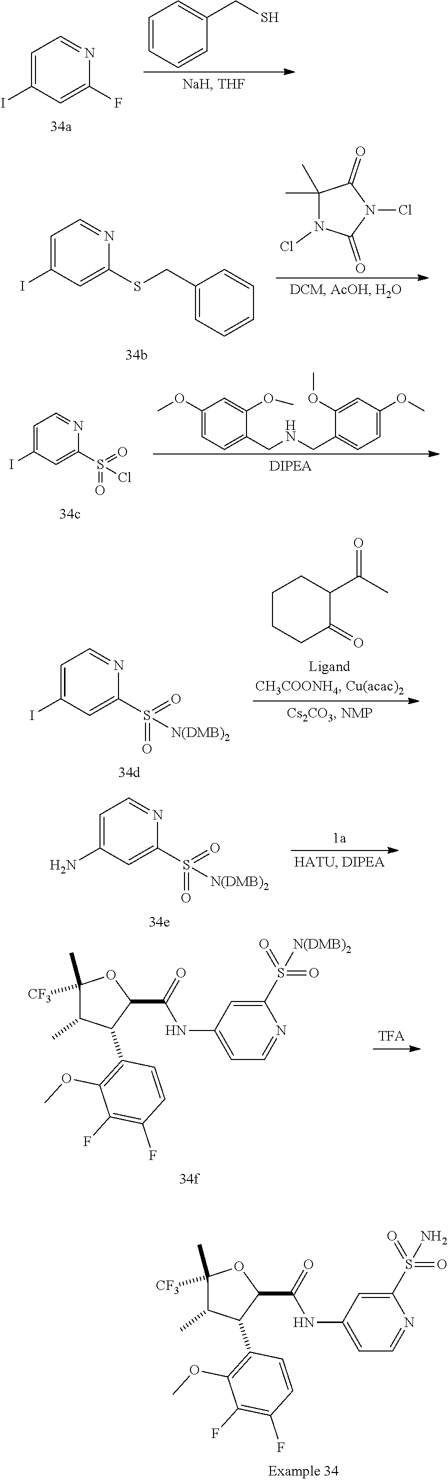

### Step 1: 2-benzylthio-4-iodopyridine (34b)

Benzyl mercaptan (2.9 g) and THF (50 mL) were added in a reaction flask and stirred uniformly, and NaH (848 mg) was added in batches in an ice bath, and then stirred at room temperature for 1 h. 34a (5 g) was added in an ice bath, and then stirred at room temperature for 3 h. The reaction was monitored by LCMS until the raw material was fully converted. The reaction solution with water (10 mL) added was subjected to extraction with ethyl acetate (10 mL×3). The organic phases were combined, dried over anhydrous sodium sulfate, filtered, concentrated, and separated by column chromatography (EA/PE, 0%-10%), to obtain 6.6 g of light red liquid with a yield of 90.1%. LCMS: MS m/z (254 nm): 327.9 [M+H]⁺.

### Step 2: 4-iodopyridine-2-sulfonyl chloride (34c)

34b (3 g), DCM (30 mL), and DIPEA (3.8 g) were added in a reaction flask, and 1,3-dichloro-5,5-dimethylhydantoin (6.9 g) was added in batches in an ice bath, and then warmed to room temperature and stirred for 4 h, until the completion of the reaction was monitored by LCMS. The reaction solution with water (50 mL) added was subjected to extraction with dichloromethane (20 mL×3). The organic phases were combined, dried over anhydrous sodium sulfate, filtered, concentrated, and separated by column chromatography (EA/PE, 0%-10%), to obtain 1.4 g of light yellow solid with a yield of 24.2%. LCMS: MS m/z (254 nm): 585.0 [M+H]⁺.

### Step 3: N,N-bis(2,4-dibenzyl)-4-iodopyridine-2-sulfonamide (34d)

34c (800 mg), N-methylpyrrolidone (7 mL), ammonium acetate (1.06 g), copper acetylacetonate (107 mg), Ligand (115 mg), and cesium carbonate (1.34 g) were added in a reaction flask, with nitrogen displacement for three times, and stirred at 95° C. for 16 h. The reaction solution was cooled down to room temperature, with water (20 mL) added, and subjected to extraction with ethyl acetate (20 mL×3). The organic phases were combined, concentrated, and separated by column chromatography (EA/PE, 50%-100%), to obtain 460 mg of yellow oily substance with a yield of 70.8%. LCMS: MS m/z (254 nm): 475.1 [M+H]⁺.

### Step 4: rel-(2R,3S,4S,5R)—N-(2-(N,N-bis(2,4-dimethoxybenzyl)sulfonyl)pyridin-4-yl)-3-(3,4-difluoro-2-methoxyphenyl)-4,5-dimethyl-5-(trifluoromethyl)tetrahydrofuran-2-carboxamide (34e)

34e was obtained by a method similar to that in Example 3 with 34d that replaced 3b in Example 3. LCMS: MS m/z (254 nm): 810.2 [M+H]⁺.

### Step 5: rel-(2R,3S,4S,5R)-3-(3,4-difluoro-2-methoxyphenyl)-4,5-dimethyl-N-(2-sulfonylpyridin-4-yl)-5-(trifluoromethyl)tetrahydrofuran-2-carboxamide (Example 34)

34e (52 mg), DCM (2 mL), and trifluoroacetic acid (146 mg) were added in a reaction flask and stirred at room temperature for 12 h. The reaction was monitored by LCMS until the raw material was fully converted. The reaction solution was concentrated and separated by silica gel plate chromatography (EA/PE, 40%) to obtain 24.5 mg of white solid with a yield of 75.2%. LCMS: MS m/z (254 nm): 510.1 [M+H]⁺. ¹H NMR (400 MHz, DMSO-d6) (¹H NMR (400 MHz, DMSO-d6) δ 10.84 (s, 1H), 8.57 (d, J=5.4 Hz, 1H), 8.26 (d, J=1.8 Hz, 1H), 7.81 (dd, J=5.5, 1.7 Hz, 1H), 7.41 (s, 2H), 7.23-7.11 (m, 2H), 5.12 (d, J=10.1 Hz, 1H), 4.36-4.15 (m, 1H), 3.95 (d, J=1.8 Hz, 3H), 2.93-2.66 (m, 1H), 1.61 (s, 3H), 0.73 (d, J=6.1 Hz, 3H).

### Example 59

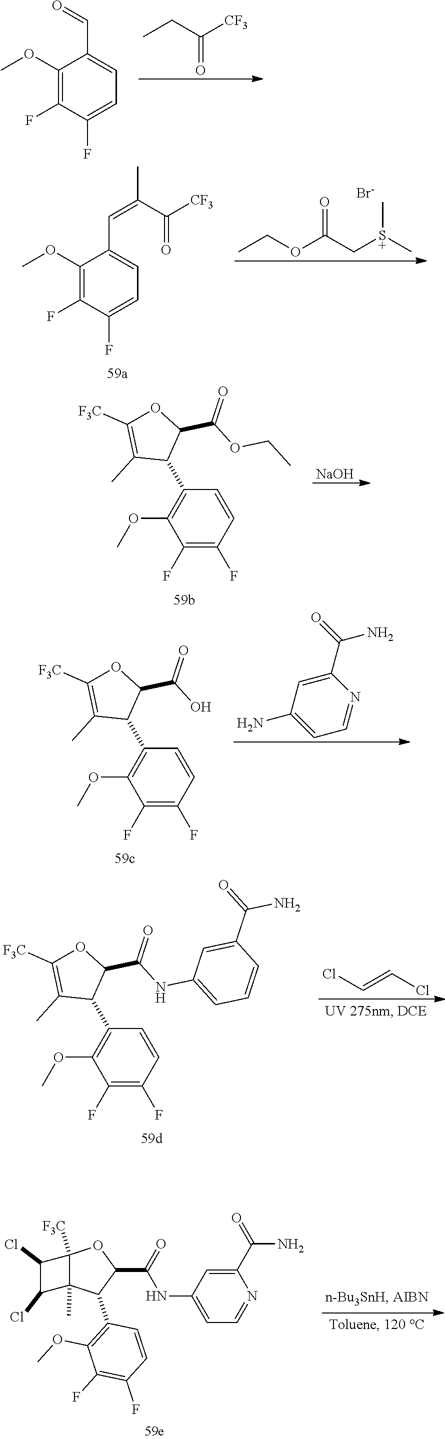

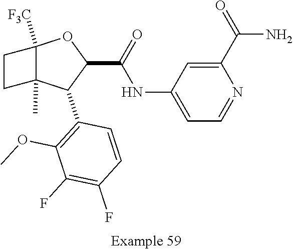 [SMILES: COc1c([C@H]2[C@H](C(=O)Nc3ccnc(C(N)=O)c3)O[C@]3(C(F)(F)F)CC[C@]23C)ccc(F)c1F]

### Step 1: (3Z)-4-(3,4-difluoro-2-methoxyphenyl)-1,1,1-trifluoro-3-methyl-3-buten-2-one (59a)

Toluene (25 mL), 3,4-difluoro-2-methoxybenzaldehyde (5.0 g, 29.0 mmol, 1.0 eq), 1,1,1-trifluorobutan-2-one (11 g, 87.2 mmol, 3.0 eq), piperidinium acetate (4.2 g, 29.0 mmol, 1.0 eq), and acetic acid (0.9 g, 14.5 mmol, 0.5 eq) were added sequentially in a reaction flask to react with the reaction flask sealed at room temperature for 2 h, and heated to 75° C. to react for 16 h, until the completion of the reaction was monitored by TLC. Extraction was carried out with water and ethyl acetate. The ethyl acetate layer was taken, concentrated, and then separated by column chromatography (PE:EA=20:1), to obtain 59a (7.5 g, 92% yield), which was a yellow liquid.

### Step 2: rel-(2R,3S)-3-(3,4-difluoro-2-methoxyphenyl)-4-methyl-5-(trifluoromethyl)-2,3-dihydrofuran-2-carboxylic acid ethyl ester (59b)

Acetonitrile (25 mL), the compound 59a (2.5 g, 8.9 mmol, 1.0 eq), (ethoxycarbonylmethyl)dimethylsulfonium bromide (2.6 g, 11.6 mmol, 1.3 eq), and cesium carbonate (3.8 g, 11.6 mmol, 1.3 eq) were added sequentially in a reaction flask to react at room temperature for 16 h, until the completion of the reaction of the compound 59a was monitored by TLC (PE:EA=10:1). The reaction system was filtered. The filtrate was taken, concentrated, and then separated by column chromatography (PE:EA=20:1), to obtain 2.3 g of yellow liquid (72% yield).

### Step 3: rel-(2R,3S)-3-(3,4-difluoro-2-methoxyphenyl)-4-methyl-5-(trifluoromethyl)-2,3-dihydrofuran-2-carboxylic acid (59c)

Methanol (25 mL), water (25 mL), the compound 59b (1.0 g, 2.7 mmol, 1.0 eq), and sodium hydroxide (0.54 g, 13.6 mmol, 5 eq) were added sequentially in a reaction flask to react at 65° C. for 16 h, until the completion of the reaction was monitored by LCMS. The reaction system was concentrated to remove methanol, adjusted with 1 N hydrochloric acid to be acidic, and subjected to extraction with ethyl acetate. The ethyl acetate layer was taken, dried, and concentrated to obtain 59c (0.8 g, 86% yield), which was a yellow liquid.

### Step 4: rel-(2R,3S)-4-({[3-(3,4-difluoro-2-methoxyphenyl)-4-methyl-5-(trifluoromethyl)-2,3-dihydrofuran-2-yl]carbonyl}amino)pyridin-2-carboxamide (59d)

DMF (20 mL), 59c (0.8 g, 2.3 mmol, 1.0 eq), HATU (1.4 g, 3.5 mmol, 1.5 eq), triethylamine (0.7 g, 4.7 mmol, 2.0 eq), and 4-aminopyridine-2-carboxamide (0.5 g, 3.5 mmol, 1.5 eq) were added sequentially in a reaction flask to react at room temperature for 16 h, until the completion of the reaction was monitored by TLC. Extraction was carried out with water and ethyl acetate. The ethyl acetate layer was taken, concentrated, and then separated by column chromatography (PE:EA=3:1-1:2), to obtain 59d (0.4 g, 40% yield), which was a gray solid. MS: [M+H]⁺=458. ¹H NMR (400 MHz, DMSO-d₆) δ 10.77 (s, 1H), 8.56 (d, J=5.5 Hz, 1H), 8.33 (d, J=2.1 Hz, 1H), 8.11 (s, 1H), 7.86 (dd, J=5.6, 2.3 Hz, 1H), 7.66 (s, 1H), 7.31-7.21 (m, 1H), 7.10 (td, J=6.9, 6.5, 3.1 Hz, 1H), 5.13 (d, J=6.2 Hz, 1H), 4.73 (s, 1H), 3.94 (d, J=1.9 Hz, 3H), 1.68 (d, J=2.8 Hz, 3H), 0.88 (d, J=6.9 Hz, 1H)

### Step 5: rel-(2R,3S)-4-({[4,5-dichloro-3-(3,4-difluoro-2-methoxyphenyl)-3a-methyl-5a-(trifluoromethyl)hexahydrocyclobuta[2,1-b]furan-2-yl]carbonyl}amino)pyridin-2-carboxamide (59e)

DCE (500 mL), 59d (500 mg, 1.1 mmol, 1.0 eq), and 1,2-dichloroethylene (2 mL) were added sequentially in a quartz reaction flask to react under light at a wavelength of 275 nm for 3 weeks. The reaction system was separated and purified by column chromatography to recover 75 mg of raw material, and to obtain 20 mg of product with a yield of 3.4%, which was a white solid. MS: [M+H]⁺=554. ¹H NMR (400 MHz, Methanol-d₄) δ 8.53 (d, J=5.5 Hz, 1H), 8.31 (d, J=2.1 Hz, 1H), 7.93 (dd, J=5.5, 2.2 Hz, 1H), 7.12-7.05 (m, 2H), 5.41 (d, J=6.0 Hz, 1H), 4.69 (q, J=7.7 Hz, 2H), 4.61 (d, J=6.0 Hz, 1H), 4.01 (d, J=2.4 Hz, 3H), 0.95-0.93 (m, 3H).

### Step 6: rel-(2R,3S)-4-({[3-(3,4-difluoro-2-methoxyphenyl)-3a-methyl-5a-(trifluoromethyl)hexahydrocyclobuta[2,1-b]furan-2-yl]carbonyl}amino)pyridin-2-carboxamide (59)

Toluene (15 mL), 59e (10 mg, 0.02 mmol, 1.0 eq), tributyltin hydride (52 mg, 0.2 mmol, 10 eq), and AIBN (32 mg, 0.2 mmol, 10 eq) were added sequentially in a reaction flask and sealed to react at 100° C. for 36 h, until the completion of the reaction was monitored by LCMS. The reaction system was separated by thin-layer chromatography to obtain the compound 59 (3.4 mg, 40% yield), which was a white solid. MS: [M+H]⁺=486. ¹H NMR (400 MHz, Chloroform-d) δ 8.94 (s, 1H), 8.50 (d, J=5.4 Hz, 1H), 8.26 (s, 1H), 8.09 (s, 1H), 6.99-6.88 (m, 1H), 5.68 (s, 1H), 5.03 (s, 1H), 4.18 (s, 1H), 3.99 (d, J=2.3 Hz, 2H), 2.56 (dd, J=17.3, 6.7 Hz, 1H), 2.15 (t, J=13.1 Hz, 1H), 1.92 (dd, J=14.5, 6.3 Hz, 1H), 0.90 (s, 2H).

### Example 79: rel-(2R,3S,4S,5)-3-(3,4-difluoro-2-methoxyphenyl)-4,5-dimethyl-5-(trifluoromethyl)-N-(1,1,3-trioxo-2,3-dihydro-1λ6-benzo[d][1,2]thiazol-6-yl)tetrahydrofuran-2-carboxamide (79)

Example 79 was obtained by a method similar to that in Example 1 with 6-amino-2,3-dihydro-1λ6-benzo[d][1,2]thiazol-1,1,3-trione that replaced 1b in Example 1. ESI-MS m/z calc. 534.5, found 533.5 (M−1)⁻. ¹H NMR (400 MHz, DMSO-d₆) δ 10.59 (s, 1H), 7.97 (d, J=1.8 Hz, 1H), 7.72 (dd, J=8.2, 1.8 Hz, 1H), 7.52 (d, J=8.2 Hz, 1H), 7.17 (dd, J=8.5, 5.2 Hz, 2H), 5.10 (d, J=10.2 Hz, 1H), 4.26 (dd, J=10.2, 7.6 Hz, 1H), 3.95 (d, J=2.1 Hz, 3H), 2.77 (p, J=7.5 Hz, 1H), 1.61 (s, 3H), 0.76-0.60 (m, 3H).

### Example 80

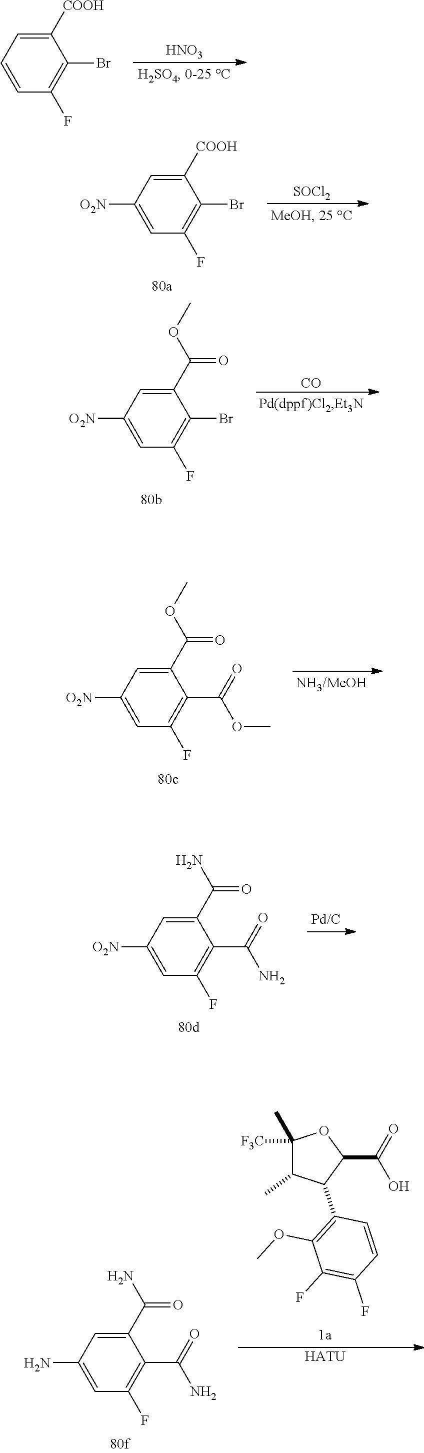

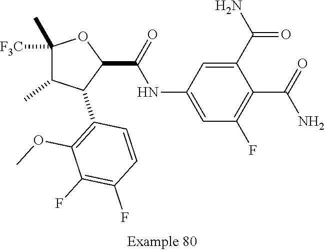

### Step 1: 2-bromo-3-fluoro-5-nitrobenzoic acid (80a)

The compound 2-bromo-3-fluorobenzoic acid (5 g, 22.8 mmol, 1.0 eq) was dissolved in concentrated sulfuric acid (15 mL), and fuming nitric acid (1 mL) was added at 0° C. The reaction mixture was stirred at 25° C. to react for 4 h, until the completion of the reaction was monitored by LCMS. The reaction solution was poured slowly into ice water and subjected to extraction with dichloromethane (50 mL×3). The organic phase was collected. The organic phase was washed with saturated saline (50 mL×3), dried over anhydrous sodium sulfate, filtered, and concentrated, to obtain a residue. The residue was purified by C18 column chromatography (0.1% FA/acetonitrile=1/4) to obtain the compound 2-bromo-3-fluoro-5-nitrobenzoic acid (800 mg, 13%), which was a yellow solid. LCMS: [M+H]⁺=263.9.

### Step 2: methyl 2-bromo-3-fluoro-5-nitrobenzoate (80b)

The compound 2-bromo-3-fluoro-5-nitrobenzoic acid 80a (800 mg, 3.03 mmol, 1.0 eq) was dissolved in methanol (5 mL), and thionyl chloride (1 mL) was added. The reaction mixture was stirred at 80° C. to react for 6 h, until the completion of the reaction was monitored by LCMS. The reaction solution was filtered to obtain a filtrate. The filtrate was concentrated to obtain the compound methyl 2-bromo-3-fluoro-5-nitrobenzoate 80b (800 mg, 95%), which was a yellow solid. LCMS: [M+H]⁺=277.9.

### Step 3: dimethyl 3-fluoro-5-nitrobenzoate (80c)

The compound methyl 2-bromo-3-fluoro-5-nitrobenzoate 80b (400 mg, 1.43 mmol, 1.0 eq) was dissolved in methanol (10 mL), and Pd(dppf)Cl₂ (32 mg, 0.143 mmol, 0.1 eq) and Et₃N (437 mg, 4.32 mmol, 3.0 eq) were added. The reaction solution was stirred in the presence of carbon monoxide (0.1 MPa) at 80° C. for 16 h. The reaction solution was concentrated to obtain a residue. The residue was purified by silica gel column chromatography (petroleum ether/ethyl acetate=10/1) to obtain the compound dimethyl 3-fluoro-5-nitrobenzoate 80c (40 mg, 11%), which was a brown oily substance. LCMS: [M+H]⁺=258.1.

### Step 4: Ammonium 3-fluoro-5-nitrophthalate (80d)

The compound dimethyl 3-fluoro-5-nitrobenzoate 80c (40 mg, 0.155 mmol, 1.0 eq) was dissolved in ammonia in methanol (5 mL). The reaction solution was stirred at 25° C. for 16 h. The reaction solution was concentrated to obtain a residue. The residue was washed with dichloromethane, and filtered. The solid was collected to obtain the compound ammonium 3-fluoro-5-nitrophthalate 80d (20 mg, 57%), which was a white solid. LCMS: [M+H]⁺=228.1.

### Step 5: ammonium 3-fluoro-5-aminophthalate (80e)

The compound ammonium 3-fluoro-5-nitrophthalate 80d (20 mg, 0.088 mmol, 1.0 eq) was dissolved in methanol (5 mL). The reaction solution was stirred in the presence of hydrogen at 25° C. for 1 h. The reaction solution was filtered, and the filtrate was collected and concentrated to obtain the compound ammonium 3-fluoro-5-aminophthalate 80e (13 mg, 75%), which was a yellow solid. LCMS: [M+H]⁺=198.1.

### Step 6: rel-5-((2R,3S,4S,5R)-3-(3,4-difluoro-2-methoxyphenyl)-4,5-dimethyl-5-(trifluoromethyl)tetrahydrofuran-2-carboxamide)-3-fluorophthalic amine (Example 80)

The compound 1a (28 mg, 0.079 mmol, 1.2 eq) was dissolved in DMF (3 mL), and the compound ammonium 3-fluoro-5-aminophthalate 80e (13 mg, 0.066 mmol, 1.0 eq), HATU (30 mg, 0.079 mmol, 1.2 eq), and DIEA (10 mg, 0.079 mmol, 1.2 eq) were added. The reaction solution was stirred at 25° C. for 1 h, until the completion of the reaction was monitored by LCMS. The reaction solution was purified by pre-HPLC (0.1% FA) to obtain the compound Example 80 (2.9 mg, 5%), which was a white solid. LCMS: [M+H]⁺=534.2. ¹H NMR (400 MHz, CD₃OD) δ 7.72 (dd, J=2.0, 2.0 Hz, 1H), 7.63-7.61 (m, 1H), 7.15-7.08 (m, 1H), 7.01-6.94 (m, 1H), 5.06 (d, J=10.4 Hz, 1H), 4.33-4.28 (m, 1H), 3.99 (d, J=2.4 Hz, 3H), 2.85-2.71 (m, 1H), 1.65 (s, 3H), 0.83-0.80 (m, 3H).

### Example 81

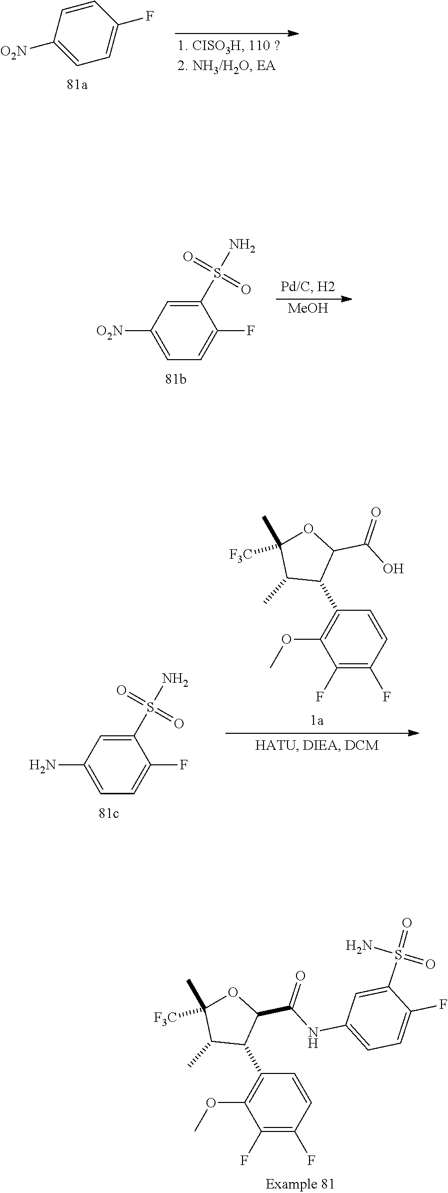

### Step 1: 2-fluoro-5-nitrobenzenesulfonamide (81b)

The compound p-fluoronitrobenzene 81a (2.0 g, 14.2 mmol, 1.0 eq) was dissolved in chlorosulfonic acid (10 mL). The reaction mixture was stirred at 110° C. to react for 24 h. The reaction solution was cooled down to room temperature. The reaction solution was poured slowly into an ice bath, and subjected to extraction with ethyl acetate (50 mL×3) when all the ice melted. The organic phase was collected. The organic phase was washed with saturated saline (50 mL×3), dried over anhydrous sodium sulfate, filtered, and concentrated, to obtain a residue. The residue was dissolved in ethyl acetate (60 mL), and aqueous ammonia (60 mL) was added slowly at 0° C. The reaction mixture was stirred at 25° C. for 16 h. After the completion of the reaction was monitored by TLC, the reaction solution was poured into water and subjected to extraction with ethyl acetate (50 mL×3). The organic phase was collected. The organic phase was washed with saturated saline (50 mL×3), dried over anhydrous sodium sulfate, filtered, and concentrated, to obtain a residue. The residue was purified by silica gel column chromatography (petroleum ether/ethyl acetate=2/3) to obtain the compound 2-fluoro-5-nitrobenzenesulfonamide 81b (400 mg, 13% yield), which was a yellow solid. ¹H NMR (400 MHz, DMSO) δ 8.56-8.53 (m, 2H), 8.06 (s, 2H), 7.86-7.65 (m, 1H).

### Step 2: 5-amino-2-fluorobenzenesulfonamide (81c)

The compound 2-fluoro-5-nitrobenzenesulfonamide 81b (200 mg, 0.90 mmol, 1.0 eq) was dissolved in methanol (5 mL), and wet palladium on carbon (193 mg, 1.80 mmol, 2.0 eq) was added. The reaction mixture was stirred in the presence of hydrogen at 25° C. to react for 1 h, until the completion of the reaction was monitored by LCMS. The reaction solution was filtered to obtain a filtrate. The filtrate was concentrated to obtain the compound 5-amino-2-fluorobenzenesulfonamide (160 mg, 92% yield), which was a black solid. LCMS: [M+H]⁺=191.1

### Step 3: rel-(2R,3S,4S,5R)-3-(3,4-difluoro-2-methoxyphenyl)-N-(4-fluoro-3-sulfonatophenyl)-4,5-dimethyl-5-(trifluoromethyl)tetrahydrofuran-2-carboxamide (Example 81)

The compound (3S,4S,5R)-3-(3,4-difluoro-2-methoxyphenyl)-4,5-dimethyl-5-(trifluoromethyl)tetrahydrofuran-2-carboxylic acid 1a (50 mg, 0.14 mmol, 1.0 eq) was dissolved in dichloromethane (3 mL), and the compound 5-amino-2-fluorobenzenesulfonamide 81c (32 mg, 0.17 mmol, 1.2 eq), benzotriazole-N,N,N′,N′-tetramethyluronium hexafluorophosphate (64 mg, 0.17 mmol, 1.2 eq), and N,N-diisopropylethylamine (22 mg, 0.17 mmol, 1.2 eq) were added. The reaction solution was stirred at 25° C. for 1 h. The reaction solution was concentrated to obtain a residue. The residue was purified by preparative separation (0.1% formic acid) to obtain the compound (2R,3S,4S,5R)-3-(3,4-difluoro-2-methoxyphenyl)-N-(4-fluoro-3-sulfonatophenyl)-4,5-dimethyl-5-(trifluoromethyl)tetrahydrofuran-2-carboxamide (2.3 mg, 3% yield), which was a white solid. LCMS: [M+H]⁺=527.1. ¹H NMR (400 MHz, DMSO) δ 10.61 (s, 1H), 8.57 (s, 1H), 8.23-8.21 (m, 1H), 7.93-7.86 (m, 1H), 7.72 (s, 1H), 7.46-7.39 (m, 1H), 7.29-7.18 (m, 2H), 5.13 (d, J=10.4 Hz, 1H), 4.35-4.25 (m, 1H), 4.01 (d, J=2.4 Hz, 3H), 2.85-2.79 (m, 1H), 1.66 (s, 3H), 0.79 (d, J=5.6 Hz, 3H).

### Example 82

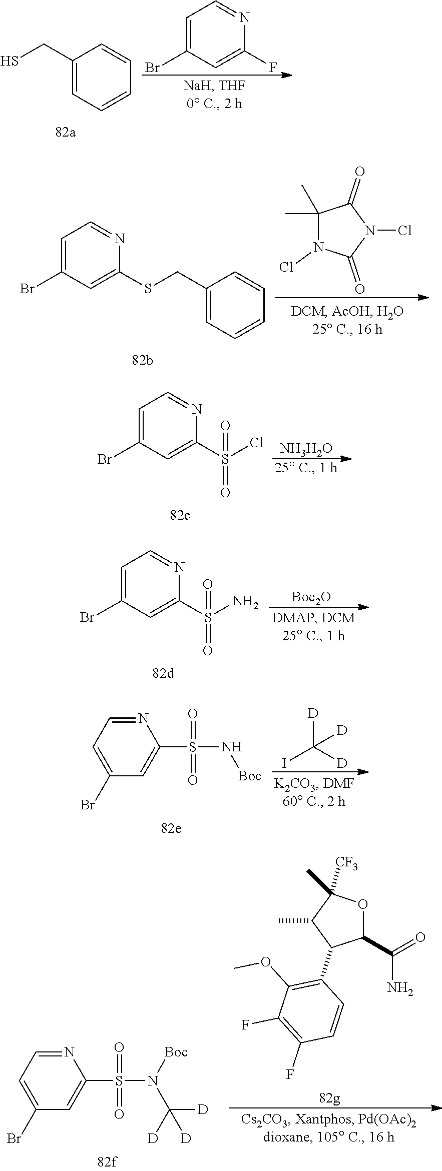

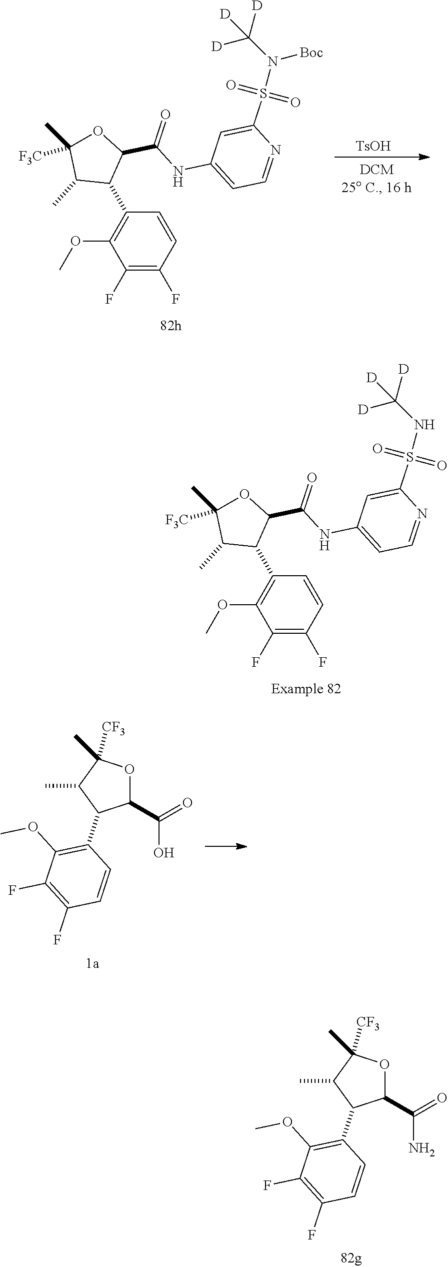

### Step 1: 2-(benzylthio)-4-bromopyridine (82b)

The compound benzyl mercaptan 82a (2.0 g, 16.1 mmol, 1.0 eq) was dissolved in anhydrous tetrahydrofuran (30 mL), and sodium hydride (640 mg, 16.1 mmol, 1.0 eq) was added at 0° C. under nitrogen protection. The reaction solution was stirred at 0° C. for 30 min. A solution of the compound 4-bromo-2-fluoropyridine (2.8 g, 16.1 mmol, 1.0 eq) in anhydrous tetrahydrofuran (10 mL) was added dropwise at this temperature, and then stirred at 0° C. for 1.5 h. After the completion of the reaction of the raw material was monitored by LCMS, the reaction solution with water (50 mL) added was subjected to extraction with ethyl acetate (20 mL×3), washed with saturated saline, dried over anhydrous sodium sulfate, and then concentrated. The residue was purified by silica gel column chromatography (petroleum ether/ethyl acetate=50/1) to obtain the compound 2-(benzylthio)-4-bromopyridine 82b (4.3 g, 95% yield), which was a pink oily substance. LCMS: [M+H]⁺=280.0

### Step 2: 4-bromopyridine-2-sulfonyl chloride (82c)

The compound 2-(benzylthio)-4-bromopyridine 82b (4.3 g, 15.3 mmol, 1.0 eq) was dissolved in dichloromethane, acetic acid, and water in 7:1:2 (80 mL), and 1,3-dichloro-5,5-dimethylimidazolidine-2,4-dione (1.1 g, 7.2 mmol, 0.7 eq) was added. The reaction solution reacted at 25° C. under nitrogen protection for 16 h. After the completion of the reaction of the raw material was monitored by TLC, the reaction solution with water (20 mL) added was subjected to extraction with dichloromethane (20 mL×3), washed with saturated saline, dried over anhydrous sodium sulfate, concentrated, diluted with dichloromethane, and filtered. The filtrate was spin-dried to obtain the compound 4-bromopyridine-2-sulfonyl chloride (4.5 g, 92% yield), which was a yellow oily substance.

### Step 3: 4-bromopyridine-2-sulfonamide (82d)

Aqueous ammonia (10 mL) was added to the compound 4-bromopyridine-2-sulfonyl chloride 82c (3.0 g, 9.4 mmol, 1.0 eq). The reaction solution reacted at 25° C. for 1 h. After the completion of the reaction of the raw material was monitored by LCMS, the reaction solution was concentrated. The residue was purified by silica gel column chromatography (petroleum ether/ethyl acetate=1/1) to obtain the compound 4-bromopyridine-2-sulfonamide 82d (1.7 g, 77% yield), which was a gray solid. LCMS: [M+H]⁺=238.9

### Step 4: ((4-bromopyridin-2-yl)sulfonyl)tert-butyl carbamate (82e)

The compound 4-bromopyridine-2-sulfonamide 82d (500 mg, 2.1 mmol, 1.0 eq) was dissolved in dichloromethane (10 mL), and triethylamine (213 mg, 2.1 mmol, 1.0 eq), 4-dimethylaminopyridine (13 mg, 0.10 mmol, 0.05 eq), and di-tert-butyl dicarbonate (483 mg, 2.2 mmol, 1.05 eq) were added. The reaction solution reacted at 25° C. for 1 h. After the completion of the reaction of the raw material was monitored by LCMS, the reaction solution was concentrated. The residue was purified by silica gel column chromatography (dichloromethane/methanol=10/1) to obtain the compound ((4-bromopyridin-2-yl)sulfonyl)tert-butyl carbamate 82e (611 mg, 85% yield), which was a yellow oily substance. LCMS: [M+Na]⁺=359.0

### Step 5: ((4-bromopyridin-2-yl)sulfonyl)(methyl-d3)tert-butyl carbamate (82f)

The compound ((4-bromopyridin-2-yl)sulfonyl)tert-butyl carbamate 82e (200 mg, 0.59 mmol, 1.0 eq) was dissolved in anhydrous N,N-dimethylformamide (5 mL), and potassium carbonate (123 mg, 0.88 mmol, 1.5 eq) and deuterated iodomethane (94 mg, 0.65 mmol, 1.1 eq) were added. The reaction solution reacted at 60° C. for 2 h. After the completion of the reaction of the raw material was monitored by LCMS, the reaction solution with water (10 mL) added was subjected to extraction with ethyl acetate (10 mL×3), washed with water and saturated saline sequentially, dried over anhydrous sodium sulfate, and then concentrated. The residue was purified by silica gel column chromatography (petroleum ether/ethyl acetate=10/1) to obtain 82f (130 mg, 62% yield), which was a white solid. LCMS: [M+Na]⁺=376.0

### Step 6: rel-(2R,3S,4S,5R)-3-(3,4-difluoro-2-methoxyphenyl)-4,5-dimethyl-5-(trifluoromethyl)tetrahydrofuran-2-carboxamide (82g)

The intermediate 82g was obtained by a method similar to that in Example 1 with ammonium chloride that replaced 1b in Example 1. LCMS: [M+H]⁺=354.3.

### Step 7: rel-((4-((2R,3S,4S,5R)-3-(3,4-difluoro-2-methoxyphenyl)-4,5-dimethyl-5-(trifluoromethyl)tetrahydrofuran-2-carboxamido)pyridin-2-yl)sulfonyl)(methyl-d3)tert-butyl carbamate (82h)

The compound 82f (70 mg, 0.20 mmol, 1.0 eq) was dissolved in dioxane (5 mL), and the compound 82g (139 mg, 0.24 mmol, 1.2 eq), 4,5-bis(diphenylphosphino)-9,9-dimethylxanthene (23 mg, 0.04 mmol, 0.1 eq), cesium carbonate (128 mg, 0.39 mmol, 2.0 eq), and palladium acetate (3 mg, 0.02 mmol, 0.1 eq) were added. The reaction solution reacted at 105° C. under nitrogen protection for 16 h. After the completion of the reaction of the raw material was monitored by LCMS, the reaction solution was concentrated. The residue was purified by silica gel column chromatography (petroleum ether/ethyl acetate=5/1) to obtain 82h (65 mg, 52% yield), which was a yellow oily substance. LCMS: [M+H]⁺=627.2

### Step 8: rel-(2R,3S,4S,5R)-3-(3,4-difluoro-2-methoxyphenyl)-4,5-dimethyl-N-(2-(N-(methyl-d3)sulfamoyl)pyridin-4-yl)-5-(trifluoromethyl)tetrahydrofuran-2-carboxamide (82h)

Step 8: rel-(2R,3S,4S,5R)-3-(3,4-difluoro-2-methoxyphenyl)-4,5-dimethyl-N-(2-(N-(methyl-d3)sulfamoyl)pyridin-4-yl)-5-(trifluoromethyl)tetrahydrofuran-2-carboxamide

82h (50 mg, 0.08 mmol, 1.0 eq) was dissolved in dichloromethane (5 mL), and p-toluenesulfonic acid (13.72 mg, 0.08 mmol, 1.0 eq) was added. The reaction solution reacted at 25° C. for 16 h. After the completion of the reaction of the raw material was monitored by LCMS, the reaction mixture was concentrated. The residue was purified by preparative separation (0.05% NH₃) to obtain Example 82 (10.0 mg, 24% yield), which was an off-white solid. LCMS: [M+H]⁺=527.1 ¹H NMR (400 MHz, CH₃OD) δ 8.54 (d, J=5.6 Hz, 1H), 8.30 (d, J=2.0 Hz, 1H), 7.86 (dd, J=5.6, 2.0 Hz, 1H), 7.14-7.09 (m, 1H), 7.02-6.94 (m, 1H), 5.08 (d, J=10.4 Hz, 1H), 4.32 (dd, J=10.4, 8.4 Hz, 1H), 3.99 (d, J=2.4 Hz, 3H), 2.80 (p, J=7.6 Hz, 1H), 1.65 (s, 3H), 0.83-0.79 (m, 3H).

### Example 83

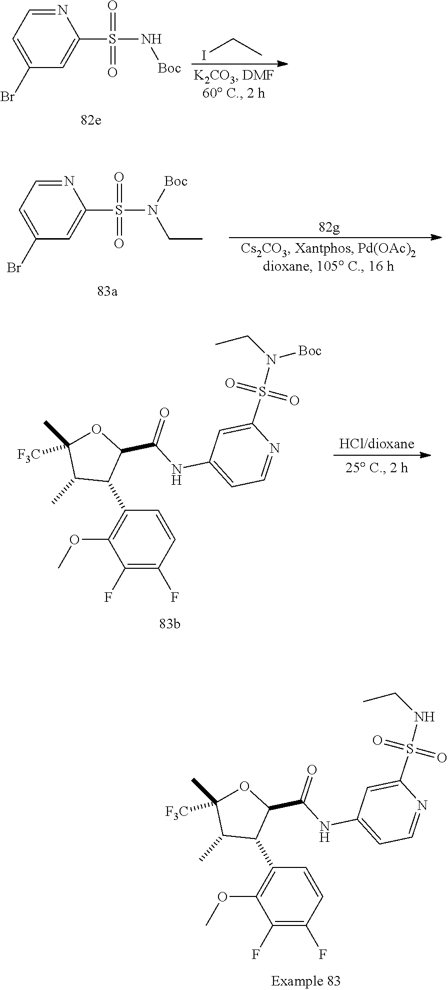

### Step 1: ((4-bromopyridin-2-yl)sulfonyl)(ethyl)tert-butyl carbamate (83a)

The compound ((4-bromopyridin-2-yl)sulfonyl)tert-butyl carbamate 82e (200 mg, 0.59 mmol, 1.0 eq) was dissolved in anhydrous N,N-dimethylformamide (5 mL), and potassium carbonate (123 mg, 0.88 mmol, 1.5 eq) and iodoethane (101 mg, 0.65 mmol, 1.1 eq) were added. The reaction solution reacted at 60° C. for 2 h. After the completion of the reaction of the raw material was monitored by LCMS, the reaction solution with water (10 mL) added was subjected to extraction with ethyl acetate (10 mL×3), washed with water and saturated saline sequentially, dried over anhydrous sodium sulfate, and then concentrated. The residue was purified by silica gel column chromatography (petroleum ether/ethyl acetate=10/1) to obtain the compound ((4-bromopyridin-2-yl)sulfonyl)(ethyl)tert-butyl carbamate 83a (130 mg, 60% yield), which was a yellow oily substance. LCMS: [M+Na]⁺=387.0

### Step 2: rel-((4-((2R,3S,4S,5R)-3-(3,4-difluoro-2-methoxyphenyl)-4,5-dimethyl-5-(trifluoromethyl)tetrahydrofuran-2-carboxamido)pyridin-2-yl)sulfonyl)(ethyl)tert-butyl carbamate (83b)

The compound 83a (130 mg, 0.35 mmol, 1.0 eq) was dissolved in dioxane (5 mL), and 82g (197 mg, 0.39 mmol, 1.2 eq), 4,5-bis(diphenylphosphino)-9,9-dimethylxanthene (41 mg, 0.07 mmol, 0.2 eq), cesium carbonate (231 mg, 0.71 mmol, 2.0 eq), and palladium acetate (6 mg, 0.03 mmol, 0.1 eq) were added. The reaction solution reacted at 105° C. under nitrogen protection for 16 h. After the completion of the reaction of the raw material was monitored by LCMS, the reaction solution was concentrated. The residue was purified by silica gel column chromatography (petroleum ether/ethyl acetate=4/1) to obtain 83b (100 mg, 44% yield), which was a yellow oily substance. LCMS: [M+H]⁺=638.2

### Step 3: rel-(2R,3S,4S,5R)-3-(3,4-difluoro-2-methoxyphenyl)-N-(2-(N-ethylsulfamoyl)pyridin-4-yl)-4,5-dimethyl-5-(trifluoromethyl)tetrahydrofuran-2-carboxamide (Example 83)

The compound 83b (100 mg, 0.16 mmol, 1.0 eq) was dissolved in hydrochloric acid/dioxane (4 M, 30 mL). The reaction solution reacted at 25° C. for 2 h. After the completion of the reaction of the raw material was monitored by LCMS, the reaction mixture was concentrated. The residue was purified by preparative separation (0.01% FA) to obtain Example 83 (39.5 mg, 47% yield), which was a white solid. LCMS: [M+H]⁺=538.1

¹H NMR (400 MHz, DMSO) δ 10.88 (s, 1H), 8.59 (d, J=5.2 Hz, 1H), 8.27 (d, J=1.6 Hz, 1H), 7.91-7.75 (m, 2H), 7.18-7.15 (m, 2H), 5.13 (d, J=10.0 Hz, 1H), 4.28-4.23 (m, 1H), 3.95 (d, J=2.4 Hz, 3H), 2.97-2.86 (m, 2H), 2.80-2.75 (m, 1H), 1.61 (s, 3H), 0.97 (t, J=7.2 Hz, 3H), 0.73 (d, J=6.0 Hz, 3H).

### Example 84

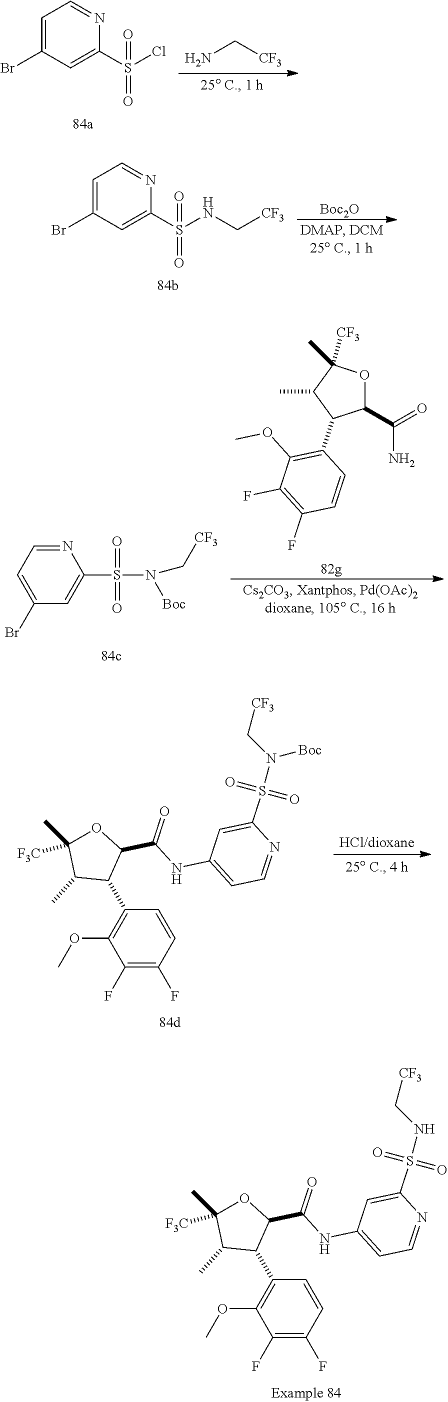

### Step 1: 4-bromo-N-(2,2,2-trifluoroethyl)pyridin-2-sulfonamide (84b)

The compound 2,2,2-trifluoroethan-1-amine (1.2 g, 11.7 mmol, 3.0 eq) was added to the compound 4-bromopyridine-2-sulfonyl chloride 84a (1.0 g, 3.9 mmol, 1.0 eq). The reaction solution reacted at 25° C. for 1 h. After the completion of the reaction of the raw material was monitored by LCMS, the reaction solution was concentrated. The residue was purified by silica gel column chromatography (petroleum ether/ethyl acetate=10/1) to obtain the compound 4-bromo-N-(2,2,2-trifluoroethyl)pyridin-2-sulfonamide 84b (700 mg, 56% yield), which was a white solid. LCMS: [M+H]⁺=318.9

### Step 2: ((4-bromopyridin-2-yl)sulfonyl)(2,2,2-trifluoroethyl)tert-butyl carbamate (84c)

The compound 4-bromo-N-(2,2,2-trifluoroethyl)pyridin-2-sulfonamide 84b (700 mg, 2.2 mmol, 1.0 eq) was dissolved in dichloromethane (10 mL), and potassium carbonate (604 mg, 4.4 mmol, 2.0 eq), 4-dimethylaminopyridine (27 mg, 0.22 mmol, 0.1 eq), and di-tert-butyl dicarbonate (573 mg, 2.6 mmol, 1.2 eq) were added. The reaction solution reacted at 25° C. for 1 h. After the completion of the reaction of the raw material was monitored by LCMS, the reaction solution was concentrated. The residue was purified by silica gel column chromatography (petroleum ether/ethyl acetate=10/1) to obtain the compound ((4-bromopyridin-2-yl)sulfonyl)(2,2,2-trifluoroethyl)tert-butyl carbamate 84c (700 mg, 76% yield), which was a yellow oily substance. LCMS: [M+Na]⁺=441.0

### Step 3: rel-((4-((2R,3S,4S,5R)-3-(3,4-difluoro-2-methoxyphenyl)-4,5-dimethyl-5-(trifluoromethyl)tetrahydrofuran-2-carboxamido)pyridin-2-yl)sulfonyl)(2,2,2-trifluoroethyl)tert-butyl carbamate (84d)

The compound 84c (100 mg, 0.24 mmol, 1.0 eq) was dissolved in dioxane (5 mL), and the compound 82g (154 mg, 0.26 mmol, 1.1 eq), 4,5-bis(diphenylphosphino)-9,9-dimethylxanthene (27 mg, 0.048 mmol, 0.2 eq), cesium carbonate (155 mg, 0.48 mmol, 2.0 eq), and palladium acetate (4 mg, 0.024 mmol, 0.1 eq) were added. The reaction solution reacted at 105° C. under nitrogen protection for 16 h. After the completion of the reaction of the raw material was monitored by LCMS, the reaction solution was concentrated. The residue was purified by silica gel column chromatography (dichloromethane/methanol=10/1) to obtain the compound 84d (80 mg, 48% yield), which was a yellow oily substance. LCMS: [M+H]⁺=692.1

### Step 4: rel-(2R,3S,4S,5R)-3-(3,4-difluoro-2-methoxyphenyl)-4,5-dimethyl-N-(2-(N-(2,2,2-trifluoroethyl)sulfamoyl)pyridin-4-yl)-5-trifluoromethyl tetrahydrofuran-2-carboxamide (Example 84)

The compound 84d (80 mg, 0.11 mmol, 1.0 eq) was dissolved in hydrochloric acid/dioxane (4 M, 5 mL). The reaction solution reacted at 25° C. for 4 h. After the completion of the reaction of the raw material was monitored by LCMS, the reaction mixture was concentrated. The residue was purified by preparative separation (0.1% FA) to obtain Example 84 (15.4 mg, 22% yield), which was a white solid. LCMS: [M+H]⁺=592.1. ¹H NMR (400 MHz, DMSO-d₆) δ 10.88 (s, 1H), 8.84 (t, J=6.8 Hz, 1H), 8.59 (d, J=5.2 Hz, 1H), 8.29 (d, J=2.0 Hz, 1H), 7.87-7.84 (m, 1H), 7.20-7.14 (m, 2H), 5.13 (d, J=10.4 Hz, 1H), 4.28-4.23 (m, 1H), 3.95 (d, J=2.24 Hz, 3H), 3.84-3.75 (m, 2H), 2.82-2.72 (m, 1H), 1.61 (s, 3H), 0.73 (d, J=6.0 Hz, 3H).

### Example 85: Synthesis of Compound 85

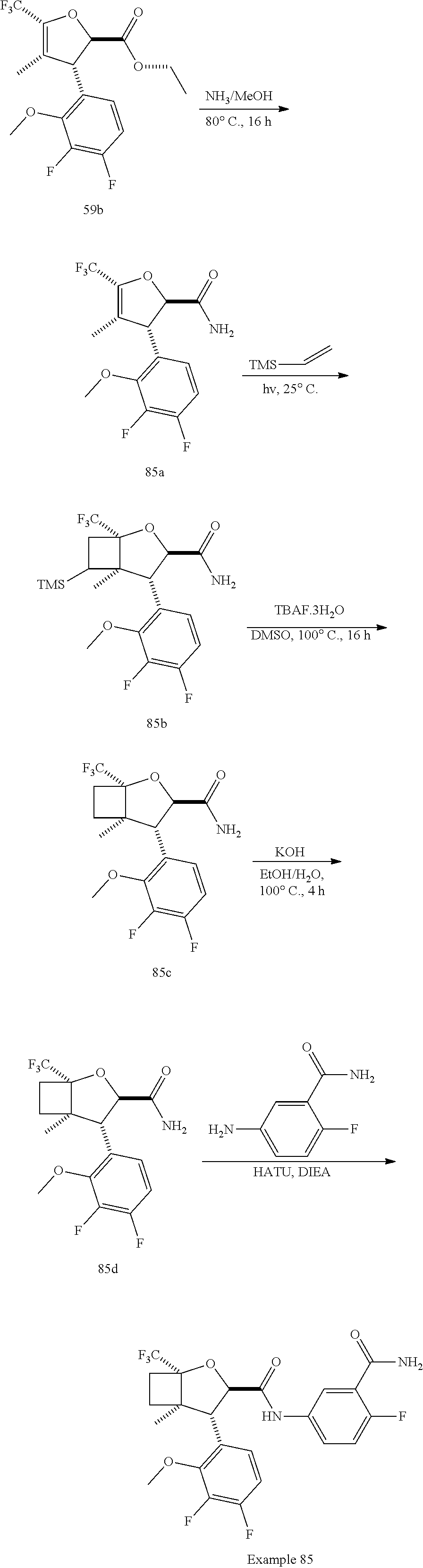

### Step 1: rel-(2R,3S)-3-(3,4-difluoro-2-methoxyphenyl)-4-methyl-5-(trifluoromethyl)-2,3-dihydrofuran-2-carboxamide (85a)

The compound 59b (5 g, 13.6 mmol, 1.0 eq) was dissolved in ammonia/methanol (7 M, 15 mL). The reaction solution reacted at 80° C. for 16 h. After the completion of the reaction of the raw material was monitored by LCMS, the reaction solution was concentrated. The residue was purified by silica gel column chromatography (dichloromethane/methanol=20/1) to obtain the compound 85a (3.8 g, 83% yield), which was a white solid. LCMS: [M+H]⁺=338.0

### Step 2: rel-(1R,3R,4R,5R)-4-(3,4-difluoro-2-methoxyphenyl)-5-methyl-1-(trifluoromethyl)-6-(trimethylsilyl)-2-oxabicyclo[3.2.0]heptane-3-carboxamide (85b)

The compound 85a (2.0 g, 5.9 mmol, 1.0 eq) was dissolved in dichloroethane (50 mL), and the compound trimethyl(vinyl)silane (11.8 g, 11.8 mmol, 20.0 eq) was added. The reaction solution was irradiated under a mercury lamp to react at 25° C. for 16 h. The reaction mixture was concentrated. The residue was purified by preparative separation (0.01% FA/ACN=1/1) to obtain the compound 85b (600 mg, 23% yield), which was a yellow oily substance. LCMS: [M+H]⁺=438.2

### Step 3: rel-(1R,3R,4S,5R)-4-(3,4-difluoro-2-methoxyphenyl)-5-methyl-1-trifluoromethyl-2-oxabicyclo[3.2.0]heptane-3-carboxamide (85c)

The compound 85b (500 mg, 3.0 mmol, 1.0 eq) was dissolved in dimethyl sulfoxide (20 mL), and tetrabutylammonium fluoride trihydrate (6.4 g, 22.8 mmol, 20 eq) was added. The reaction solution reacted at 100° C. for 16 h. The reaction mixture was concentrated. The residue was purified by preparative separation (0.01% FA/ACN=3/2) to obtain the compound 85c (170 mg, 40% yield), which was a yellow oily substance. LCMS: [M+H]⁺=366.1

### Step 4: rel-(1R,3R,4S,5R)-4-(3,4-difluoro-2-methoxyphenyl)-5-methyl-1-trifluoromethyl-2-oxabicyclo[3.2.0]heptane-3-carboxylic acid (85d)

The compound 85c (170 mg, 0.387 mmol, 1.0 eq) was dissolved in ethanol/water (3 mL+1 mL), and potassium hydroxide (217 mg, 3.87 mmol, 10 eq) was added. The reaction solution reacted at 100° C. for 4 h, until the completion of the reaction was monitored by LCMS. The reaction solution was adjusted with 1 M dilute hydrochloric acid to pH 5-6, and subjected to extraction with ethyl acetate (10 mL×3). The organic phase was collected. The organic phase was washed with saturated saline, dried over anhydrous sodium sulfate, and then concentrated, to obtain the compound 85d (100 mg, 70% yield), which was a yellow oily substance. LCMS: [M+H]⁺=367.1

### Step 5: rel-(1R,3R,4S,5R)—N-(3-carbamoyl-4-fluorophenyl)-4-(3,4-difluoro-2-methoxyphenyl)-5-methyl-1-trifluoromethyl-2-oxabicyclo[3.2.0]heptane-3-carboxamide

### Example 85

The compound 85d (50 mg, 0.14 mmol, 1.0 eq) was dissolved in dichloromethane (10 mL), and the compound 5-amino-2-fluorobenzamide (25 mg, 0.16 mmol, 1.2 eq), benzotriazole-N,N,N′,N′-tetramethyluronium hexafluorophosphate (62 mg, 0.16 mmol, 1.2 eq), and N,N-diisopropylethylamine (21 mg, 0.16 mmol, 1.2 eq) were added. The reaction solution was stirred at 25° C. for 2 h, until the completion of the reaction was monitored by LCMS. The reaction solution was concentrated to obtain a residue. The residue was purified by preparative separation (0.01% FA) to obtain Example 85 (7.5 mg, 9% yield), which was a white solid. LCMS: [M+H]⁺=503.1. ¹H NMR (400 MHz, CD₃OD) δ 7.91-7.89 (m, 1H), 7.72-7.67 (m, 1H), 7.11-7.06 (m, 1H), 7.03-6.95 (m, 1H), 6.93-6.84 (m, 1H), 4.96 (d, J=7.6 Hz, 1H), 4.22 (d, J=7.6 Hz, 1H), 3.88 (d, J=2.0 Hz, 3H), 2.42-2.34 (m, 1H), 2.33-2.25 (m, 1H), 2.16-2.09 (m, 1H), 1.83-1.75 (m, 1H), 0.81 (d, J=1.2 Hz, 3H).

Example 85 was subjected to chiral resolution by SFC with a column: Daicel CHIRALPAK 1H_3, 3.0×150 mm, 3 μm particle size, to obtain Example 85-1 (RT=1.4 min) and Example 85-2 (RT=2.0 min).

### Example 86: rel-4-((1R,3R,4S,5R)-4-(3,4-difluoro-2-methoxyphenyl)-5-methyl-1-(trifluoromethyl)-2-oxabicyclo[3.2.0]heptane-3-formylamino)phthalamide (86)

Example 86 was prepared according to a method similar to step 5 in Example 85. LCMS: [M+H]⁺=528.1. ¹H NMR (400 MHz, MeOD) δ7.89 (s, 1H), 7.80 (d, J=8.4 Hz, 1H), 7.63 (d, J=8.4 Hz, 1H), 7.05 (dd, J=25.2, 18.0 Hz, 2H), 5.10 (d, J=8.0 Hz, 1H), 4.36 (d, J=8.0 Hz, 1H), 4.07-3.92 (m, 3H), 2.68 (s, 1H), 2.46-2.17 (m, 2H), 1.98-1.80 (m, 1H), 1.32 (s, 1H), 0.93 (s, 3H).

### Example 87

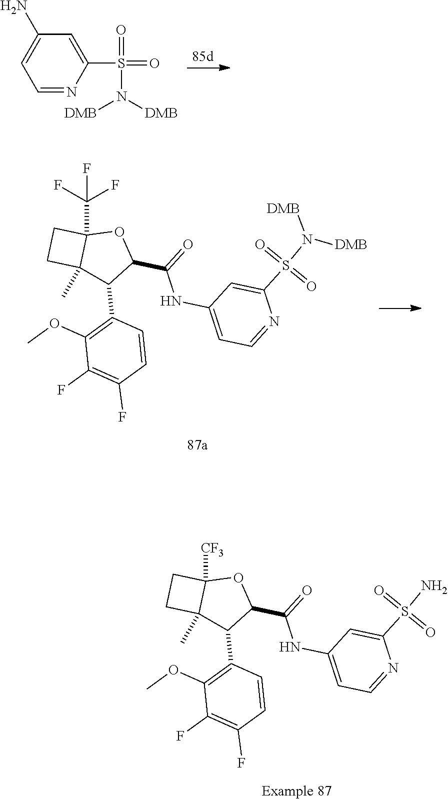

### Step 1: rel-(1R,3R,4S,5R)-2-(2-(3,4-dimethoxybenzyl)sulfamoyl)pyridin-4-yl)-4-(3,4-difluoro-2-methoxyphenyl)-5-methyl-1-trifluoromethyl-2-oxabicyclo[3.2.0]heptane-3-carboxamide (87a)

The compound 85d (60 mg, 0.16 mmol, 1.0 eq) was dissolved in anhydrous pyridine (3 mL), and the compound 4-amino-N,N-bis(3,4-dimethoxybenzyl)pyridin-2-sulfonamide 34e (78 mg, 0.16 mmol, 1.0 eq) and phosphorus oxychloride (100 mg, 0.66 mmol, 4.0 eq) were added. The reaction solution reacted at 0° C. for 1.5 h. After the completion of the reaction of the raw material was monitored by LCMS, the reaction mixture was diluted with water (10 mL), subjected to extraction with ethyl acetate (10 mL×3), washed with water and saturated saline sequentially, dried over anhydrous sodium sulfate, and then concentrated, to obtain the compound 87a (120 mg, 89% yield), which was a yellow oily substance.

### Step 2: rel-(1R,3R,4S,5R)-4-(3,4-difluoro-2-methoxyphenyl)-5-methyl-N-(2-sulfamoylpyridin-4-yl)-1-trifluoromethyl-2-oxabicyclo[3.2.0]heptane-3-carboxamide (Example 87)

The compound 87a (120 mg, 0.15 mmol, 1.0 eq) was dissolved in dichloromethane (5 mL), and trifluoroacetic acid (5 mL) was added. The reaction solution reacted at 25° C. for 4 h. After the completion of the reaction of the raw material was monitored by LCMS, the reaction mixture was concentrated. The residue was purified by preparative separation (0.05% FA) to obtain Example 87 (17.2 mg, 23% yield), which was a white solid. LCMS: [M+H]⁺=522.1. ¹H NMR (400 MHz, DMSO-d₆) δ 10.89 (s, 1H), 8.59 (d, J=5.6 Hz, 1H), 8.32 (d, J=2.0 Hz, 1H), 7.90-7.88 (m, 1H), 7.44 (s, 2H), 7.23-7.15 (m, 1H), 7.12-7.07 (m, 1H), 5.16 (d, J=8.0 Hz, 1H), 4.23 (d, J=8.0 Hz, 1H), 3.93 (d, J=2.0 Hz, 3H), 2.44-2.35 (m, 2H), 2.18-2.10 (m, 1H), 1.87-1.80 (m, 1H), 0.82 (s, 3H).

### Example 88: Synthesis of Compound 88

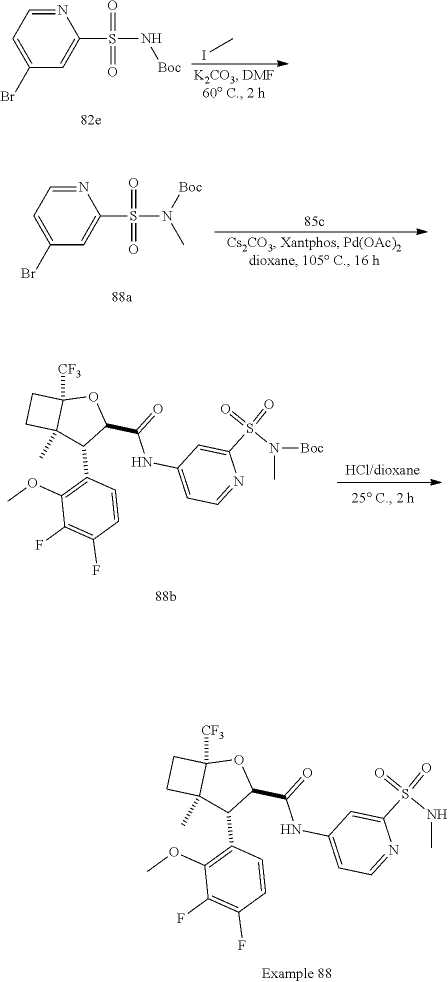

### Step 1: compound ((4-bromopyridin-2-yl)sulfonyl)(methyl)tert-butyl carbamate (88a)

The compound ((4-bromopyridin-2-yl)sulfonyl)tert-butyl carbamate 82e (100 mg, 0.30 mmol, 1.0 eq) was dissolved in anhydrous tetrahydrofuran (5 mL), and potassium carbonate (82 mg, 0.59 mmol, 2.0 eq) and iodomethane (50 mg, 0.35 mmol, 1.2 eq) were added. The reaction solution reacted at 60° C. for 2 h. After the completion of the reaction of the raw material was monitored by LCMS, the reaction solution with water (10 mL) added was subjected to extraction with ethyl acetate (10 mL×3), washed with water and saturated saline sequentially, dried over anhydrous sodium sulfate, and then concentrated. The residue was purified by silica gel column chromatography (petroleum ether/ethyl acetate=16/1) to obtain 88a (100 mg, 96% yield), which was a white solid. LCMS: [M+Na]⁺=373.0

### Step 2: rel-((4-((1R,3R,4S,5R)-4-(3,4-difluoro-2-methoxyphenyl)-5-methyl-1-(trifluoromethyl)-2-oxabicyclo[3.2.0]heptane-3-carboxamido)pyridin-2-yl)sulfonyl)(methyl)tert-butyl carbamate (88b)

The compound 88a (53 mg, 0.15 mmol, 1.1 eq) was dissolved in dioxane (5 mL), and the compound 85c (50 mg, 0.14 mmol, 1.0 eq), 4,5-bis(diphenylphosphino)-9,9-dimethylxanthene (15 mg, 0.027 mmol, 0.2 eq), cesium carbonate (89 mg, 0.27 mmol, 2.0 eq), and palladium acetate (2 mg, 0.013 mmol, 0.1 eq) were added. The reaction solution reacted at 105° C. under nitrogen protection for 16 h. After the completion of the reaction of the raw material was monitored by LCMS, the reaction solution was concentrated. The residue was purified by silica gel column chromatography (dichloromethane/methanol=10/1) to obtain the compound 88b (70 mg, 80% yield), which was a yellow oily substance. LCMS: [M+H]⁺=636.2

### Step 3: rel-(1R,3R,4S,5R)-4-(3,4-difluoro-2-methoxyphenyl)-5-methyl-N-(2-(N-methylsulfamoyl)pyridin-4-yl)-1-(trifluoromethyl)-2-oxabicyclo[3.2.0]heptane-3-carboxamide (Example 88)

The compound 88b (70 mg, 0.11 mmol, 1.0 eq) was dissolved in hydrochloric acid/dioxane (4 M, 10 mL). The reaction solution reacted at 25° C. for 4 h. After the completion of the reaction of the raw material was monitored by LCMS, the reaction mixture was concentrated. The residue was purified by preparative separation (0.01% FA) to obtain Example 88 (4.2 mg, 7.1% yield), which was a white solid. LCMS: [M+H]⁺=536.1.

¹H NMR (400 MHz, CD₃OD) δ 8.54 (d, J=5.6 Hz, 1H), 8.25 (d, J=2.0 Hz, 1H), 7.81 (dd, J=2.4, 2.0 Hz, 1H), 7.32-7.26 (m, 1H), 7.06-6.95 (m, 1H), 5.24 (d, J=10.4 Hz, 1H), 3.92 (d, J=1.6 Hz, 3H), 3.84 (d, J=10.8 Hz, 1H), 2.63 (s, 3H), 2.58-2.51 (m, 1H), 2.48-2.39 (m, 1H), 2.15-2.08 (m, 1H), 1.49-1.48 (m, 1H), 1.29 (s, 3H).

### Example 89: Synthesis of Compound 89

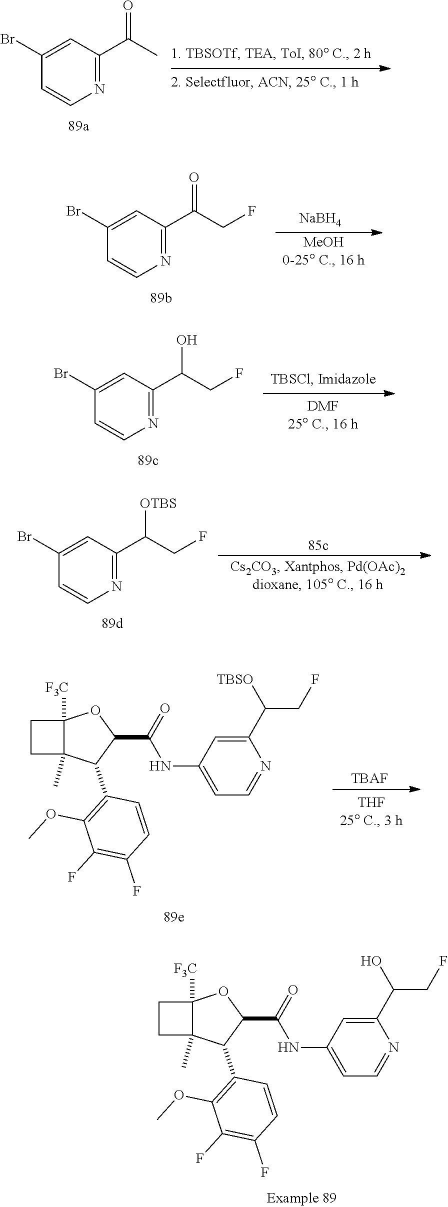

### Step 1: 1-(4-bromopyridin-2-yl)-2-fluoroethan-1-one (89b)

In a reaction flask, the compound 1-(4-bromopyridin-2-yl)ethan-1-one 89a (300 mg, 1.50 mmol, 1.00 eq) was dissolved in toluene (3 mL), and tert-butyldimethylsilyl trifluoromethanesulfonate (595 mg, 2.25 mmol, 1.50 eq) and triethylamine (326 mg, 3.22 mmol, 2.15 eq) were added. The reaction solution was stirred at 80° C. for 2 h. The reaction solution was concentrated, and acetonitrile (3 mL) and Selectfluor (537 mg, 1.51 mmol, 1.01 eq) were added. The reaction solution was stirred at 25° C. for 1 h. After the completion of the reaction of the raw material was monitored by LCMS, the reaction solution was concentrated. The residue was purified by silica gel column chromatography (petroleum ether/ethyl acetate=50/1) to obtain the compound 89b (176 mg, 54% yield), which was a white solid. LCMS: [M+H]⁺=218.0.

### Step 2: 1-(4-bromopyridin-2-yl)-2-fluoroethan-1-ol (89c)

In a reaction flask, the compound 89a (176 mg, 0.81 mmol, 1.00 eq) was dissolved in methanol (5 mL), and sodium borohydride (60 mg, 1.60 mmol, 1.98 eq) was added at 0° C. The reaction solution was stirred at 25° C. for 16 h. After the completion of the reaction of the raw material was monitored by LCMS, the reaction solution was concentrated. The residue was purified by silica gel column chromatography (petroleum ether/ethyl acetate=3/1) to obtain the compound 89c (145 mg, 81% yield), which was a yellow oily substance. LCMS: [M+H]+=220.0.

### Step 3: 4-bromo-2-(1-((tert-butyldimethylsilyl)oxy)-2-fluoroethyl)pyridine (89d)

In a reaction flask, the compound 89c (130 mg, 0.59 mmol, 1.0 eq) was dissolved in N,N-dimethylformamide (5 mL), and imidazole (149 mg, 2.18 mmol, 3.7 eq) and tert-butyldimethylchlorosilane (151 mg, 1.00 mmol, 1.7 eq) were added. The reaction solution was stirred at 25° C. for 16 h. After the completion of the reaction of the raw material was monitored by LCMS, the reaction solution was diluted with water (15 mL), subjected to extraction with ethyl acetate (10 mL×3), washed with saturated saline, dried over anhydrous sodium sulfate, and then concentrated. The residue was purified by silica gel column chromatography (petroleum ether/ethyl acetate=20/1) to obtain the compound 89d (173 mg, 87% yield), which was a colorless oily substance. LCMS: [M+H]⁺=334.0.

### Step 4: rel-(1R,3R,4S,5R)—N-(2-(1-((tert-butyldimethylsilyl)oxy)-2-fluoroethyl)pyridin-4-yl)-4-(3,4-difluoro-2-methoxyphenyl)-5-methyl-1-(trifluoromethyl)-2-oxabicyclo[3.2.0]heptane-3-carboxamide (89e)

In a reaction flask, the compound 89d (151 mg, 0.45 mmol, 1.5 eq) was dissolved in anhydrous 1,4-dioxane (5 mL), and cesium carbonate (196 mg, 0.60 mmol, 2.0 eq), 85c (110 mg, 0.30 mmol, 1.0 eq), 4,5-bis(diphenylphosphino)-9,9-dimethylxanthene (35 mg, 0.06 mmol, 0.2 eq), and palladium acetate (5 mg, 0.03 mmol, 0.1 eq) were added. The reaction solution reacted at 105° C. in a nitrogen atmosphere for 16 h. After the completion of the reaction of the raw material was monitored by LCMS, the reaction solution was diluted with water (15 mL), subjected to extraction with ethyl acetate (10 mL×3), washed with saturated saline, dried over anhydrous sodium sulfate, and then concentrated. The residue was purified by silica gel column chromatography (petroleum ether/ethyl acetate=2/1) to obtain the compound 89e (193 mg, 98% yield), which was a colorless oily substance. LCMS: [M+H]⁺=619.3

### Step 5: rel-(1R,3R,4S,5R)-4-(3,4-difluoro-2-methoxyphenyl)-N-(2-(2-fluoro-1-hydroxyethyl)pyridin-4-yl)-5-methyl-1-(trifluoromethyl)-2-oxabicyclo[3.2.0]heptane-3-carboxamide (Example 89)

The compound 89e (193 mg, 0.31 mmol, 1.0 eq) was dissolved in tetrahydrofuran (3 mL), and a tetrabutylammonium fluoride in tetrahydrofuran solution (0.4 mL) was added. The reaction solution reacted at 25° C. for 3 h. After the completion of the reaction of the raw material was monitored by LCMS, the reaction solution was diluted with water (10 mL), subjected to extraction with ethyl acetate (10 mL×3), washed with saturated saline, dried over anhydrous sodium sulfate, and then concentrated. The residue was purified by silica gel column chromatography (petroleum ether/ethyl acetate=1/1) and preparative separation (0.1% FA) to obtain the compound Example 89 (68.5 mg, 41.3% yield), which was a white solid compound. LCMS: [M+H]⁺=505.1. ¹H NMR (400 MHz, DMSO-d₆) δ 10.64 (d, J=3.6 Hz, 1H), 8.39 (d, J=5.6 Hz, 1H), 7.85 (t, J=2.4 Hz, 1H), 7.61 (d, J=5.6 Hz, 1H), 7.19 (dd, J=16.8, 8.8 Hz, 1H), 7.12-7.06 (m, 1H), 5.95 (s, 1H), 5.11 (d, J=8.4 Hz, 1H), 4.80 (d, J=19.6 Hz, 1H), 4.66 (ddd, J=47.6, 9.6, 2.8 Hz, 1H), 4.50 (ddd, J=47.6, 9.2, 6.0 Hz, 1H), 4.23 (d, J=8.4 Hz, 1H), 3.93 (d, J=2.0 Hz, 3H), 2.46-2.35 (m, 2H), 2.21-2.13 (m, 1H), 1.87-1.78 (m, 1H), 0.81 (s, 3H).

### Example 90: Synthesis of Compound 90

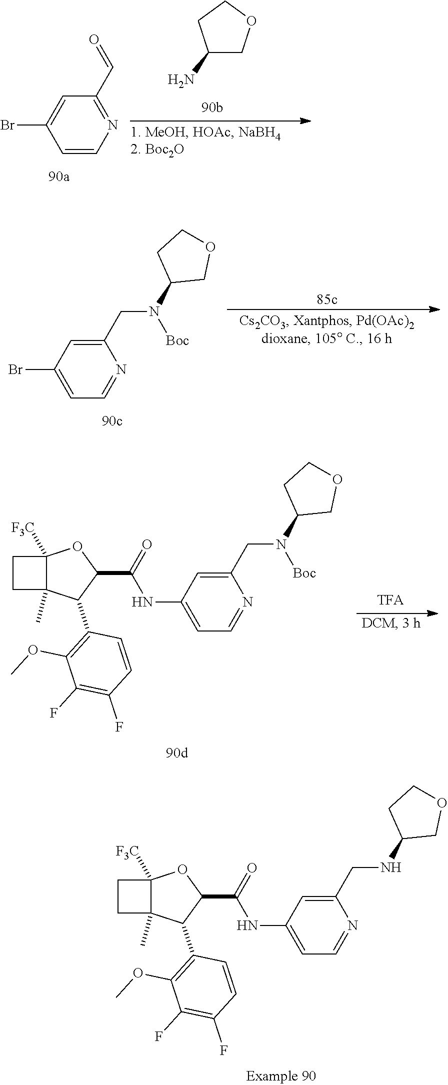

### Step 1: (S)-(4-bromopyridin-2-yl)methyl(tetrahydrofuran-3-yl)tert-butyl carbamate (90c)

In a single-necked flask, the compound 4-bromopyridine carbaldehyde 90a (100 mg, 0.54 mmol, 1.0 eq) was dissolved in 1,2-dichloroethane (5 mL), and (S)-tetrahydrofuran-3-amine 90b (61 mg, 0.70 mmol, 1.3 eq), acetic acid (32.38 mg, 0.54 mmol, 1.0 eq), and sodium triacetoxyborohydride (171 mg, 0.81 mmol, 1.5 eq) were added. The reaction solution was stirred at 25° C. for 3 h. Then, di-tert-butyl dicarbonate (176 mg, 0.81 mmol, 1.5 eq) was added to the reaction solution. The reaction solution was stirred at 25° C. for 0.5 h. After the completion of the reaction of the raw material was monitored by LCMS, the reaction solution was concentrated. The residue was purified by silica gel column chromatography (petroleum ether/ethyl acetate=4/1) to obtain the compound 90c (170 mg, 88% yield), which was a yellow oily substance.

### Step 2: rel-tert-butyl-(4-(1R,3R,4S,5R)-4-(3,4-difluoro-2-methoxyphenyl)-5-methyl-1-trifluoromethyl-2-oxabicyclo[3.2.0]heptane-3-carboxamido)pyridin-2-yl)methyl((S)-tetrahydrofuran-3-yl)carbamate (90d)

In a single-necked flask, the compound 90c (65 mg, 0.18 mmol, 1.0 eq) was dissolved in anhydrous 1,4-dioxane (5 mL), and cesium carbonate (107 mg, 0.33 mmol, 2.0 eq), the compound 85c (60 mg, 0.16 mmol, 1.0 eq), 4,5-bis(diphenylphosphino)-9,9-dimethylxanthene (19 mg, 0.03 mmol, 0.2 eq), and palladium acetate (2.7 mg, 0.02 mmol, 0.1 eq) were added. The reaction solution reacted at 105° C. in a nitrogen atmosphere for 16 h. After the completion of the reaction of the raw material was monitored by LCMS, the reaction solution was diluted with water (15 mL), subjected to extraction with ethyl acetate (10 mL×3), washed with saturated saline, dried over anhydrous sodium sulfate, and then concentrated. The residue was purified by silica gel column chromatography (petroleum ether/ethyl acetate=20/1) to obtain the compound 90d (30 mg, 29% yield), which was a yellow oily substance.

### Step 3: rel-(1R,3R,4S,5R)-4-(3,4-difluoro-2-methoxyphenyl)-5-methyl-N-(2-((S)-tetrahydrofuran-3-amino)methyl)pyridin-4-yl)-1-trifluoromethyl-2-oxabicyclo[3.2.0]heptane-3-carboxamide (Example 90)

The compound 90d (30 mg, 0.05 mmol, 1.0 eq) was dissolved in dichloromethane (2 mL), and trifluoroacetic acid (1 mL) was added. The reaction solution reacted at 25° C. for 3 h. After the completion of the reaction of the raw material was monitored by LCMS, the reaction mixture was concentrated. The residue was purified by preparative separation (0.1% FA) to obtain Example 90 (1.7 mg, 6% yield), which was a white solid. LCMS: [M+H]⁺=542.2. ¹H NMR (400 MHz, CDCl₃) δ 8.68 (s, 1H), 8.46 (d, J=5.6 Hz, 1H), 7.59 (s, 1H), 7.52 (d, J=4.8 Hz, 1H), 6.96 (d, J=5.6 Hz, 2H), 6.93-6.87 (m, 1H), 5.02 (d, J=6.4 Hz, 1H), 4.20 (s, 1H), 4.00 (d, J=2.4 Hz, 3H), 3.96 (s, 2H), 3.86-3.72 (m, 4H), 3.55 (s, 1H), 2.59-2.52 (m, 1H), 2.25-2.14 (m, 3H), 1.91-1.90 (m, 1H), 0.90 (s, 3H).

### Example 91: rel-(1R,3R,4S,5R)-4-(3,4-difluoro-2-methoxyphenyl)-N-(2-methoxyethylamino)methylpyridin-4-yl)-5-methyl-1-trifluoromethyl-2-oxabicyclo[3.2.0]heptane-3-carboxamide (91)

Example 91 was prepared by a similar method with 2-methoxyethan-1-amine that replaced 90b in Example 90. LCMS: [M+H]⁺=530.2. ¹H NMR (400 MHz, CDCl₃) δ 8.64 (s, 1H), 8.47 (d, J=5.2 Hz, 1H), 7.57-7.53 (m, 2H), 6.97-6.86 (m, 2H), 5.01 (d, J=6.8 Hz, 1H), 4.20 (s, 1H), 4.00 (s, 3H), 3.58 (t, J=4.8 Hz, 2H), 3.38 (s, 3H), 2.95 (t, J=4.8 Hz, 2H), 2.57-2.50 (m, 1H), 2.19-2.10 (m, 3H), 1.94-1.89 (m, 2H), 0.90 (s, 3H).

### Example 92

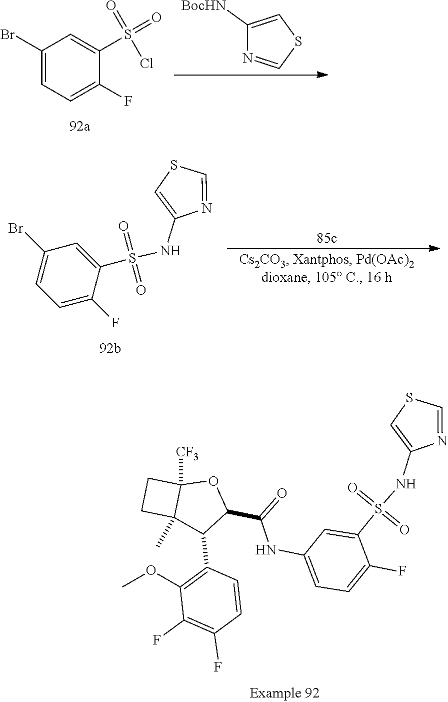

### Step 1: 5-bromo-2-fluoro-N-(thiazol-4-yl)benzenesulfonamide (92b)

4-bromo-2-fluorobenzenesulfonyl chloride 92a (200 mg, 1.0 eq) was dissolved in THF (5 mL) and cooled down to 0° C., and NaHMDS (0.9 mL, 1.0 M, 1.2 eq) was added dropwise under nitrogen protection. After 1 h, a thiazole-4-tert-butyl carbamate (161 mg, 1.1 eq) in THF (1 mL) solution was added. After the reaction system was warmed slowly to room temperature, a saturated ammonium chloride solution was added. Extraction was carried out with ethyl acetate (20 mL×3). The organic phases were combined, washed with saline, dried over sodium sulfate, filtered, spin-dried, and purified by silica gel column chromatography (petroleum ether/ethyl acetate=3/1), to obtain 5-bromo-2-fluoro-N-(thiazol-4-yl)benzenesulfonamide 92b (150 mg, 60% yield), which was a white solid. LCMS: [M+H]⁺=336.8, 338.8.

### Step 2: rel-(1R,3R,4S,5R)-4-(3,4-difluoro-2-methoxyphenyl)-N-(4-fluoro-3-(N-(thiazol-4-yl)aminosulfonyl)phenyl)-5-methyl-1-trifluoromethyl-2-oxabicyclo[3.2.0]heptane-3-carboxamide (Example 92)

85c (30 mg, 0.08 mmol, 1.0 eq) was dissolved in 1,4-dioxane (5 mL), and 92b (42 mg, 0.12 mmol, 1.5 eq), 4,5-bis(diphenylphosphino)-9,9-dimethylxanthene (9.5 mg, 0.016 mmol, 0.2 eq), cesium carbonate (80 mg, 0.25 mmol, 3 eq), and palladium acetate (1.8 mg, 0.008 mmol, 0.1 eq) were added. The mixture was stirred at 105° C. for 16 h. The filtrate was concentrated under reduced pressure, and purified by preparative separation (0.1% FA), to obtain the compound Example 92 (2.4 mg, 4% yield), which was a white solid. LCMS: [M+H]⁺=622.1. ¹H NMR (400 MHz, MeOD) δ 8.53 (s, 1H), 8.01-8.00 (m, 1H), 7.74-7.72 (m, 1H), 7.10-7.06 (m, 1H), 6.99-6.96 (m, 1H), 6.92-6.86 (m, 1H), 6.67 (s, 1H), 4.95-4.93 (m, 1H), 4.21-4.19 (m, 1H), 3.87 (s, 3H), 2.41-2.22 (m, 2H), 2.14-2.07 (m, 1H), 1.81-1.74 (m, 1H), 0.80 (s, 3H).

### Example 93: Synthesis of Compound 93

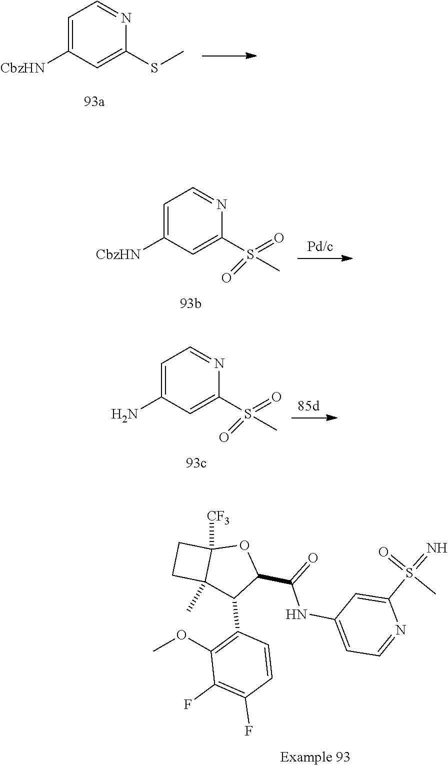

### Step 1: (2-(S-methylsulfoximine)pyridin-4-yl)benzyl carbamate (93b)

93a (2.2 g, 8.0 mmol, 1.0 eq) was weighed and added in a reaction flask, and (diacetoxyiodo)benzene (8.3 g, 25.7 mmol, 3.2 eq), aminocarboxamide (4.3 g, 56.2 mmol, 7.0 eq), dichloromethane (150 mL), and methanol (150 mL) were added. The reaction system reacted at room temperature for 16 h, until the completion of the reaction was monitored by LCMS. The reaction solution was concentrated and then purified by column chromatography (petroleum ether:ethyl acetate=0-100%) to obtain 93b, which was a yellow solid (2.3 g, 94% yield). LCMS: [M+H]⁺=306.4.

### Step 2: (4-aminopyridin-2-yl)(imino)(methyl)-1-6-sulfonic acid ketone (93c)

The compound 93b (300 mg, 0.98 mmol, 1.0 eq) was dissolved in methanol (10 mL), and palladium on carbon (314 mg, 2.95 mmol, 3.0 eq) was added. The reaction solution reacted at 25° C. in hydrogen for 16 h, until the completion of the reaction was monitored by LCMS. The reaction solution was filtered. The filtrate was collected and concentrated to obtain a residue. The residue was purified by silica gel column chromatography (dichloromethane/methanol=10/1) to obtain the compound 93c (100 mg, 59% yield), which was a white solid. LCMS: [M+H]⁺=172.1.

### Step 3: rel-(1R,3R,4S,5R)-4-(3,4-difluoro-2-methoxyphenyl)-5-methyl-N-(2-(S-methylsulfoximine)pyridin-4-yl)-1-(trifluoromethyl)-2-oxabicyclo[3.2.0]heptane-3-carboxamide (Example 93)

The compound 85d (50 mg, 0.14 mmol, 1.0 eq) was dissolved in dichloromethane (10 mL), and the compound 93c (28 mg, 0.16 mmol, 1.2 eq), benzotriazole-N,N,N′,N′-tetramethyluronium hexafluorophosphate (62 mg, 0.16 mmol, 1.2 eq), and N,N-diisopropylethylamine (35 mg, 0.27 mmol, 2.0 eq) were added. The reaction solution was stirred at 25° C. for 2 h, until the completion of the reaction was monitored by LCMS. The reaction solution was concentrated to obtain a residue. The residue was purified by preparative separation (0.01% FA) to obtain Example 93 (8.2 mg, 11.6% yield), which was a white solid. LCMS: [M+H]⁺=520.1. ¹H NMR (400 MHz, CD₃OD) δ 8.06 (d, J=5.6 Hz, 1H), 7.33 (s, 1H), 7.01-6.96 (m, 2H), 6.71-6.68 (m, 1H), 4.92 (d, J=5.6 Hz, 1H), 4.88-4.84 (m, 3H), 4.19 (s, 1H), 3.95-3.93 (m, 3H), 2.45-2.18 (m, 2H), 2.18-1.97 (m, 1H), 1.79-1.73 (m, 1H), 0.85 (d, J=5.2 Hz, 3H).

### Example 94: Synthesis of Compound 94

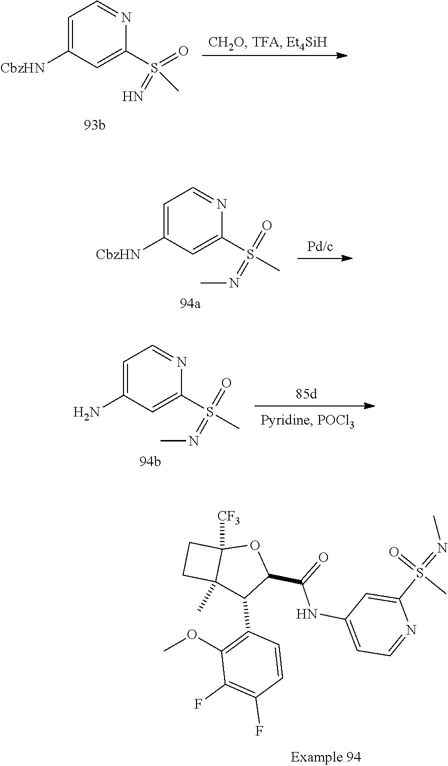

### Step 1: (2-(N,S-dimethylsulfoximine)pyridin-4-yl)benzyl carbamate (94a)

The compound 93b (300 mg, 0.98 mmol, 1.0 eq) was dissolved in acetonitrile (20 mL), and tetraethylsilane (283 mg, 1.96 mmol, 2.0 eq), formaldehyde (294 mg, 4.91 mmol, 5.0 eq), and trifluoroacetic acid (233 mg, 2.94 mmol, 3.0 eq) were added. The reaction solution reacted at 25° C. for 16 h. until the completion of the reaction was monitored by LCMS. The reaction solution was concentrated to obtain a residue. The residue was purified by silica gel column chromatography (dichloromethane/methanol=30/1) to obtain the compound 94a (300 mg, 95% yield), which was a colorless oily substance. LCMS: [M+H]⁺=320.1.

### Step 2: (4-aminopyridin-2-yl)(methyl)(methylimino)-1-6-sulfonic acid ketone (94b)

The compound 94a (300 mg, 0.94 mmol, 1.0 eq) was dissolved in methanol (10 mL), and palladium on carbon (300 mg, 2.82 mmol, 3.0 eq) was added. The reaction solution reacted at 25° C. in hydrogen for 16 h, until the completion of the reaction was monitored by LCMS. The reaction solution was filtered. The filtrate was collected and concentrated to obtain a residue. The residue was purified by silica gel column chromatography (dichloromethane/methanol=10/1) to obtain the compound 94b (150 mg, 86% yield), which was a white solid. LCMS: [M+H]⁺=186.1.

### Step 3: rel-(1R,3R,4S,5R)-4-(3,4-difluoro-2-methoxyphenyl)-N-(2-(N,S-dimethylsulfonyl)pyridin-4-yl)-5-methyl-1-(trifluoromethyl)-2-oxabicyclo[3.2.0]heptane-3-carboxamide (Example 94)

The compound 85d (50 mg, 0.14 mmol, 1.0 eq) was dissolved in anhydrous pyridine (3 mL), the compound 94b (30 mg, 0.16 mmol, 1.2 eq) was added and mixed uniformly, and then phosphorus oxychloride (84 mg, 0.55 mmol, 4.0 eq) was added at 0° C. The reaction solution was stirred at 0° C. for 15 min, until the completion of the reaction was monitored by LCMS. The reaction solution was poured into water and subjected to extraction with ethyl acetate. The organic phase was collected. The organic phase was dried over anhydrous sodium sulfate and concentrated to obtain a residue. The residue was purified by preparative separation (0.01% FA) to obtain the compound Example 94 (14.2 mg, 16% yield), which was a white solid. LCMS: [M+H]⁺=534.0. ¹H NMR (400 MHz, CD₃OD) δ 8.62 (d, J=5.2 Hz, 1H), 8.48 (t, J=1.6 Hz, 1H), 7.95 (dd, J=2.0, 2.0 Hz, 1H), 7.11-7.04 (m, 1H), 7.02-6.95 (m, 1H), 5.11 (d, J=8.0 Hz, 1H), 4.34 (d, J=7.6 Hz, 1H), 3.98 (t, J=2.4 Hz, 3H), 3.22 (s, 3H), 2.57 (s, 3H), 2.52-2.45 (m, 1H), 2.39-2.31 (m, 1H), 2.22-2.17 (m, 1H), 1.93-1.84 (m, 1H), 0.90 (d, J=1.2 H, 3H).

### Example 95: Synthesis of Compound 95

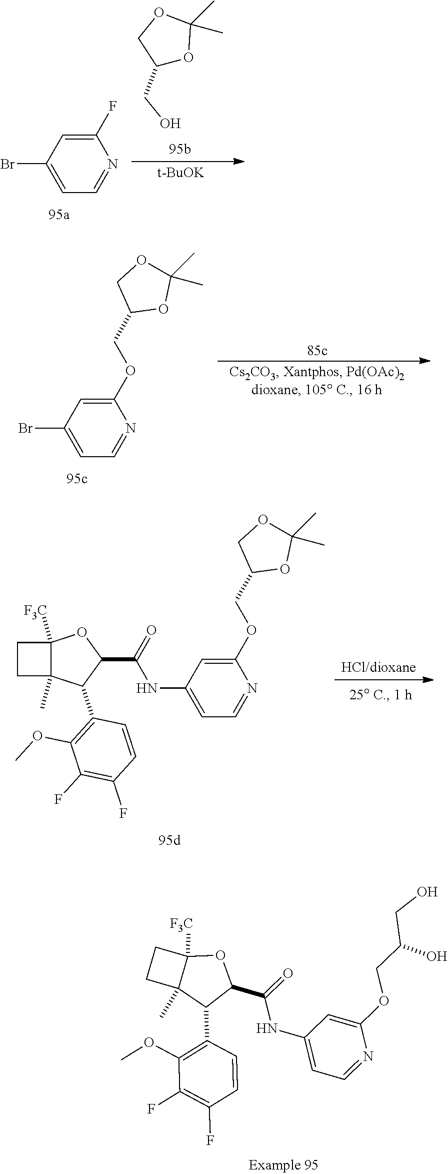

### Step 1: R-4-bromo-2-(2,2-dimethyl-1,3-dioxolan-4-yl)methoxy)pyridine (95c)

4-bromo-2-fluoropyridine 95a (1.0 g, 5.7 mmol, 1.0 eq) and R-2,2-dimethyl-1,3-dioxolan-4-methanol 95b (0.9 g, 6.8 mmol, 1.2 eq) were dissolved in hydrochloric acid/1,4-dioxane (10 mL), and potassium tert-butoxide (1.9 g, 17.1 mmol, 0.1 eq), 1,1′-binaphthyl-2,2′-bisdiphenylphosphine (0.4 g, 0.5 mmol, 0.1 eq), and palladium acetate (0.13 g, 0.5 mmol, 0.1 eq) were added. The reaction solution was stirred at 105° C. for 16 h. After the completion of the reaction of the raw material was monitored by LCMS, the reaction solution was quenched with 1 M dilute hydrochloric acid (4 mL), subjected to extraction with dichloromethane (10 mL×3), washed with saturated saline, dried over anhydrous sodium sulfate, and then concentrated, to obtain the compound 95c (0.5 g, 30% yield), which was a white solid. LCMS: [M+H]⁺=288.0

### Step 2: rel-(1R,3R,4S,5R)-4-(3,4-difluoro-2-methoxyphenyl)-N-(2,2-dimethyl-1,3-dioxolan-4-yl)methoxy)pyridin-4-yl)-5-methyl-1-trifluoromethyl-2-oxabicyclo[3.2.0]heptane-3-carboxamide (95d)

85c (500 mg, 1.37 mmol, 1.0 eq) was dissolved in a 1,4-dioxane solution, and (R)-4-bromo-2-((2,2-dimethyl-1,3-dioxolan-4-yl)methoxy)pyridine 96b (592 mg, 2.0 mmol, 1.5 eq), 4,5-bis(diphenylphosphino)-9,9-dimethylxanthene (158 mg, 0.27 mmol, 0.2 eq), cesium carbonate (1.3 g, 4.1 mmol, 3.0 eq), and palladium acetate (31 mg, 0.14 mmol, 0.1 eq) were added. The reaction solution was stirred in nitrogen at 105° C. for 16 h. After the reaction, the mixture was filtered. The filtrate was concentrated under reduced pressure to obtain a residue. The residue was purified by column chromatography (ethyl acetate:petroleum ether=1:3) to obtain the compound 95d (100 mg, 30% yield), which was a yellow solid. LCMS: [M+H]⁺=573.2.

### Step 3: rel-(1R,3R,4S,5R)-4-(3,4-difluoro-2-methoxyphenyl)-N-(2-(S)-2,3-dihydroxypropoxypyridin-4-yl)-5-methyl-1-trifluoromethyl-2-oxabicyclo[3.2.0]heptane-3-carboxamide (Example 95)

A mixture of 95d (100 mg, 0.17 mmol, 1.0 eq) was stirred at 25° C. for 1 h, until the completion of the reaction was monitored by LCMS. The mixture was concentrated under reduced pressure. The compound was purified by preparative separation (0.1% FA) to obtain Example 95 (23.4 mg, 25% yield), which was a white solid. LCMS: [M+H]⁺=533.2. ¹H NMR (400 MHz, DMSO-d₆) δ 10.52 (s, 1H), 8.02 (d, J=8.0 Hz, 1H), 7.22-7.17 (m, 3H), 7.10-7.06 (m, 1H), 5.10 (d, J=8.0 Hz, 1H), 4.88 (d, J=8.0 Hz, 1H), 4.61 (t, J=8.0 Hz, 1H), 4.25-4.21 (m, 2H), 4.12-4.08 (m, 1H), 3.93 (d, J=4.0 Hz, 3H), 3.77-3.73 (m, 1H), 3.41 (t, J=4.0 Hz, 2H), 2.45-2.36 (m, 1H), 2.32-2.29 (m, 1H), 2.19-2.12 (m, 1H), 1.86-1.79 (m, 1H), 0.81 (s, 3H).

### Example 96: rel-(1R,3R,4S,5R)-4-(3,4-difluoro-2-methoxyphenyl)-N-(2-(R)-2,3-dihydroxypropoxypyridin-4-yl)-5-methyl-1-trifluoromethyl-2-oxabicyclo[3.2.0]heptane-3-carboxamide (96)

Example 96 was prepared by a similar method with S-2,2-dimethyl-1,3-dioxolan-4-methanol that replaced 95b in Example 95. LCMS: [M+H]⁺=533.1. ¹H NMR (400 MHz, DMSO-d₆) δ 10.55 (s, 1H), 8.02 (d, J=5.7 Hz, 1H), 7.24-7.15 (m, 3H), 7.12-7.06 (m, 1H), 5.11 (d, J=8.0 Hz, 1H), 4.23 (dd, J=9.7, 4.5 Hz, 2H), 4.10 (dd, J=10.9, 6.3 Hz, 1H), 3.93 (d, J=2.2 Hz, 3H), 3.77-3.59 (m, 5H), 2.43-2.29 (m, 2H), 2.20-2.11 (m, 1H), 1.83 (td, J=10.7, 7.0 Hz, 1H), 0.81 (s, 3H).

### Example 97: 5-((1S,3R,4S,5S)-4-(3,4-difluoro-2-methoxyphenyl)-5-methyl-1-(trifluoromethyl)-2-oxabicyclo[3.2.0]heptane-3-formylamino)pyridinecarboxamide (97)

Example 97 was prepared by a similar method with 5-aminopicolinamide that replaced 5-amino-2-fluorobenzamide in Example 85. LCMS: [M+H]⁺=486.1. ¹H NMR (400 MHz, DMSO-d₆) δ 10.68 (s, 1H), 8.87 (d, J=2.0 Hz, 1H), 8.25 (dd, J=8.6, 2.5 Hz, 1H), 8.01 (d, J=8.6 Hz, 2H), 7.55 (s, 1H), 7.22-7.07 (m, 2H), 5.16 (d, J=8.1 Hz, 1H), 4.25 (d, J=8.1 Hz, 1H), 3.92 (dd, J=8.0, 2.1 Hz, 4H), 2.20-1.81 (m, 2H), 0.82 (s, 4H).

### Example 98: Synthesis of Compound 98

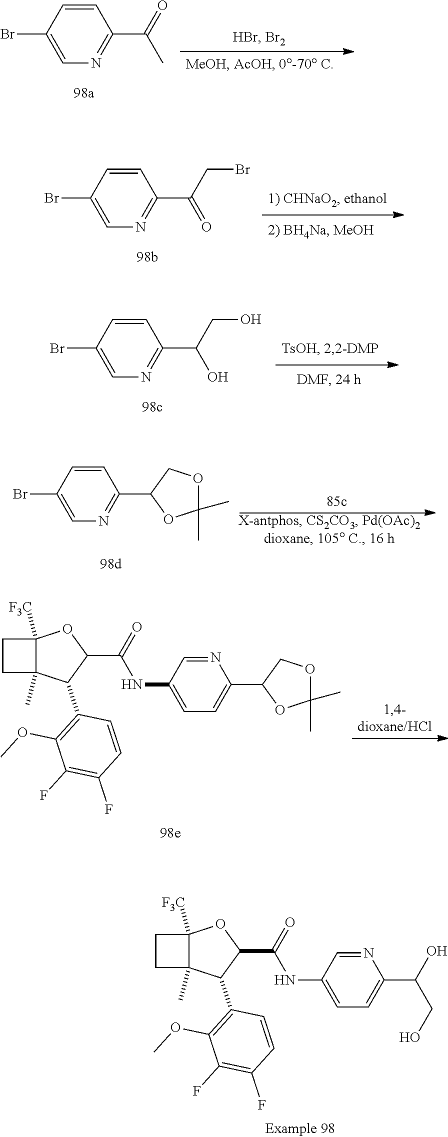

### Step 1: 2-bromo-1-(5-bromopyridin-2-yl)ethan-1-one (98b)

1-(5-bromopyridin-2-yl)ethanone 98a (2.0 g, 0.01 mmol, 1.0 eq) was dissolved in methanol (10 mL) and acetic acid (15 mL) and cooled down to 0° C. A 30% hydrogen bromide in acetic acid solution (2.8 mL) was added. Bromine (1.6 g, 0.01 mmol, 1.0 eq) in acetic acid (5 mL) was added dropwise to warm the reaction mixture to room temperature, and then heated to 70° C. and stirred for 1 h, until the completion of the reaction was monitored by LCMS. The mixture was concentrated under reduced pressure. The residue was crystallized with isopropanol to obtain 98b (1.0 g, 32% yield), which was a yellow solid. LCMS: [M+H]⁺=278.9.

### Step 2: 1-(5-bromopyridin-2-yl)ethan-1,2-diol (98c)

An ethanol solution (10 mL) of 2-bromo-1-(5-bromopyridin-2-yl)ethanone 98b (1.0 g, 3.6 mmol, 1.0 eq) and sodium formate (1.0 g, 14.4 mmol, 4.0 eq) was heated to 50° C. to react for 3 h. After the reaction, the reaction system was cooled down to room temperature, and the solvent was evaporated under reduced pressure. The mixture was quenched with water (10 mL), and subjected to extraction with dichloromethane (50 mL×3). The organic phases were combined, dried over sodium sulfate, and filtered. The filtrate was concentrated under reduced pressure. The residue was absorbed in methanol (10 mL) and cooled down to 0° C. Sodium borohydride (0.4 g, 10.8 mmol, 3.0 eq) was added in batches, and the mixture was stirred at 0° C. for 1 h. Concentrated hydrochloric acid was added to adjust pH to 2, and the solvent was removed under reduced pressure. Extraction with dichloromethane (50 mL) and saturated aqueous sodium bicarbonate (10 mL) was carried out for three times. The organic phases were combined, and dried over sodium sulfate. The residue was purified by silica gel column chromatography (petroleum ether/ethyl acetate=4/1) to obtain the compound 98c (350 mg, 42% yield), which was a yellow solid. LCMS: [M+H]⁺=218.0.

### Step 3: 5-bromo-2-(2,2-dimethyl-1,3-dioxolan-4-yl)pyridine (98d)

1-(5-bromopyridin-2-yl)ethan-1,2-diol 98c (100 mg, 0.46 mmol, 1.0 eq) was dissolved in N,N-dimethylformamide (5 mL), and 2,2-dimethoxypropane (48 mg, 0.46 mmol, 1.0 eq) and p-toluenesulfonic acid (7.9 mg, 0.05 mmol, 0.1 eq) were added. The mixture was stirred at 25° C. for 24 h, until the completion of the reaction was monitored by LCMS. The mixture was quenched with water (50 mL), and then subjected to extraction with dichloromethane (50 mL×3). The organic phases were combined, dried over sodium sulfate, and filtered. The filtrate was concentrated under reduced pressure. The residue was purified by elution with ethyl acetate in petroleum ether in 0-50% to obtain 5-bromo-2-(2,2-dimethyl-1,3-dioxolan-4-yl)pyridine 98d (60 mg, 48% yield), which was a yellow oily substance. LCMS: [M+H]⁺=258.0.

### Step 4: rel-(1R,3R,4S,5R)-4-(3,4-difluoro-2-methoxyphenyl)-N-(6-(2,2-dimethyl-1,3-dioxolan-4-yl)pyridin-3-yl)-5-methyl-1-trifluoromethyl-2-oxabicyclo[3.2.0]heptane-3-carboxamide (98e)

85c (50 mg, 0.14 mmol, 1.0 eq) was dissolved in 1,4-dioxane (5 mL), and 5-bromo-2-(2,2-dimethyl-1,3-dioxolan-4-yl)pyridine (53 mg, 0.21 mmol, 1.5 eq), 4,5-bis(diphenylphosphino)-9,9-dimethylxanthene (16 mg, 0.027 mmol, 0.2 eq), cesium carbonate (134 mg, 0.41 mmol, 3.0 eq), and palladium acetate (3.1 mg, 0.014 mmol, 0.1 eq) were added. The mixture was stirred at 105° C. for 16 h. After the reaction, the mixture was quenched with water (50 mL), and then subjected to extraction with ethyl acetate (50 mL×3). The organic phases were combined, dried over sodium sulfate, and filtered. The filtrate was concentrated under reduced pressure, and purified by silica gel column chromatography (petroleum ether/ethyl acetate=3/1) to obtain 98e (50 mg, 67% yield), which was a yellow oily substance. LCMS: [M+H]⁺=543.2.

### Step 5: rel-(1R,3R,4S,5R)-4-(3,4-difluoro-2-methoxyphenyl)-N-(6-(1,2-dihydroxyethyl)pyridin-3-yl)-5-methyl-1-trifluoromethyl-2-oxabicyclo[3.2.0]heptane-3-carboxamide (Example 98)

98e (50 mg, 0.0184 mmol, 1.0 eq) was dissolved in hydrochloric acid/1,4-dioxane (5 mL), and stirred at 25° C. for 1 h. The mixture was concentrated under reduced pressure, and purified by preparative separation (0.1% NH₃), to obtain the compound Example 98 (4.2 mg, 9% yield), which was a white solid. LCMS: [M+H]⁺=503.2. ¹H NMR (400 MHz, CD₃OD) δ 9.15 (s, 1H), 8.49-8.47 (m, 1H), 7.89-7.87 (m, 1H), 7.12-7.07 (m, 1H), 7.03-6.96 (m, 1H), 5.15-5.14 (m, 1H), 4.95-4.93 (m, 1H), 4.34-4.31 (m, 1H), 3.98-3.97 (m, 3H), 3.84-3.76 (m, 2H), 2.54-2.46 (m, 1H), 2.40-2.33 (m, 1H), 2.25-2.17 (m, 1H), 1.93-1.86 (m, 1H), 0.91 (s, 3H).

### Example 99: Synthesis of Compound 99

Example 99 was prepared by a similar method with 5-amino-N,N-bis(3,4-dimethoxybenzyl)pyridin-2-sulfonamide that replaced 34e in Example 87. LCMS: [M+H]⁺=522.1. ¹H NMR (400 MHz, DMSO-d₆) δ 10.73 (s, 1H), 8.93 (d, J=2.4 Hz, 1H), 8.31 (dd, J=8.4, 2.4 Hz, 1H), 7.90 (d, J=8.8 Hz, 1H), 7.40 (s, 2H), 7.23-7.16 (m, 1H), 7.13-7.09 (m, 1H), 5.17 (d, J=8.0 Hz, 1H), 4.25 (d, J=8.0 Hz, 1H), 3.93 (d, J=2.4 Hz, 3H), 2.43-2.36 (m, 2H), 2.19-2.13 (m, 1H), 1.87-1.80 (m, 1H), 0.83 (s, 3H).

### Example 100: Synthesis of Compound 100

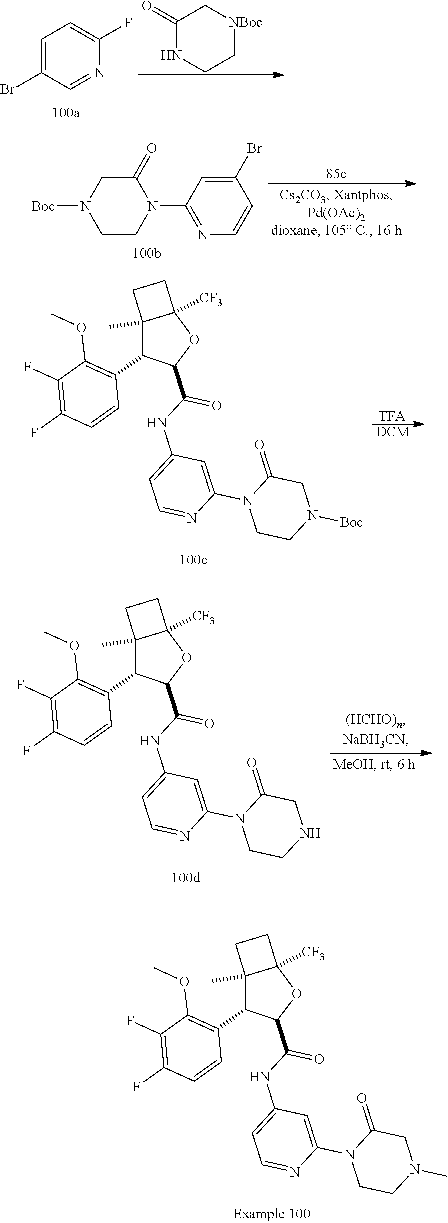

### Step 1: tert-butyl 4-(5-bromopyridin-2-yl)-3-oxopiperidin-1-carboxylate (100b)

1-Boc-3-oxopiperazine (1.37 g, 6.82 mmol, 1.2 eq) was dissolved in DMF (15 mL) and cooled down to 0° C., and sodium hydride (341 mg, 8.52 mmol, 1.5 eq) was added and stirred for 30 min. 2-fluoro-5-bromopyridine 100a (1.0 g, 5.68 mmol, 1.0 eq) was added, heated to 60° C., and stirred for 16 h. The reaction system with water added was subjected to extraction with ethyl acetate (50 mL×3). The organic phases were combined, washed with saline, dried over sodium sulfate, filtered, spin-dried, and purified by silica gel column chromatography (petroleum ether/ethyl acetate=3/1), to obtain tert-butyl 4-(5-bromopyridin-2-yl)-3-oxopiperidin-1-carboxylate 100b (560 mg, 27.7% yield), which was a white solid. LCMS: [M+H]⁺=355.9, 357.9.

### Step 2: rel-tert-butyl 4-(4-((1R,3R,4S,5R)-4-(3,4-difluoro-2-methoxyphenyl)-5-methyl-1-(trifluoromethyl)-2-oxabicyclo[3.2.0]heptane-3-carboxamido)pyridin-2-yl)-3-oxopiperazin-1-carboxylate (100c)

The compound 85c (50 mg, 0.14 mmol, 1.0 eq) was dissolved in dioxane (5 mL), and 100b (65 mg, 0.18 mmol, 1.3 eq), cesium carbonate (133 mg, 0.41 mmol, 3.0 eq), 4,5-bis(diphenylphosphino)-9,9-dimethylxanthene (11 mg, 0.03 mmol, 0.2 eq), and palladium acetate (3 mg, 0.01 mmol, 0.1 eq) were added. The reaction solution reacted at 105° C. in a nitrogen atmosphere for 16 h. After the completion of the reaction of the raw material was monitored by LCMS, the reaction mixture was concentrated. The residue was purified by silica gel column chromatography (petroleum ether/ethyl acetate=10/1) to obtain the compound 100c (50 mg, 57% yield), which was a colorless oily substance.

### Step 3: rel-(1R,3R,4S,5R)-4-(3,4-difluoro-2-methoxyphenyl)-5-methyl-N-(2-(2-oxopiperazin-1-yl)pyridin-4-yl)-1-(trifluoromethyl)-2-oxabicyclo[3.2.0]heptane-3-carboxamide (100d)

The compound 100c (50 mg, 0.078 mmol, 1.0 eq) was dissolved in trifluoroacetic acid (5 mL). The reaction solution reacted at 25° C. for 2 h. After the completion of the reaction of the raw material was monitored by LCMS, the reaction mixture was concentrated to obtain the compound 100d (40 mg, 95% yield), which was a white solid.

### Step 4: rel-(1R,3R,4S,5R)-4-(3,4-difluoro-2-methoxyphenyl)-5-methyl-N-(2-(4-methyl-2-oxopiperazin-1-yl)pyridin-4-yl)-1-(trifluoromethyl)-2-oxabicyclo[3.2.0]heptane-3-carboxamide (Example 100)

The compound 100d (40 mg, 0.07 mmol, 1.0 eq) and paraformaldehyde (4 mg, 0.14 mmol, 2.0 eq) were dissolved in methanol (5 mL). The reaction solution reacted at 25° C. for 30 min. Sodium cyanoborohydride (8 mg, 0.14 mmol, 2.0 eq) was added. The reaction solution reacted at 40° C. for 1 h. After the completion of the reaction of the raw material was monitored by LCMS, the reaction mixture was concentrated. The residue was purified by preparative separation (0.1% FA) to obtain Example 100 (6.7 mg, 13% yield), which was a white solid. LCMS: [M+H]⁺=555.2. ¹H NMR (400 MHz, MeOD) δ 8.33 (d, J=5.6 Hz, 1H), 8.01 (d, J=1.6 Hz, 1H), 7.64 (dd, J=5.6, 2.0 Hz, 1H), 7.16-6.90 (m, 2H), 5.07 (d, J=8.0 Hz, 1H), 4.33 (d, J=8.0 Hz, 1H), 4.00-3.86 (m, 5H), 3.28 (s, 2H), 2.92-2.80 (m, 2H), 2.50-2.43 (m, 1H), 2.40 (s, 3H), 2.35-2.17 (m, 2H), 1.87 (td, J=10.8, 6.4 Hz, 1H), 0.90 (s, 3H).

### Example 101: Synthesis of Compound 101

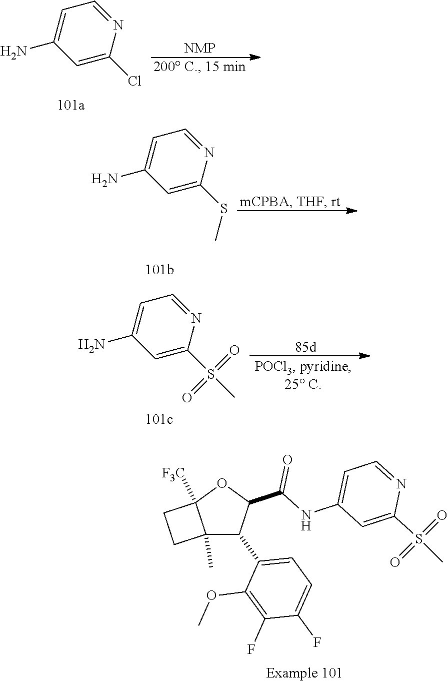

### Step 1: 2-(methylthio)pyridin-4-amine (101b)

Sodium thiomethoxide (1.9 g, 27.0 mmol, 1.7 eq) was dissolved in 1-methylpyrrolidone (10 mL), and 2-chloropyridin-4-amine 101a (2.0 g, 16.0 mmol, 1.0 eq) was added. The reaction system was sealed and heated in a microwave oven to 200° C. to react for 15 min, and then purified by rapid chromatography on silica gel through elution with 8% methanol/dichloromethane to obtain 2-(methylthio)pyridin-4-amine 101b (1.5 g, 55% yield), which was a yellow solid. LCMS: [M+H]⁺=141.0.

### Step 2: 2-(methylsulfonyl)pyridin-4-amine (101c)

2-(methylthio)pyridin-4-amine (100 mg, 0.7 mmol, 1.0 eq) was dissolved in tetrahydrofuran (5 mL), and meta-chloroperbenzoic acid (369 mg, 2.1 mmol, 3.0 eq) was added. The mixture was stirred at 25° C. for 16 h. After the reaction, the mixture was concentrated under reduced pressure to obtain a mixture. The mixture was purified by column chromatography (dichloromethane/methanol=10/1) to obtain 2-(methylsulfonyl)pyridin-4-amine 101c (30 mg, 22% yield), which was a yellow solid. LCMS: [M+H]⁺=173.0.

### Step 3: rel-(1R,3R,4S,5R)-4-(3,4-difluoro-2-methoxyphenyl)-5-methyl-N-(2-methylsulfonyl)pyridin-4-yl)-1-trifluoromethyl-2-oxabicyclo[3.2.0]heptane-3-carboxamide (Example 101)

2-(methylsulfonyl)pyridin-4-amine 101c (14 mg, 0.082 mmol, 1.5 eq) was dissolved in anhydrous pyridine (5 mL), and 4-(3,4-difluoro-2-methoxyphenyl)-5-methyl-1-trifluoromethyl-2-oxabicyclo[3.2.0]heptane-3-carboxylic acid 85d (20 mg, 0.055 mmol, 1.0 eq) and phosphorus oxychloride (25 mg, 0.16 mmol, 2.9 eq) were added. The mixture was stirred at 25° C. for 5 min. The filtrate was concentrated under reduced pressure, and purified by preparative separation (0.1% NH₃), to obtain the compound 101 (3.4 mg, 9% yield), which was a white solid. LCMS: [M+H]⁺=521.1. ¹H NMR (400 MHz, CD₃OD) δ 8.62 (s, 1H), 8.48 (s, 1H), 8.01-7.99 (m, 1H), 7.10-7.06 (m, 1H), 7.03-6.96 (m, 1H), 5.13-5.11 (m, 1H), 4.34 (d, J=6.4 Hz, 1H), 3.98 (d, J=2.0 Hz, 3H), 3.21 (s, 3H), 2.51-2.45 (m, 1H), 2.38-2.31 (m, 1H), 2.23-2.17 (m, 1H), 2.03-2.00 (m, 1H), 1.93-1.85 (m, 1H), 0.91-0.88 (m, 3H).

### Example 102: Synthesis of Compound 102

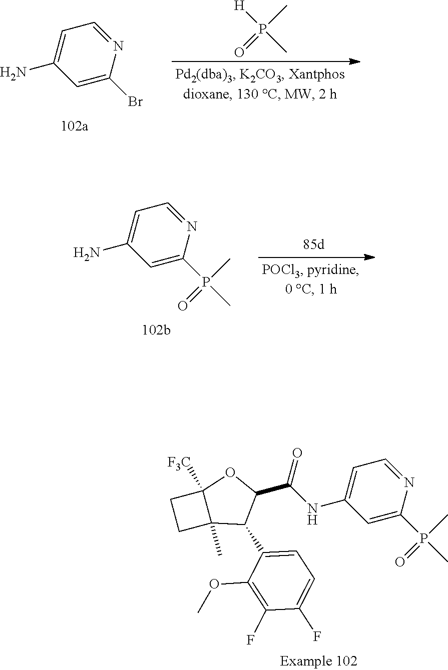

### Step 1: (4-aminopyridin-2-yl)dimethylphosphine oxide (102b)

In a microwave tube, the compound 2-bromopyridin-4-amine 102a (200 mg, 1.16 mmol, 1.0 eq) was dissolved in 1,4-dioxane (3 mL), and the compound dimethylphosphine oxide 2 (180 mg, 2.31 mmol, 2.0 eq), 4,5-bis(diphenylphosphino)-9,9-dimethylxanthene (67 mg, 0.12 mmol, 0.1 eq), tris(dibenzylideneacetone)palladium (106 mg, 0.12 mmol, 0.1 eq), and potassium carbonate (320 mg, 2.3 mmol, 2.0 eq) were added. The reaction solution underwent a microwave reaction at 130° C. in a nitrogen atmosphere for 2 h. After the completion of the reaction of the raw material was monitored by LCMS, the reaction solution was concentrated. The residue was purified by silica gel column chromatography (petroleum ether/ethyl acetate=8/1) to obtain the compound (4-aminopyridin-2-yl)dimethylphosphine oxide 102b (150 mg, 76% yield), which was a yellow solid.

### Step 2: rel-(1R,3R,4S,5R)-4-(3,4-difluoro-2-methoxyphenyl)-N-(2-(dimethylphosphoryl)pyridin-4-yl)-5-methyl-1-trifluoromethyl-2-oxabicyclo[3.2.0]heptane-3-carboxamide (102)

85d (50 mg, 0.14 mmol, 1.0 eq) was dissolved in anhydrous pyridine (3 mL), and the compound (4-aminopyridin-2-yl)dimethylphosphine oxide 102b (23 mg, 0.14 mmol, 1.0 eq) and phosphorus oxychloride (84 mg, 0.55 mmol, 4.0 eq) were added. The reaction solution reacted at 0° C. for 1 h. After the completion of the reaction of the raw material was monitored by LCMS, the reaction mixture was diluted with water (10 mL), subjected to extraction with ethyl acetate (10 mL×3), washed with water and saturated saline sequentially, dried over anhydrous sodium sulfate, and then concentrated. The residue was purified by preparative separation (0.05% FA) to obtain the compound 102 (15.8 mg, 22% yield), which was a white solid. LCMS: [M+H]⁺=519.1. ¹H NMR (400 MHz, DMSO-d₆) δ 10.77 (s, 1H), 8.63 (d, J=5.6 Hz, 1H), 8.28 (dd, J=6.8, 2.0 Hz, 1H), 7.85-7.82 (m, 1H), 7.23-7.16 (m, 1H), 7.13-7.07 (m, 1H), 5.13 (d, J=8.0 Hz, 1H), 4.23 (d, J=8.0 Hz, 1H), 3.93 (d, J=2.0 Hz, 3H), 2.44-2.35 (m, 2H), 2.19-2.12 (m, 1H), 1.87-1.80 (m, 1H), 1.64 (s, 3H), 1.60 (s, 3H), 0.82 (s, 3H).

### Example 103: Synthesis of Compound 103

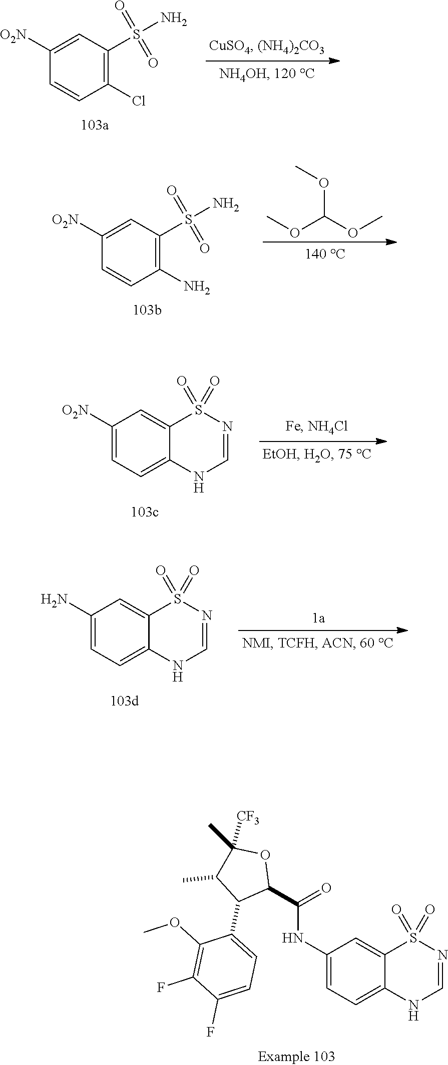

### Step 1: 2-amino-5-nitrobenzenesulfonamide (103b)

103a (2.0 g, 8.45 mmol, 1.0 eq), ammonium carbonate (1.62 g, 16.9 mmol, 2.0 eq), copper sulfate (405 mg, 2.54 mmol, 0.3 eq), and aqueous ammonia (10 mL) were added in a reaction flask and stirred at 120° C. for 6 h. The reaction was monitored by TLC until the raw material was fully converted. The reaction solution was cooled down to room temperature and poured into water to precipitate a solid. The mixture was filtered. The filter cake was washed with water and dried to obtain the compound 103b (1.3 g, 71% yield), which was a light yellow solid. LCMS: 218.1 [M+H]⁺.

### Step 2: 7-nitro-4H-benzol[e][1,2,4]thiadiazine-1,1-dioxide (103c)

103b (600 mg, 2.76 mmol, 1.0 eq) and trimethyl orthoformate (10 mL) were added in a reaction flask and stirred at 140° C. for 8 h. The reaction was monitored by TLC until the raw material was fully converted. The reaction solution was cooled down to room temperature and poured into water to precipitate a solid. The mixture was filtered. The filter cake was washed with water and dried to obtain the compound 103c (230 mg, 37% yield), which was a pale white solid. LCMS: 228.1 [M+H]⁺.

### Step 3: 7-amino-4H-benzo[e][1,2,4]thiadiazine-1,1-dioxide (103d)

103c (200 mg, 0.88 mmol, 1.0 eq), iron powder (246 mg, 4.4 mmol, 5.0 eq), ammonium chloride (141 mg, 2.64 mmol, 3.0 eq), ethanol (5 mL), and water (2 mL) were added in a reaction flask and stirred at 75° C. for 2 h. The reaction was monitored by LCMS until the raw material was fully converted. The reaction solution was filtered with diatomaceous earth while hot. The filtrate was concentrated and separated by column chromatography (MeOH/DCM, 0-10%), to obtain the compound 103d (82 mg, 47% yield), which was a light yellow solid. LCMS: 198.2 [M+H]⁺.

### Step 4: rel-(2R,3S,4S,5R)-3-(3,4-difluoro-2-methoxyphenyl)-N-(1,1-dihydroxy-4H-benzol[e][1,2,4]thiadiazine-7-yl)-4,5-dimethyl-5-(trifluoromethyl)tetrahydrofuran-2-carboxamide (Example 103)

1a (144 mg, 0.406 mmol, 1.0 eq), acetonitrile (3 mL), NMI (167 mg, 2.03 mmol, 5.0 eq), and TCFH (228 mg, 0.812 mmol, 2.0 eq) were added in a reaction flask and stirred for 5 min. Then, 103d (80 mg, 0.406 mmol, 1.0 eq) was added and stirred at 60° C. for 12 h. The reaction was monitored by LCMS until the raw material was fully converted. The reaction solution with water (10 mL) added was subjected to extraction with ethyl acetate (10 mL×3). The organic phases were combined, dried over anhydrous sodium sulfate, filtered, concentrated, and separated by column chromatography (MeOH/DCM, 0-10%), to obtain Example 103 (63 mg, 29% yield), which was a white solid. LCMS: 534.4 [M+H]⁺. ¹H NMR (400 MHz, dmso-d6) δ 12.28 (s, 1H), 10.54 (s, 1H), 8.22 (d, J=2.2 Hz, 1H), 7.93 (d, J=2.3 Hz, 1H), 7.82 (dd, J=9.0, 2.3 Hz, 1H), 7.30 (d, J=9.0 Hz, 1H), 7.24-7.07 (m, 2H), 5.07 (d, J=10.3 Hz, 1H), 4.25 (dd, J=10.2, 7.7 Hz, 1H), 3.95 (d, J=1.9 Hz, 3H), 2.85-2.70 (m, 1H), 1.61 (s, 3H), 0.72 (d, J=6.2 Hz, 3H).

### Example 104: Synthesis of Compound 104

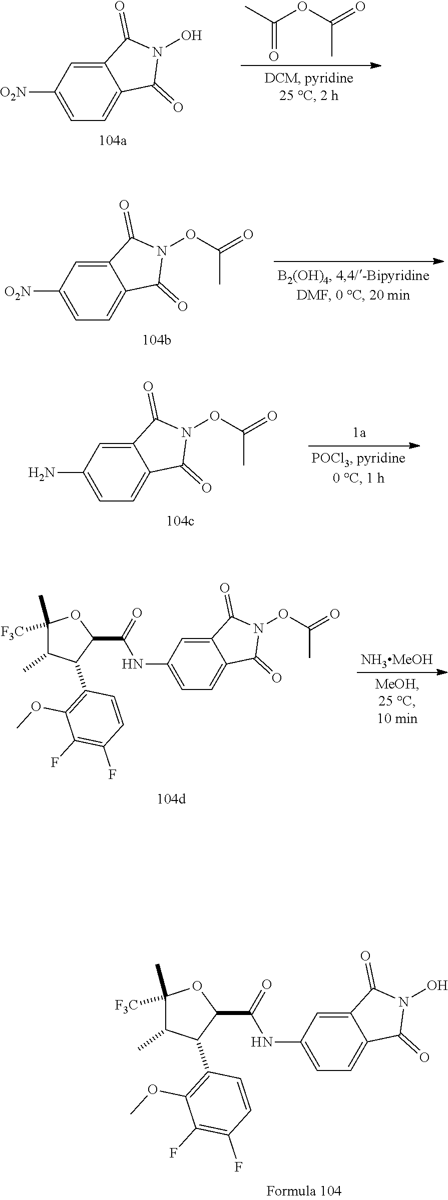

### Step 1: 5-nitro-1,3-dioxoisoindolin-2-yl acetate (104b)

In a single-necked flask, the compound 2-hydroxy-5-nitroisoindolin-1,3-dione 104a (300 mg, 1.44 mmol, 1.0 eq) was dissolved in dichloromethane (5 mL), and the compound acetic anhydride (221 mg, 2.17 mmol, 1.5 eq) and pyridine (228 mg, 2.88 mmol, 2.0 eq) were added. The reaction solution was stirred at 0° C. for 2 h. After the completion of the reaction of the raw material was monitored by LCMS, the reaction solution was concentrated. The residue was purified by silica gel column chromatography (petroleum ether/ethyl acetate=4/1) to obtain the compound 104b (300 mg, 83% yield), which was a white solid.

### Step 2: 5-amino-1,3-dioxoisoindolin-2-yl acetate (104c)

In a single-necked flask, the compound 104b (300 mg, 1.20 mmol, 1.0 eq) was dissolved in N,N-dimethylformamide (5 mL), and tetrahydroxydiboron (430 mg, 4.80 mmol, 4.0 eq) and 4,4′-dipyridyl (19 mg, 0.12 mmol, 0.1 eq) were added. The reaction solution reacted at 0° C. for 20 min. After the completion of the reaction of most of the raw materials was monitored by LCMS, ethyl acetate (20 mL) and water (20 mL) were added sequentially to the reaction mixture. The organic phase was washed once with saturated saline (20 mL), dried over anhydrous sodium sulfate, and filtered. The filtrate was concentrated. The residue was separated and purified by column chromatography (petroleum ether/ethyl acetate=1/1) to obtain the compound 104c (80 mg, 30% yield), which was a yellow oily substance.

### Step 3: rel-5-(2R,3S,4S,5R)-3-(3,4-difluoro-2-methoxyphenyl)-4,5-dimethyl-5-trifluoromethyl tetrahydrofuran-2-carboxamido)-1,3-dioxoisoindolin-2-acetate (104d)

The compound 1a (100 mg, 0.28 mmol, 1.0 eq) was dissolved in anhydrous pyridine (3 mL), and the compound 104c (62 mg, 0.28 mmol, 1.0 eq) and phosphorus oxychloride (173 mg, 1.13 mmol, 4.0 eq) were added. The reaction solution reacted at 0° C. for 1 h. After the completion of the reaction of the raw material was monitored by LCMS, the reaction mixture was diluted with water (10 mL), subjected to extraction with ethyl acetate (10 mL×3), washed with saturated saline, and filtered. The filtrate was concentrated. The residue was separated and purified by column chromatography (petroleum ether/ethyl acetate=4/1) to obtain the compound 104d (100 mg, 64% yield), which was a white solid.

### Step 4: rel-(2R,3S,4S,5R)-3-(3,4-difluoro-2-methoxyphenyl)-N-(2-hydroxy-1,3-dioxoisoindolin-5-yl)-4,5-dimethyl-5-trifluoromethyl tetrahydrofuran-2-carboxamide (Example 104)

The compound 104d (100 mg, 0.18 mmol, 1.0 eq) was dissolved in methanol (3 mL), and an ammonia in methanol solution (0.5 mL) was added. The reaction solution reacted at 25° C. for 10 min. After the completion of the reaction of the raw material was monitored by LCMS, the reaction mixture was concentrated. The residue was purified by preparative separation (0.1% FA) to obtain Example 104 (28.2 mg, 29% yield), which was a white solid. LCMS: [M−H]⁻=513.1.

¹H NMR (400 MHz, DMSO-d₆) δ 10.77 (s, 1H), 8.14 (d, J=1.6 Hz, 1H), 7.94 (dd, J=8.0, 2.0 Hz, 1H), 7.78 (d, J=8.0 Hz, 1H), 7.21-7.14 (m, 2H), 5.12 (d, J=10.0 Hz, 1H), 4.29-4.25 (m, 1H), 3.95 (d, J=2.0 Hz, 3H), 2.82-2.74 (m, 1H), 1.61 (s, 3H), 0.74 (d, J=5.6 Hz, 3H).

TABLE 1
The following compounds were prepared with corresponding reagents as raw
materials according to operations similar to the operations described in the foregoing examples.

| | |
|---|---|
| 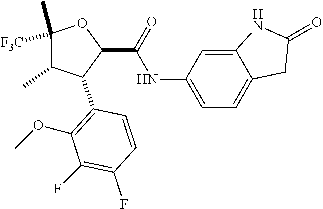 | 2 |
| ESI-MS m/z: 485.1 (M + 1)⁺ | |
| 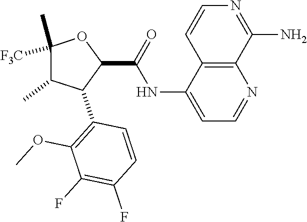 | 7 |
| ESI-MS m/z: 497.2 (M + 1)⁺ | |
| 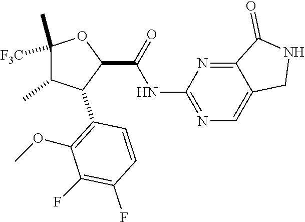 | 11 |
| ESI-MS m/z: 487.1 (M + 1)⁺ | |
| 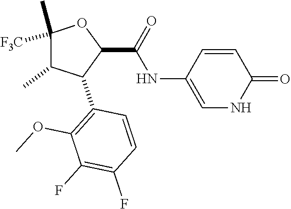 | 14 |
| ESI-MS m/z: 447.1 (M + 1)⁺ | |
| 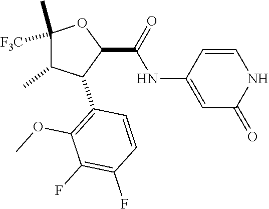 | 15 |
| ESI-MS m/z: 447.1 (M + 1)⁺ | |
| 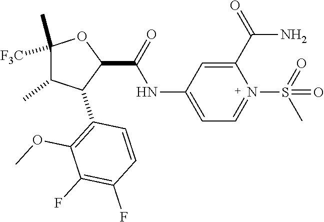 | 17 |
| ESI-MS m/z: 553.1 (M + 1)⁺ | |
| 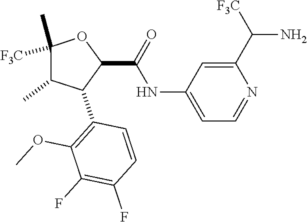 | 18 |
| ESI-MS m/z: 528.2 (M + 1)⁺ | |
| 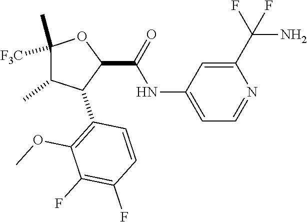 | 19 |
| ESI-MS m/z: 496.1 (M + 1)⁺ | |
| 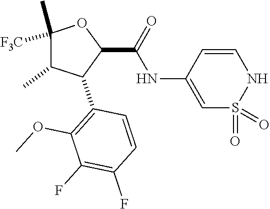 | 23 |
| ESI-MS m/z: 483.1 (M + 1)⁺ | |
| 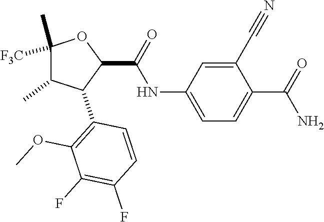 | 26 |
| ESI-MS m/z: 498.1 (M + 1)⁺ | |
| 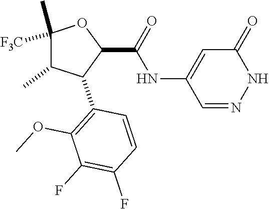 | 28 |
| ESI-MS m/z: 448.1 (M + 1)⁺ | |
| 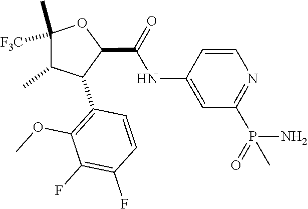 | 33 |
| ESI-MS m/z: 508.1 (M + 1)⁺ | |
| 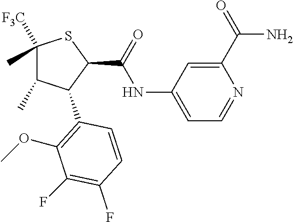 | 35 |
| ESI-MS m/z: 490.1 (M + 1)⁺ | |
| 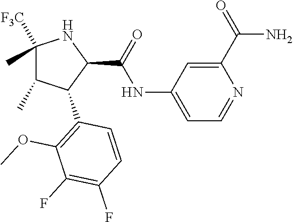 | 36 |
| ESI-MS m/z: 473.2 (M + 1)⁺ | |
| 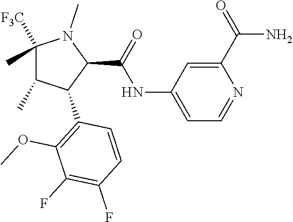 | 37 |
| ESI-MS m/z: 487.2 (M + 1)⁺ | |
| 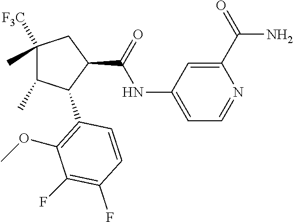 | 38 |
| ESI-MS m/z: 472.2 (M + 1)⁺ | |
| 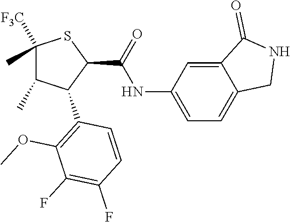 | 39 |
| ESI-MS m/z: 501.1 (M + 1)⁺ | |
| 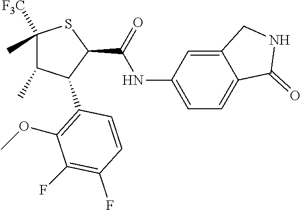 | 40 |
| ESI-MS m/z: 501.1 (M + 1)⁺ | |
| 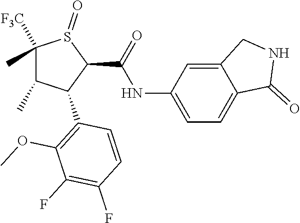 [SMILES: COc1c([C@H]2[C@H](C(=O)Nc3ccc4c(c3)CNC4=O)S(=O)[C@@](C)(C(F)(F)F)[C@H]2C)ccc(F)c1F] | 41 |
| ESI-MS m/z: 517.1 (M + 1)⁺ | |
| 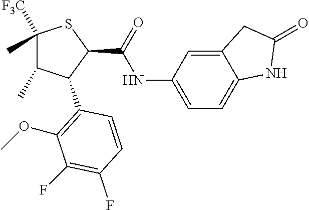 | 42 |
| ESI-MS m/z: 501.1 (M + 1)⁺ | |
| 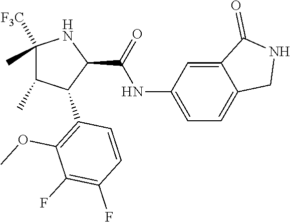 | 43 |
| ESI-MS m/z: 484.2 (M + 1)⁺ | |
| 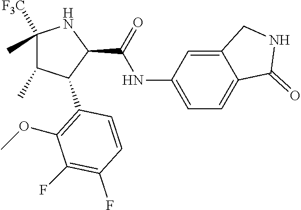 | 44 |
| ESI-MS m/z: 484.2 (M + 1)⁺ | |
| 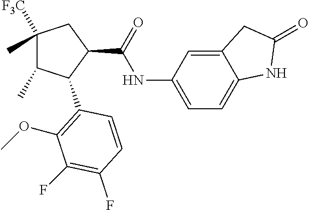 | 45 |
| ESI-MS m/z: 483.2 (M + 1)⁺ | |
| 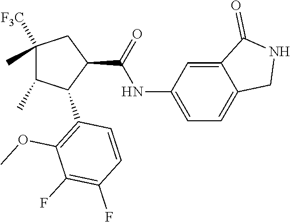 | 46 |
| ESI-MS m/z: 483.2 (M + 1)⁺ | |
| 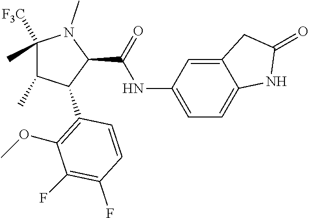 | 47 |
| ESI-MS m/z: 498.2 (M + 1)⁺ | |
| 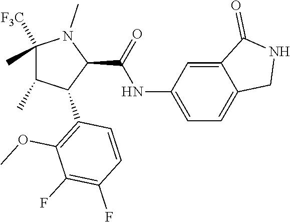 | 48 |
| ESI-MS m/z: 498.2 (M + 1)⁺ | |
| 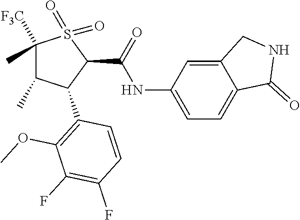 | 49 |
| ESI-MS m/z: 533.1 (M + 1)⁺ | |
| 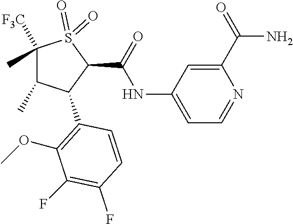 | 50 |
| ESI-MS m/z: 522.1 (M + 1)⁺ | |
| 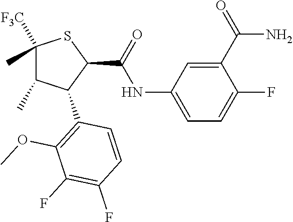 [SMILES: COc1c([C@H]2[C@H](C(=O)Nc3ccc(F)c(C(N)=O)c3)S[C@@](C)(C(F)(F)F)[C@H]2C)ccc(F)c1F] | 51 |
| ESI-MS m/z: 507.1 (M + 1)⁺ | |
| 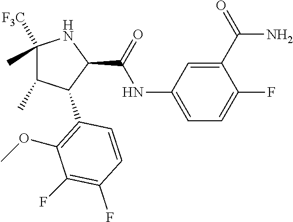 | 52 |
| ESI-MS m/z: 490.2 (M + 1)⁺ | |
| 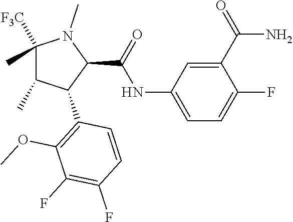 | 53 |
| ESI-MS m/z: 504.2 (M + 1)⁺ | |
| 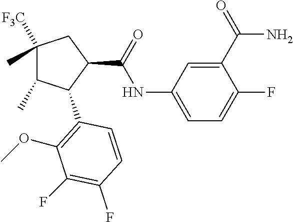 | 54 |
| ESI-MS m/z: 489.2 (M + 1)⁺ | |
| 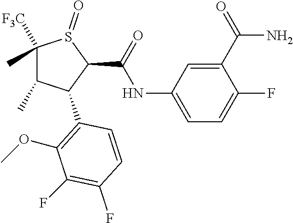 | 55 |
| ESI-MS m/z: 523.1 (M + 1)⁺ | |
| 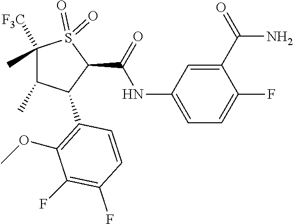 | 56 |
| ESI-MS m/z: 539.1 (M + 1)⁺ | |
| 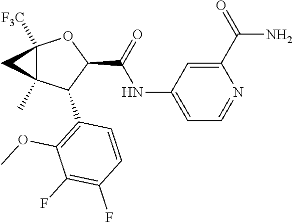 [SMILES: COc1c([C@H]2[C@H](C(=O)Nc3ccnc(C(N)=O)c3)O[C@]3(C(F)(F)F)C[C@]23C)ccc(F)c1F] | 57 |
| ESI-MS m/z: 472.1 (M + 1)⁺ | |
| 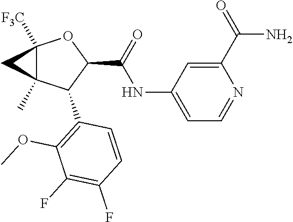 [SMILES: COc1c([C@H]2[C@H](C(=O)Nc3ccnc(C(N)=O)c3)O[C@]3(C(F)(F)F)C[C@]23C)ccc(F)c1F] | 58 |
| ESI-MS m/z: 472.1 (M + 1)⁺ | |
| 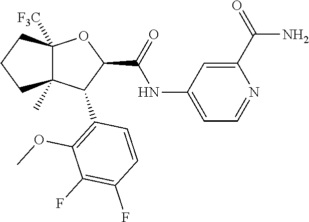 [SMILES: COc1c([C@H]2[C@H](C(=O)Nc3ccnc(C(N)=O)c3)O[C@]3(C(F)(F)F)CCC[C@]23C)ccc(F)c1F] | 60 |
| ESI-MS m/z: 500.2 (M + 1)⁺ | |
| 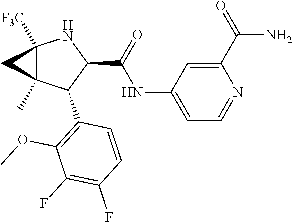 [SMILES: COc1c([C@H]2[C@H](C(=O)Nc3ccnc(C(N)=O)c3)N[C@]3(C(F)(F)F)C[C@]23C)ccc(F)c1F] | 61 |
| ESI-MS m/z: 471.1 (M + 1)⁺ | |
| 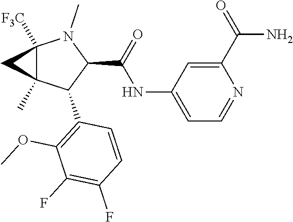 [SMILES: COc1c([C@H]2[C@H](C(=O)Nc3ccnc(C(N)=O)c3)N(C)[C@]3(C(F)(F)F)C[C@]23C)ccc(F)c1F] | 62 |
| ESI-MS m/z: 485.2 (M + 1)⁺ | |
| 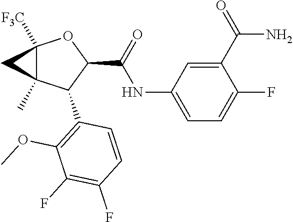 [SMILES: COc1c([C@H]2[C@H](C(=O)Nc3ccc(F)c(C(N)=O)c3)O[C@]3(C(F)(F)F)C[C@]23C)ccc(F)c1F] | 63 |
| ESI-MS m/z: 489.1 (M + 1)⁺ | |
| 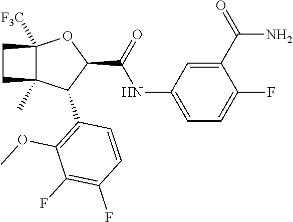 [SMILES: COc1c([C@H]2[C@H](C(=O)Nc3ccc(F)c(C(N)=O)c3)O[C@]3(C(F)(F)F)CC[C@]23C)ccc(F)c1F] | 64 |
| ESI-MS m/z: 503.1 (M + 1)⁺ | |
| 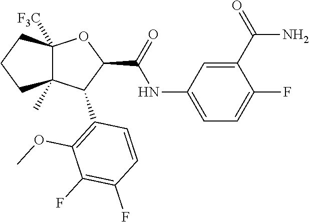 [SMILES: COc1c([C@H]2[C@H](C(=O)Nc3ccc(F)c(C(N)=O)c3)O[C@]3(C(F)(F)F)CCC[C@]23C)ccc(F)c1F] | 65 |
| ESI-MS m/z: 517.2 (M + 1)⁺ | |
| 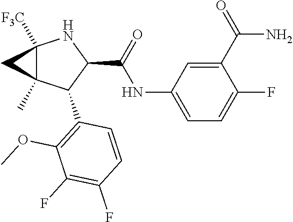 [SMILES: COc1c([C@H]2[C@H](C(=O)Nc3ccc(F)c(C(N)=O)c3)N[C@]3(C(F)(F)F)C[C@]23C)ccc(F)c1F] | 66 |
| ESI-MS m/z: 488.1 (M + 1)⁺ | |
| 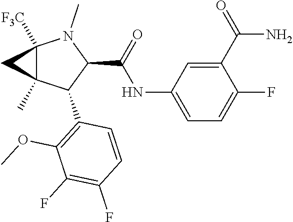 [SMILES: COc1c([C@H]2[C@H](C(=O)Nc3ccc(F)c(C(N)=O)c3)N(C)[C@]3(C(F)(F)F)C[C@]23C)ccc(F)c1F] | 67 |
| ESI-MS m/z: 502.2 (M + 1)⁺ | |
| 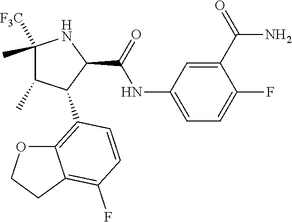 [SMILES: C[C@H]1[C@@H](c2ccc(F)c3c2OCC3)[C@H](C(=O)Nc2ccc(F)c(C(N)=O)c2)N[C@@]1(C)C(F)(F)F] | 68 |
| ESI-MS m/z: 484.2 (M + 1)⁺ | |
| 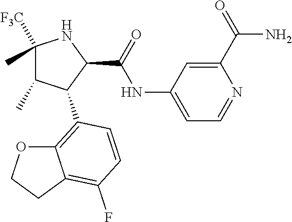 [SMILES: C[C@H]1[C@@H](c2ccc(F)c3c2OCC3)[C@H](C(=O)Nc2ccnc(C(N)=O)c2)N[C@@]1(C)C(F)(F)F] | 69 |
| ESI-MS m/z: 467.2 (M + 1)⁺ | |
| 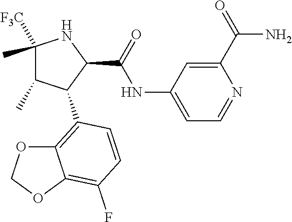 [SMILES: C[C@H]1[C@@H](c2ccc(F)c3c2OCO3)[C@H](C(=O)Nc2ccnc(C(N)=O)c2)N[C@@]1(C)C(F)(F)F] | 70 |
| ESI-MS m/z: 469.1 (M + 1)⁺ | |
| 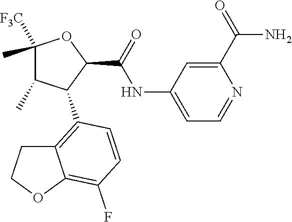 [SMILES: C[C@H]1[C@@H](c2ccc(F)c3c2CCO3)[C@H](C(=O)Nc2ccnc(C(N)=O)c2)O[C@@]1(C)C(F)(F)F] | 71 |
| ESI-MS m/z: 467.1 (M + 1)⁺ | |
| 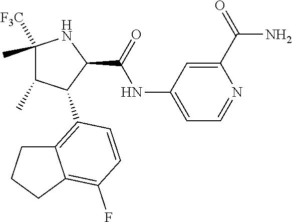 [SMILES: C[C@H]1[C@@H](c2ccc(F)c3c2CCC3)[C@H](C(=O)Nc2ccnc(C(N)=O)c2)N[C@@]1(C)C(F)(F)F] | 72 |
| ESI-MS m/z: 465.2 (M + 1)⁺ | |
| 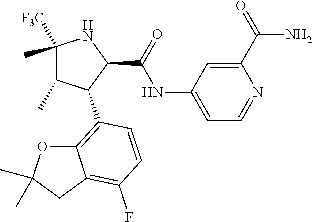 [SMILES: C[C@H]1[C@@H](c2ccc(F)c3c2OC(C)(C)C3)[C@H](C(=O)Nc2ccnc(C(N)=O)c2)N[C@@]1(C)C(F)(F)F] | 73 |
| ESI-MS m/z: 495.2 (M + 1)⁺ | |
| 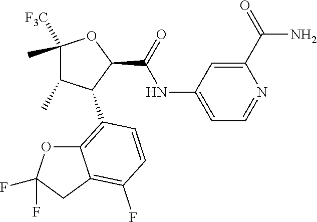 [SMILES: C[C@H]1[C@@H](c2ccc(F)c3c2OC(F)(F)C3)[C@H](C(=O)Nc2ccnc(C(N)=O)c2)O[C@@]1(C)C(F)(F)F] | 74 |
| ESI-MS m/z: 503.1 (M + 1)⁺ | |
| 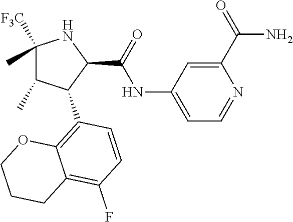 [SMILES: C[C@H]1[C@@H](c2ccc(F)c3c2OCCC3)[C@H](C(=O)Nc2ccnc(C(N)=O)c2)N[C@@]1(C)C(F)(F)F] | 75 |
| ESI-MS m/z: 481.2 (M + 1)⁺ | |
| 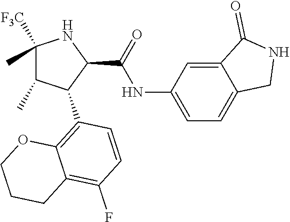 [SMILES: C[C@H]1[C@@H](c2ccc(F)c3c2OCCC3)[C@H](C(=O)Nc2ccc3c(c2)C(=O)NC3)N[C@@]1(C)C(F)(F)F] | 76 |
| ESI-MS m/z: 492.2 (M + 1)⁺ | |
| 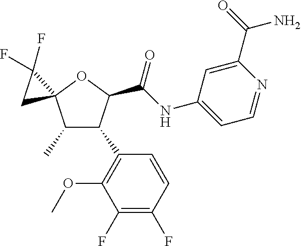 [SMILES: COc1c([C@H]2[C@H](C(=O)Nc3ccnc(C(N)=O)c3)O[C@]3(CC3(F)F)[C@H]2C)ccc(F)c1F] | 77 |
| ESI-MS m/z: 454.1 (M + 1)⁺ | |
| 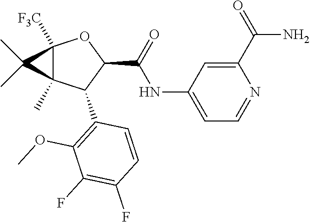 [SMILES: COc1c([C@H]2[C@H](C(=O)Nc3ccnc(C(N)=O)c3)O[C@]3(C(F)(F)F)C(C)(C)[C@]23C)ccc(F)c1F] | 78 |
| ESI-MS m/z: 500.2 (M + 1)⁺ | |
| 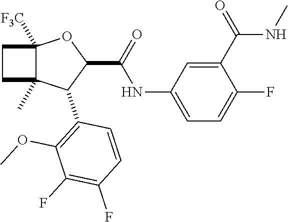 | 105 |
| ESI-MS m/z: 517.2 (M + 1)⁺ | |
| 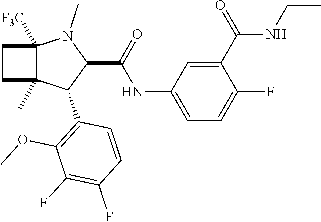 | 106 |
| ESI-MS m/z: 531.2 (M + 1)⁺ | |
| 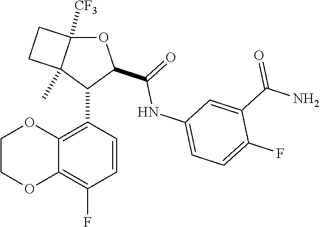 | 107 |
| ESI-MS m/z: 513.1 (M + 1)⁺ | |
| 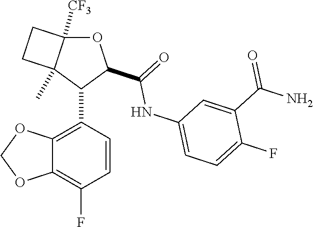 | 108 |
| ESI-MS m/z: 499.1 (M + 1)⁺ | |
| 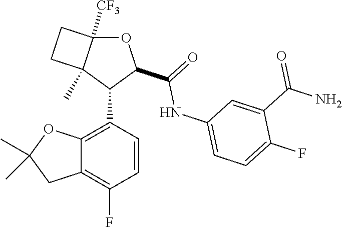 [SMILES: CC1(C)Cc2c(F)ccc([C@H]3[C@H](C(=O)Nc4ccc(F)c(C(N)=O)c4)O[C@]4(C(F)(F)F)CC[C@]34C)c2O1] | 109 |
| ESI-MS m/z: 525.2 (M + 1)⁺ | |
| 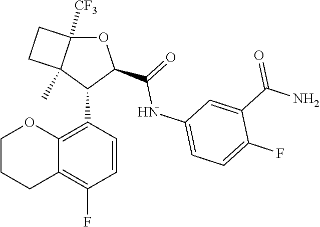 [SMILES: C[C@]12CC[C@@]1(C(F)(F)F)O[C@@H](C(=O)Nc1ccc(F)c(C(N)=O)c1)[C@@H]2c1ccc(F)c2c1OCCC2] | 110 |
| ESI-MS m/z: 511.2 (M + 1)⁺ | |
| 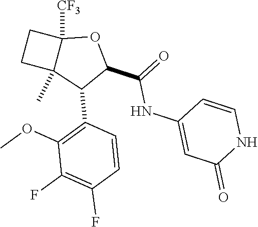 | 111 |
| ESI-MS m/z: 459.1 (M + 1)⁺ | |
| 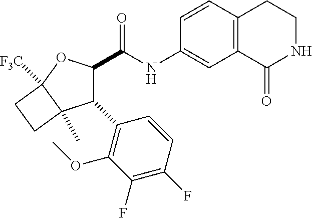 [SMILES: COc1c([C@H]2[C@H](C(=O)Nc3ccc4c(c3)C(=O)NCC4)O[C@]3(C(F)(F)F)CC[C@]23C)ccc(F)c1F] | 112 |
| ESI-MS m/z: 511.2 (M + 1)⁺ | |
| 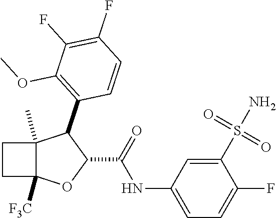 [SMILES: COc1c([C@H]2[C@H](C(=O)Nc3ccc(F)c(S(N)(=O)=O)c3)O[C@]3(C(F)(F)F)CC[C@@]23C)ccc(F)c1F] | 113 |
| ESI-MS m/z: 539.1 (M + 1)⁺ | |
| 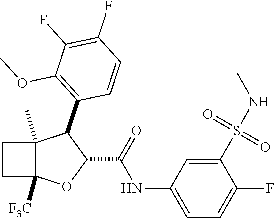 [SMILES: CNS(=O)(=O)c1cc(NC(=O)[C@@H]2O[C@]3(C(F)(F)F)CC[C@@]3(C)[C@H]2c2ccc(F)c(F)c2OC)ccc1F] | 114 |
| ESI-MS m/z: 553.1 (M + 1)⁺ | |
| 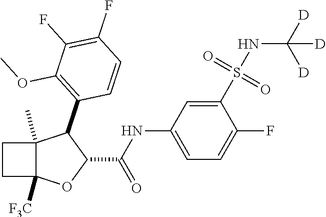 [SMILES: [2H]C([2H])([2H])NS(=O)(=O)c1cc(NC(=O)[C@@H]2O[C@]3(C(F)(F)F)CC[C@@]3(C)[C@H]2c2ccc(F)c(F)c2OC)ccc1F] | 115 |
| ESI-MS m/z: 556.1 (M + 1)⁺ | |
| 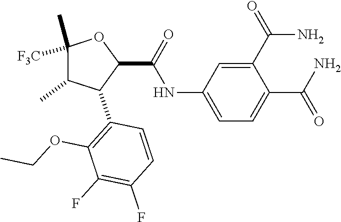 | 116 |
| ESI-MS m/z: 530.2 (M + 1)⁺ | |
| 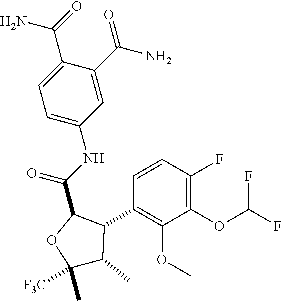 | 117 |
| ESI-MS m/z: 564.2 (M + 1)⁺ | |
| 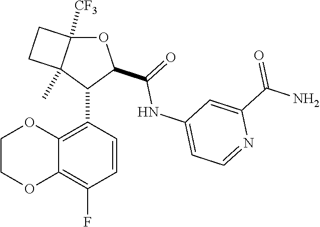 | 118 |
| ESI-MS m/z: 496.1 (M + 1)⁺ | |
| 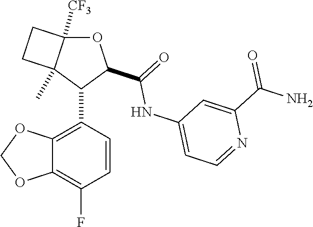 | 119 |
| ESI-MS m/z: 482.1 (M + 1)⁺ | |
| 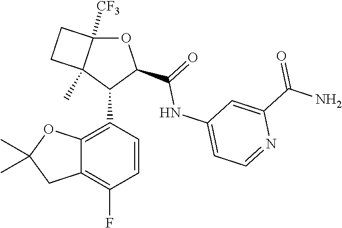 [SMILES: CC1(C)Cc2c(F)ccc([C@H]3[C@H](C(=O)Nc4ccnc(C(N)=O)c4)O[C@]4(C(F)(F)F)CC[C@]34C)c2O1] | 120 |
| ESI-MS m/z: 508.2 (M + 1)⁺ | |
| 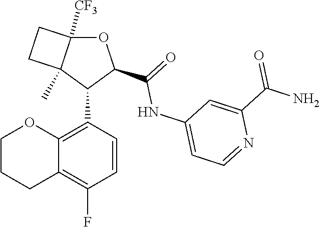 [SMILES: C[C@]12CC[C@@]1(C(F)(F)F)O[C@@H](C(=O)Nc1ccnc(C(N)=O)c1)[C@@H]2c1ccc(F)c2c1OCCC2] | 121 |
| ESI-MS m/z: 494.2 (M + 1)⁺ | |
| 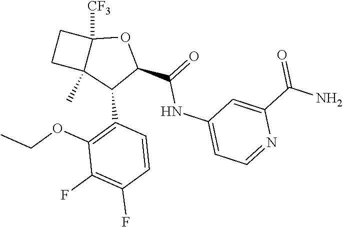 | 122 |
| ESI-MS m/z: 500.2 (M + 1)⁺ | |
| 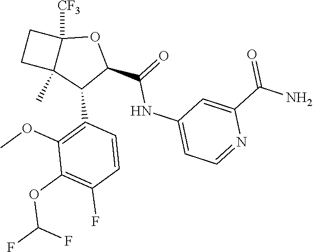 | 123 |
| ESI-MS m/z: 534.1 (M + 1)⁺ | |
| 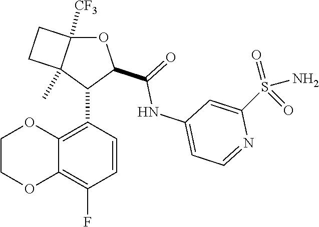 | 124 |
| ESI-MS m/z: 532.1 (M + 1)⁺ | |
| 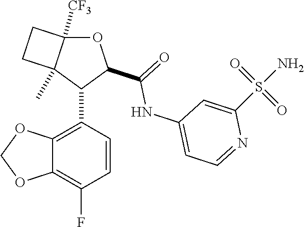 | 125 |
| ESI-MS m/z: 518.1 (M + 1)⁺ | |
| 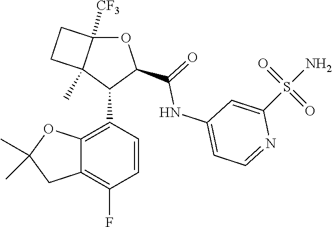 [SMILES: CC1(C)Cc2c(F)ccc([C@H]3[C@H](C(=O)Nc4ccnc(S(N)(=O)=O)c4)O[C@]4(C(F)(F)F)CC[C@]34C)c2O1] | 126 |
| ESI-MS m/z: 544.2 (M + 1)⁺ | |
| 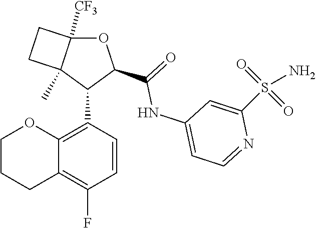 [SMILES: C[C@]12CC[C@@]1(C(F)(F)F)O[C@@H](C(=O)Nc1ccnc(S(N)(=O)=O)c1)[C@@H]2c1ccc(F)c2c1OCCC2] | 127 |
| ESI-MS m/z: 530.1 (M + 1)⁺ | |
| 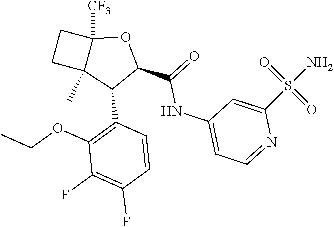 | 128 |
| ESI-MS m/z: 536.1 (M + 1)⁺ | |
| 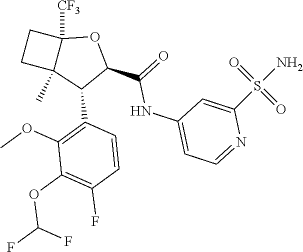 | 129 |
| ESI-MS m/z: 570.1 (M + 1)⁺ | |
| 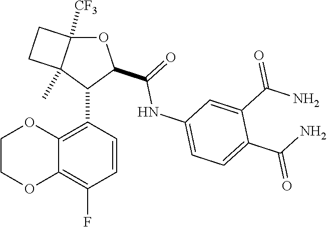 | 130 |
| ESI-MS m/z: 538.2 (M + 1)⁺ | |
| 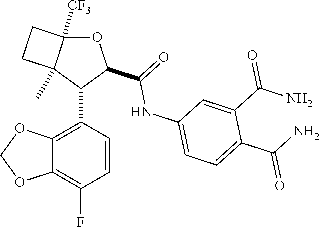 | 131 |
| ESI-MS m/z: 524.1 (M + 1)⁺ | |
| 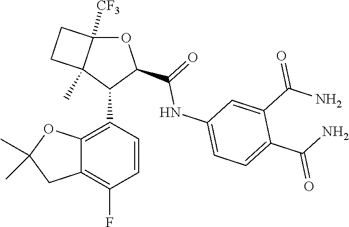 [SMILES: CC1(C)Cc2c(F)ccc([C@H]3[C@H](C(=O)Nc4ccc(C(N)=O)c(C(N)=O)c4)O[C@]4(C(F)(F)F)CC[C@]34C)c2O1] | 132 |
| ESI-MS m/z: 550.2 (M + 1)⁺ | |
| 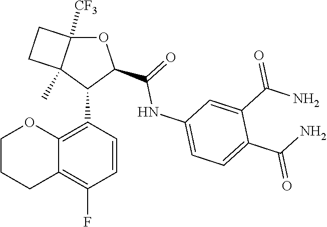 [SMILES: C[C@]12CC[C@@]1(C(F)(F)F)O[C@@H](C(=O)Nc1ccc(C(N)=O)c(C(N)=O)c1)[C@@H]2c1ccc(F)c2c1OCCC2] | 133 |
| ESI-MS m/z: 536.2 (M + 1)⁺ | |
| 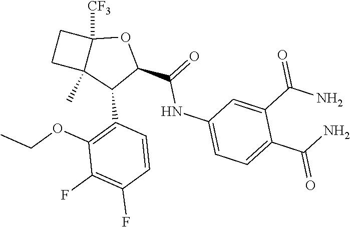 | 134 |
| ESI-MS m/z: 542.2 (M + 1)⁺ | |
| 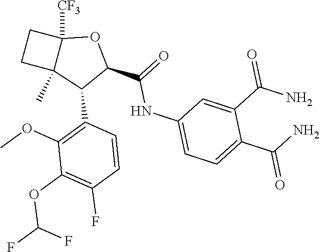 | 135 |
| ESI-MS m/z: 576.2 (M + 1)⁺ | |
| 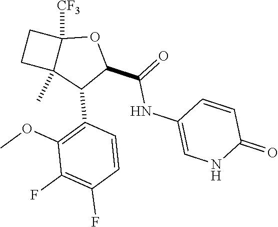 | 136 |
| ESI-MS m/z: 459.1 (M + 1)⁺ | |
| 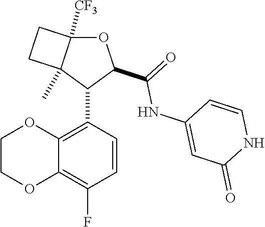 | 137 |
| ESI-MS m/z: 469.1 (M + 1)⁺ | |
| 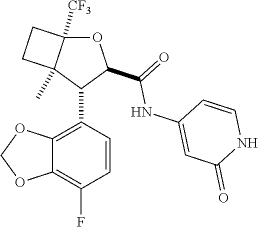 | 138 |
| ESI-MS m/z: 455.1 (M + 1)⁺ | |
| 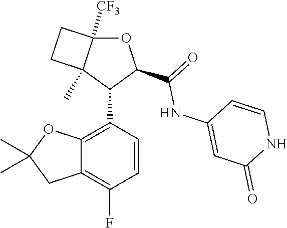 [SMILES: CC1(C)Cc2c(F)ccc([C@H]3[C@H](C(=O)Nc4cc[nH]c(=O)c4)O[C@]4(C(F)(F)F)CC[C@]34C)c2O1] | 139 |
| ESI-MS m/z: 481.2 (M + 1)⁺ | |
| 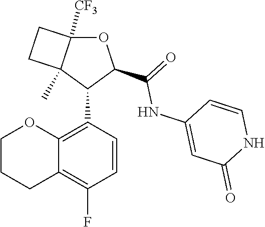 [SMILES: C[C@]12CC[C@@]1(C(F)(F)F)O[C@@H](C(=O)Nc1cc[nH]c(=O)c1)[C@@H]2c1ccc(F)c2c1OCCC2] | 140 |
| ESI-MS m/z: 467.2 (M + 1)⁺ | |
| 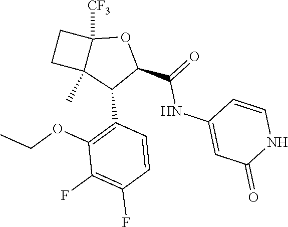 | 141 |
| ESI-MS m/z: 473.1 (M + 1)⁺ | |
| 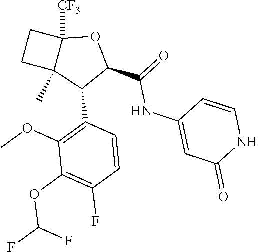 | 142 |
| ESI-MS m/z: 507.1 (M + 1)⁺ | |
| 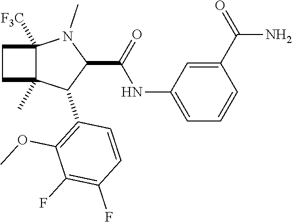 | 143 |
| ESI-MS m/z: 485.1 (M + 1)⁺ | |
| 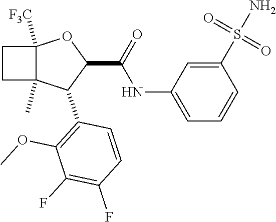 [SMILES: COc1c([C@H]2[C@H](C(=O)Nc3cccc(S(N)(=O)=O)c3)O[C@]3(C(F)(F)F)CC[C@]23C)ccc(F)c1F] | 144 |
| ESI-MS m/z: 521.1 (M + 1)⁺ | |

### Bioactivity Test 1: In Vitro Inhibition of Naᵥ1.8 by the Compound

By using a manual patch clamp on HEK293 cells stably expressing human Naᵥ1.8, currents on Naᵥ1.8 before and after administration of the compound were compared after the currents on Naᵥ1.8 were stable, to obtain the effect of the compound on the Naᵥ1.8 channel.

The to-be-tested compound was dissolved in DMSO to prepare a 9 mM stock solution, which was taken and dissolved in extracellular fluid at a desired concentration on the day of testing. The extracellular fluid included the following ingredients (mM): NaCl, 137; KCl, 4; CaCl₂, 1.8; MgCl₂, 1; HEPES, 10; glucose, 10; and pH 7.4 (NaOH titration). All reagents were purchased from Sigma (St. Louis, MO).

The cells were clamped at −80 mV and depolarized with a sustained 10 ms square wave to 10 mV to obtain a current on Naᵥ1.8. This process was repeated every 5 seconds. A maximum current caused by the square wave was detected. After the current is stable, the to-be-tested compound (dissolved in the extracellular fluid at the desired concentration) was perfused. After the current is stable, the strength of blocking Naᵥ1.8 by the compound was calculated based on the currents before and after perfusion of the compound. pCLAMP 10 (Molecular Devices, Union City, CA) was used. That the current is stable means that the current changes over time within a limited range.

TABLE 1
Inhibition determination (inhibition/% at 100 nM
concentration: A ≥ 50, B < 50)

| Example | Inhibitory activity |
|---|---|
| 1 | A |
| 2 | A |
| 3 | A |
| 4 | A |
| 5 | A |
| 6 | A |
| 7 | A |
| 8 | A |
| 9 | A |
| 10 | A |
| 12 | A |
| 13 | A |
| 16 | A |
| 21 | A |
| 22 | A |
| 24 | A |
| 25 | A |
| 27 | A |
| 30 | A |
| 30-1 | B |
| 30-2 | A |
| 32 | A |
| 59 | A |
| 79 | A |
| 80 | A |
| 81 | A |
| 82 | A |
| 83 | A |
| 84 | A |
| 85 | A |
| 86 | A |
| 87 | A |
| 88 | A |
| 89 | A |
| 92 | A |
| 93 | A |
| 94 | A |
| 95 | A |
| 96 | A |
| 97 | A |
| 98 | A |
| 99 | A |
| 100 | A |
| 101 | A |

The inhibition/% of Naᵥ1.8 at 100 nM concentration was determined for other compounds in the present invention by using the same method. It indicates that the compound of the present invention has excellent sodium channel-modulating activity (>50%), which can be used for the treatment of diseases related to sodium channel modulation.

Bioactivity Test 2: In Vitro Inhibition IC₅₀ of Naᵥ1.8 by the Test Compound

A CHO cell line stably expressing the Naᵥ1.8 sodium channel was used for experimental assays with the following gene information: Sodium channel, voltage-gated, type 8, alpha (SCN10A), cDNA strictly similar to GenBank accession number: NM_006514.

Before the patch clamp assay, the cells were detached with 0.25%-Trypsin-EDTA, and 6.5×10³ cells were spread onto cover slips and cultured in a 24-well plate (final volume: 500 μL) for 18 h. Then, the assay was carried out.

Fluids for electrophysiological recording included: extracellular fluid: K-007-1, 140 mM NaCl, 3.5 mM KCl, 1 mM MgCl₂·6H₂O, 2 mM CaCl₂·2H₂O, 10 mM D-Glucose, 10 mM HEPES, 1.25 mM NaH₂PO₄·2H₂O, and NaOH for adjustment to pH=7.4; and intracellular fluid: Nav-001-2, 50 mM CsCl, 10 mM NaCl, 10 mM HEPES, 60 mM CsF, 20 mM EGTA, and for adjustment to pH=7.2. The extracellular fluid was stored for 2 weeks. The intracellular fluid was prepared and dispensed as 1 mL per tube, and frozen in a refrigerator at −20° C. Freshly thawed intracellular fluid was used for daily experiments. All intracellular fluids have to be used up within three months. Beyond three months, the old intracellular fluid has to be discarded, and the intracellular fluid needs to be prepared again.

Patch clamp assay: Whole-cell patch-clamp recording Naᵥ1.8 sodium currents under voltage stimulation was carried out as follows: When a whole-cell seal was formed, the cell was clamped at a voltage of −120 mV and maintained for 30 ms. The cell was depolarized from the clamping voltage to 0 mV and maintained for 50 ms. Then, the voltage was restored to −50 mV (for a specific voltage, refer to a half-inactivation voltage from an IV test) and maintained for 5 s. Next, the cell membrane potential was restored to −120 mV and maintained for 20 ms. Then, the cell was depolarized to 0 mV and maintained for 50 ms. Finally, the voltage was restored to the clamping voltage −120 mV and maintained for 30 ms. Data was collected at 20 ms intervals. The effect of the drug on a peak sodium current was observed. Experimental data was collected by an EPC 10 amplifier (HEKA) and stored in PatchMaster (HEKA) software.

A capillary glass tube was pulled into a recording electrode by using a microelectrode puller. The electrode filled with the intracellular fluid was loaded into an electrode holder. A microelectrode manipulator was manipulated under an inverted microscope to immerse the electrode into the extracellular fluid, and an electrode resistance (Rpip) was recorded. The electrode was brought into contact with the cell surface, and suction was applied under negative pressure to form a high-resistance seal (GQ). In this case, fast capacitance compensation was carried out, and negative pressure was then applied to break the cell membrane through suction to form a whole-cell recording mode. Then, slow capacitance compensation was carried out, and experimental parameters such as a membrane capacitance (Cm) and a series resistance (Rs) were recorded. No leakage compensation was carried out.

The drug was administered after the Naᵥ1.8 current obtained by whole-cell recording. The drug acted at each concentration for 5 min (or until the current was stable), and then the drug was tested at a next concentration. Each test compound was tested for multiple concentrations. The cover slips with the cells spread were placed in a recording bath under an inverted microscope. A blank control extracellular fluid and a working solution of the to-be-tested compound were perfused by gravity through the recording bath into the cells from low concentration to high concentration. Fluid exchange was carried out by using a peristaltic pump during the recording. A current detected for each cell in the compound-free extracellular fluid served as its own control. For each concentration, at least three cells were tested independently for three times. All the electrophysiological tests were carried out at room temperature.

Data analysis: First, the current after the action of drug at each concentration and the blank control current were normalized

$$( \frac{Peak tail {current}_{compound}}{Peak tail {current}_{control}} ) .$$

Then, the inhibition

$$( 1 - \frac{Peak tail {current}_{compound}}{Peak tail {current}_{control}} )$$

corresonding to each drug concentration was calculated, and the mean (Mean), standard deviation (SD), and standard error (SE) were calculated for the inhibition of each concentration.

Y=Bottom+(Top−Bottom)/(1+10\hat{ }((Log IC₅₀−X)×HillSlope))

The IC₅₀ value of each compound was calculated by using the foregoing equation, and nonlinear fitting was carried out for the dose-dependent effect, where IC₅₀ is the half-maximal inhibitory concentration. The calculation of IC₅₀ and curve fitting were carried out using GraphPad Prism software.

| Example | IC₅₀ (nM) |
|---|---|
| 2 | 67 |
| 3 | 9 |
| 6 | 2 |
| 10 | 10 |
| 21 | 26 |
| 30-2 | <0.5 |
| 59 | 2 |
| 85 | <1 |
| 87 | 3 |
| 81 | <0.5 |
| 82 | <1 |

The inhibition of Naᵥ1.8 was tested for other compounds in the present invention by using the same method. It indicates that the compound of the present invention has a good inhibitory effect on Naᵥ1.8.

### Bioactivity Test 3: Efficacy of the Compound on a Mouse Model of Incisional Pain

Model establishment: Through isoflurane anesthesia and routine disinfection, a 0.5 cm longitudinal skin incision extending to the toe was made with a blade on the plantar surface of the left hindfoot about 0.2 cm distal to the joint; and the plantar muscle was separated, slightly elevated, and then incised longitudinally, taking care not to disrupt the muscle origin. The incision was sutured horizontally and interruptedly with 5-0 nylon thread (2 sutures).

Solvents: A solvent for the test compound was 5% DMSO+10% Solutol+85% Saline, and a solvent for a positive compound TRV-130 was 10% ethanol+10% cremophor+80% water.

A control compound 1 was the compound 7 in WO2021113627A1 synthesized according to the method in WO2021113627A1.

Mode and frequency of administration: The positive control compound TRV-130 was administered intravenously once daily; and oral administration was applied for the remaining groups once daily.

Experimental grouping: Before modeling, abnormal animals were excluded according to the PWT value. Then, 8 animals were randomly selected as a sham surgery group. 8 animals in each drug administration group were surgically modeled. The remaining animals were used as backup. After surgery, the animals were grouped again according to the pain threshold (PWT) before drug administration. If the animals were found to have abnormal PWT values after surgery, other animals were randomly selected from the backup animals and re-modeled.

Mechanical pain threshold measurement: Before modeling, a pre-modeling mechanical pain threshold was measured. At 2.5 h after modeling, a pre-administration mechanical pain threshold was measured. Oral administration was carried out after the measurement, and a mechanical pain threshold was measured at 2 h after the administration. The measurement was carried out at an inner side of the surgical incision. Each animal was measured three times at intervals of 3-5 minutes, and an average value was calculated.

Data analysis: Relevant data was organized in Office Excel 2013 and GraphPad Prism 6.0. Data was expressed as Mean±SEM (standard error). Analysis was carried out using One-way ANOVA by Tukey test for significance of differences between groups. A two-tailed test was carried out between two groups by T-test for comparison. When p<0.05, there is a significant difference between the two groups. When there are a few large differences in the test data within the groups, identify outliters data analysis may be carried out using GraphPad Prism 9.0 to exclude values with differences (Q=5%). During data statistics, all relevant data of the animals excluded from the pharmacodynamic experiment is excluded from the statistical analysis.

| | PWT (g) | |
|---|---|---|
| | | 2 h after |
| | After modeling | administration |
| Normal group | 11.72 | 11.55 |
| Model group | 5.45 | 5.63 |
| TRV-130: 0.5 mg/kg | 5.40 | 8.94 |
| Control compound 1: 75 mg/kg | 5.49 | 8.35 |
| Compound of the present invention | / | >8.50 |

It is shown from the experiment that the compound of the present invention has higher pain thresholds (PWT) at different doses (8 mg/kg, 25 mg/kg, and 75 mg/kg) than the control compound 1, which can inhibit pain well.

1. A compound shown in formula I 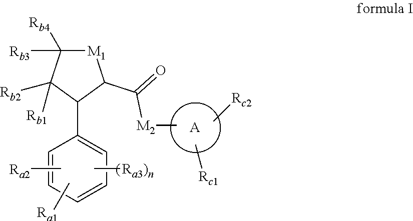
or a pharmaceutically acceptable salt or a stereoisomer thereof, wherein
Rₐ₁, Rₐ₂, and Ra are each independently selected from hydrogen, halogen, hydroxyl, C₁-C₅ alkyl, C₁-C₅ alkoxy, C₃-C₆ cycloalkyl, C₃-C₆ cycloalkoxy, or —N(R_{c3}R_{c4})ₘ, or any adjacent two of Rₐ₁, Rₐ₂, and Rₐ₃ are linked together to form a 5- to 7-membered ring, the 5- to 7-membered ring is a saturated or unsaturated carbocyclic or heterocyclic ring; the heterocyclic ring optionally contains one or more N, O, or S(═O)ₘ heteroatoms;
R_{b1}, R_{b2}, R_{b3}, and R_{b4} are each independently selected from hydrogen, halogen, hydroxyl, C₁-C₅ alkyl, or C₁-C₅ alkoxy; or any two of R_{b1}, R_{b2}, R_{b3}, and R_{b4} are linked together to form a 3- to 7-membered ring;
M₁ and M₂ are each independently selected from C, O, S(═O)ₘ, or N—Rₙ, where Rₙ is optionally selected from hydrogen or C₁-C₅ alkyl;
a ring A is A1 or A2: 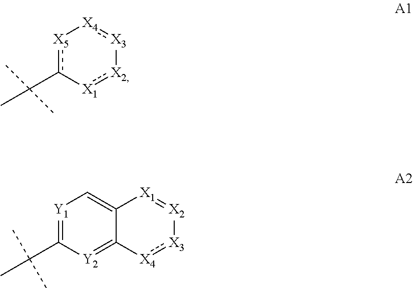 [symbol]
optionally represents a single bond or a double bond;
X₁, X₂, X₃, X₄, and X₅ are each independently selected from a bond, C, N, O, C═O, or S(═O)ₘ;
Y₁ and Y₂ are the same or different, and are each optionally selected from a C atom or a N atom;
R_{c1} and R_{c2} are each independently selected from hydrogen, halogen, cyano, hydroxyl, amino, C₁-C₅ alkyl, C₁-C₅ alkoxy, —S(═O)ₘ—R_{c3}R_{c4}, —C(═O)—NR_{c3}R_{c4}, —C(═S)—NR_{c3}R_{c4}, —N(R_{c3}R_{c4})ₘ, —P(═O)ₘ—R_{c3}R_{c4}, —C(═N)—NR_{c3}R_{c4}, —S(═O)₂NR_{c3}R_{c4}, —C₁-C₅ alkyl NR_{c3}R_{c4}, or —S(═O)(═NR_{c3})R_{c4};
R_{c3} and R_{c4} are each independently selected from hydrogen, hydroxyl, amino, C₁-C₅ alkyl, C₁-C₅ alkoxy, or a 3- to 7-membered ring, or R_{c3} and R_{c4} are linked together to form a 3- to 7-membered ring;
m and n are each optionally 0, 1, or 2; and
the alkyl, alkoxy, cycloalkyl, cycloalkoxy, 5- to 7-membered ring, or 3- to 7-membered ring is optionally substituted with one or more deuterium, halogen, hydroxyl, cyano, oxo, C₁-C₅ alkyl, C₁-C₅ alkoxy, or amino.

2. The compound shown in formula I according to claim 1, wherein Rₐ₁, Rₐ₂, and Rₐ₃ are each independently selected from fluorine, chlorine, methyl, ethyl, methoxy, ethoxy, propoxy, trifluoromethoxy, difluoromethoxy, or amino.

3. The compound shown in formula I according to claim 1, wherein any adjacent two of Rₐ₁, Rₐ₂, and Ra are linked together to form a 5- or 6-membered carbocyclic or heterocyclic ring.

4. The compound shown in formula I according to claim 1, wherein R_{b1}, R_{b2}, R_{b3}, and R_{b4} are each independently selected from hydrogen, methyl, trifluoromethyl, ethyl, methoxy, or ethoxy.

5. The compound shown in formula I according to claim 1, wherein any two of R_{b1}, R_{b2}, R_{b3}, and R_{b4} are linked together to form a 3-membered ring, a 4-membered ring, or a 5-membered ring.

6. The compound shown in formula I according to claim 1, wherein the ring A has the following structures:

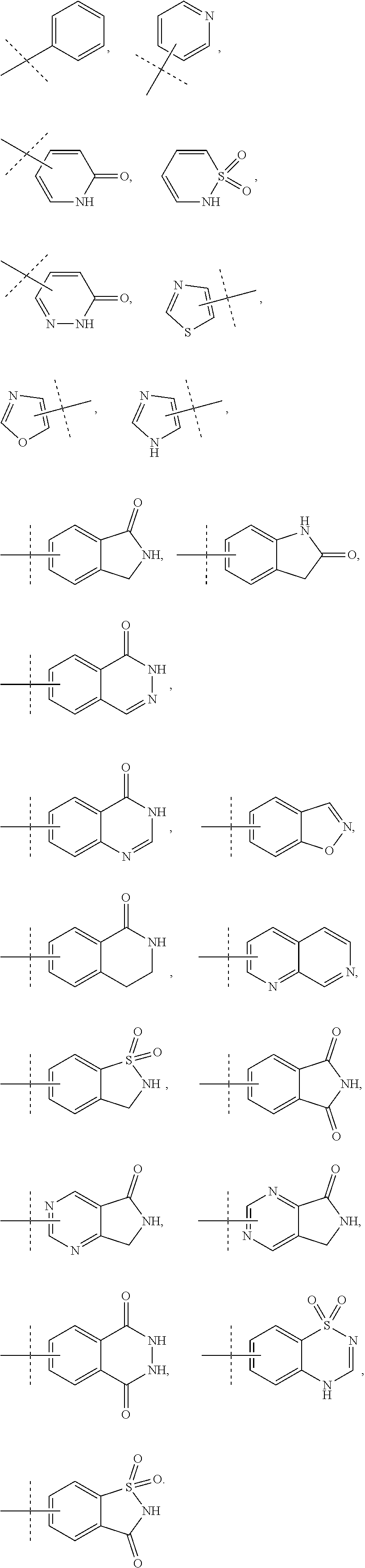

7. The compound shown in formula I according to claim 1, wherein the formula I has a structure shown in formula I-A or formula I-B:

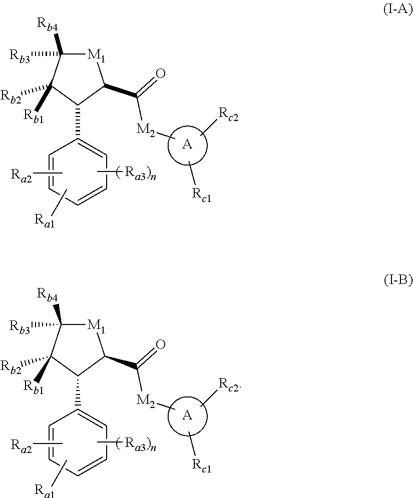

8. The compound shown in formula I according to claim 1, having the following structures:

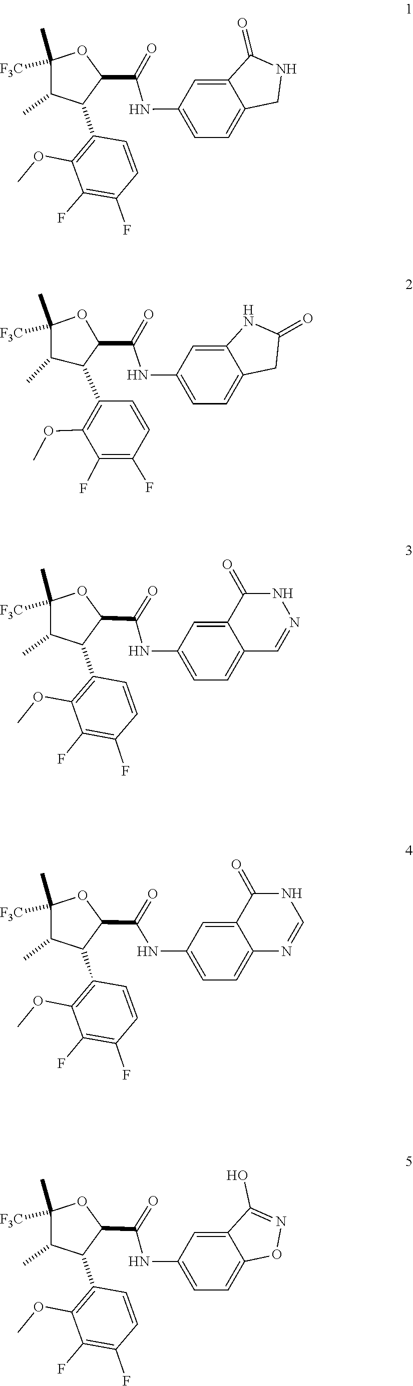

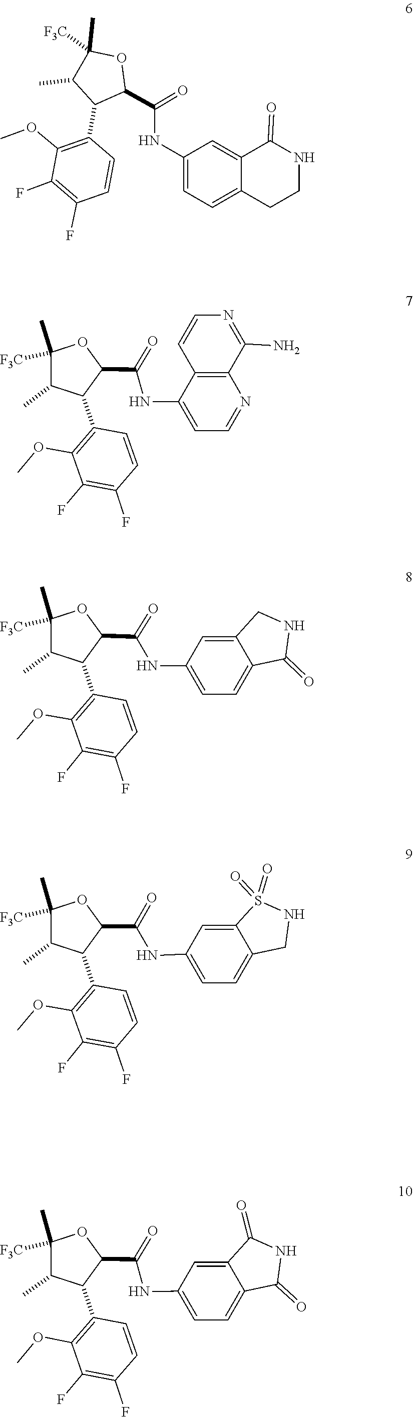 [SMILES: COc1c([C@H]2[C@H](C(=O)Nc3ccc4c(c3)C(=O)NCC4)O[C@@](C)(C(F)(F)F)[C@H]2C)ccc(F)c1F ; COc1c([C@H]2[C@H](C(=O)Nc3ccnc4c(N)nccc34)O[C@@](C)(C(F)(F)F)[C@H]2C)ccc(F)c1F ; COc1c([C@H]2[C@H](C(=O)Nc3ccc4c(c3)CNC4=O)O[C@@](C)(C(F)(F)F)[C@H]2C)ccc(F)c1F ; COc1c([C@H]2[C@H](C(=O)Nc3ccc4c(c3)S(=O)(=O)NC4)O[C@@](C)(C(F)(F)F)[C@H]2C)ccc(F)c1F ; COc1c([C@H]2[C@H](C(=O)Nc3ccc4c(c3)C(=O)NC4=O)O[C@@](C)(C(F)(F)F)[C@H]2C)ccc(F)c1F]

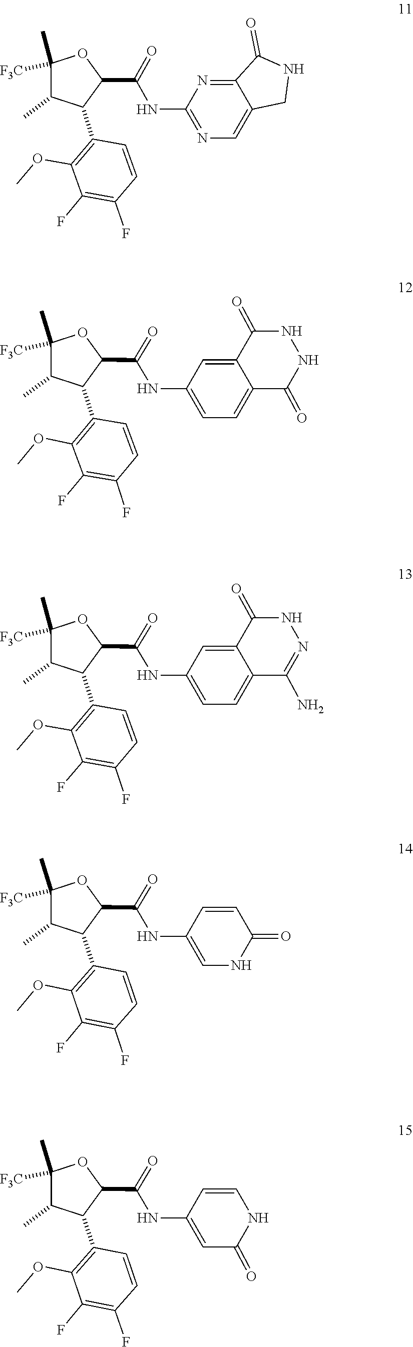 [SMILES: COc1c([C@H]2[C@H](C(=O)Nc3ncc4c(n3)C(=O)NC4)O[C@@](C)(C(F)(F)F)[C@H]2C)ccc(F)c1F ; COc1c([C@H]2[C@H](C(=O)Nc3ccc4c(=O)[nH][nH]c(=O)c4c3)O[C@@](C)(C(F)(F)F)[C@H]2C)ccc(F)c1F ; COc1c([C@H]2[C@H](C(=O)Nc3ccc4c(N)n[nH]c(=O)c4c3)O[C@@](C)(C(F)(F)F)[C@H]2C)ccc(F)c1F ; COc1c([C@H]2[C@H](C(=O)Nc3ccc(=O)[nH]c3)O[C@@](C)(C(F)(F)F)[C@H]2C)ccc(F)c1F ; COc1c([C@H]2[C@H](C(=O)Nc3cc[nH]c(=O)c3)O[C@@](C)(C(F)(F)F)[C@H]2C)ccc(F)c1F]

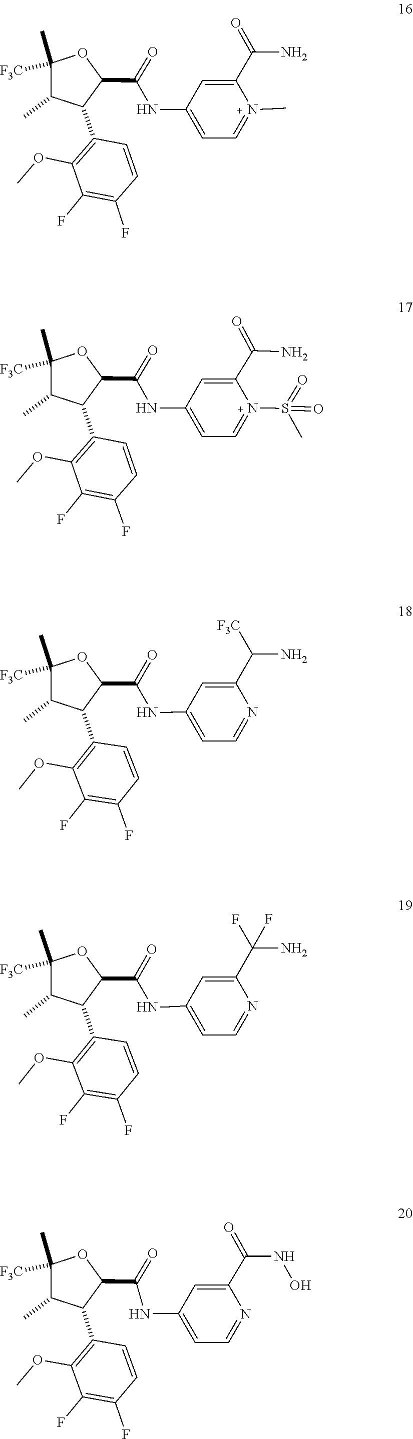

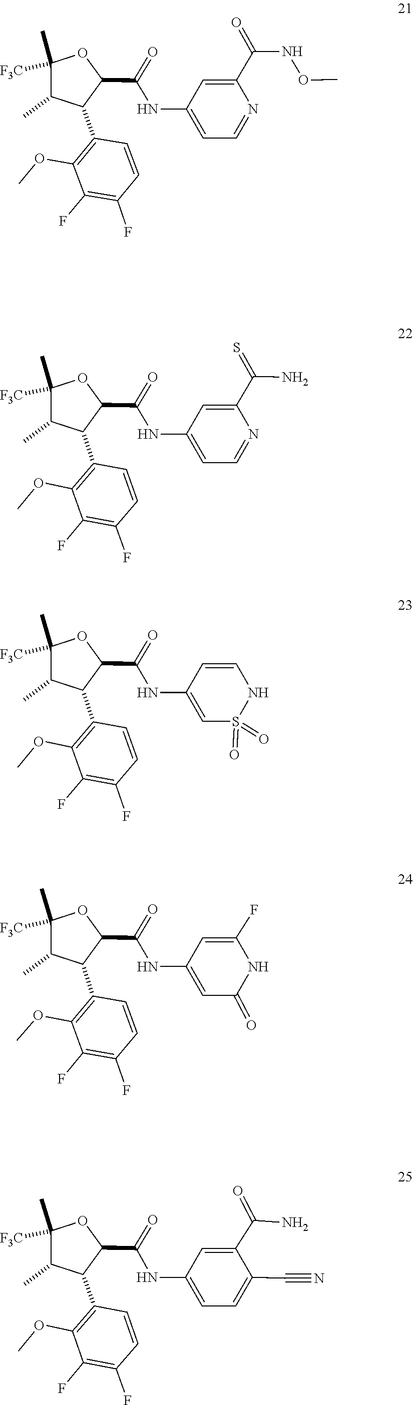

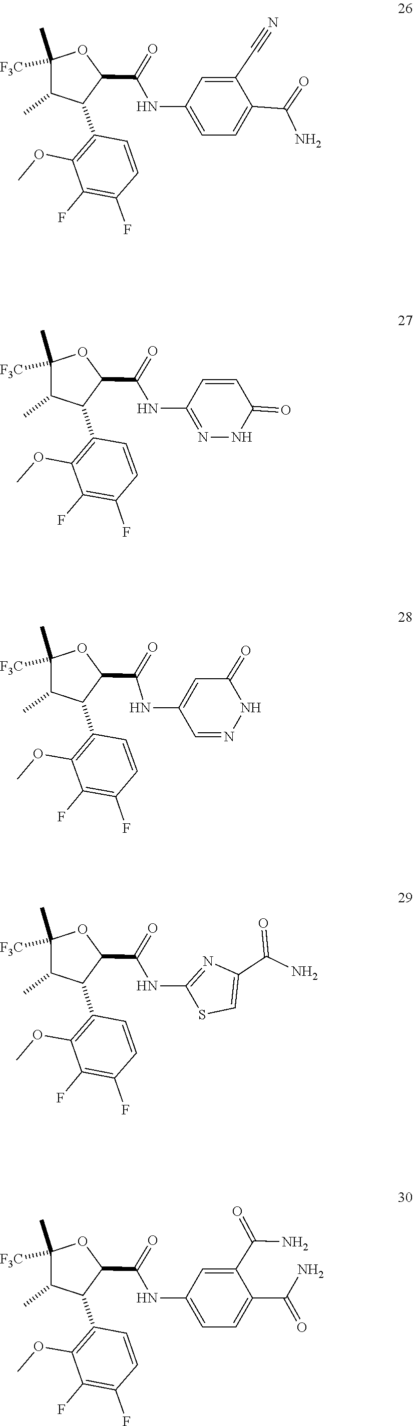 [SMILES: COc1c([C@H]2[C@H](C(=O)Nc3ccc(C(N)=O)c(C#N)c3)O[C@@](C)(C(F)(F)F)[C@H]2C)ccc(F)c1F ; COc1c([C@H]2[C@H](C(=O)Nc3ccc(=O)[nH]n3)O[C@@](C)(C(F)(F)F)[C@H]2C)ccc(F)c1F ; COc1c([C@H]2[C@H](C(=O)Nc3cn[nH]c(=O)c3)O[C@@](C)(C(F)(F)F)[C@H]2C)ccc(F)c1F ; COc1c([C@H]2[C@H](C(=O)Nc3nc(C(N)=O)cs3)O[C@@](C)(C(F)(F)F)[C@H]2C)ccc(F)c1F ; COc1c([C@H]2[C@H](C(=O)Nc3ccc(C(N)=O)c(C(N)=O)c3)O[C@@](C)(C(F)(F)F)[C@H]2C)ccc(F)c1F]

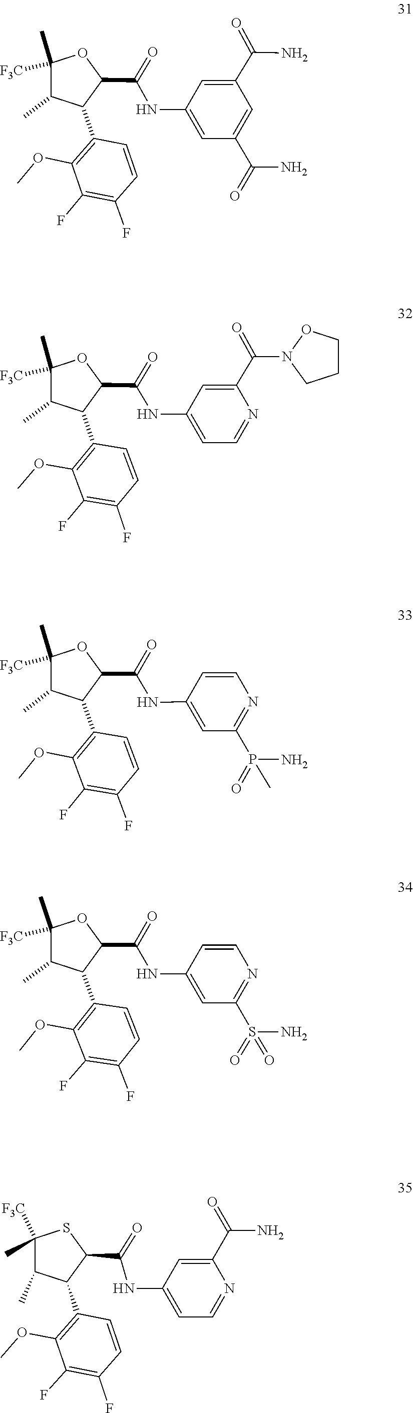 [SMILES: COc1c([C@H]2[C@H](C(=O)Nc3cc(C(N)=O)cc(C(N)=O)c3)O[C@@](C)(C(F)(F)F)[C@H]2C)ccc(F)c1F ; COc1c([C@H]2[C@H](C(=O)Nc3ccnc(C(=O)N4CCCO4)c3)O[C@@](C)(C(F)(F)F)[C@H]2C)ccc(F)c1F ; COc1c([C@H]2[C@H](C(=O)Nc3ccnc(P(C)(N)=O)c3)O[C@@](C)(C(F)(F)F)[C@H]2C)ccc(F)c1F ; COc1c([C@H]2[C@H](C(=O)Nc3ccnc(S(N)(=O)=O)c3)O[C@@](C)(C(F)(F)F)[C@H]2C)ccc(F)c1F ; COc1c([C@H]2[C@H](C(=O)Nc3ccnc(C(N)=O)c3)S[C@@](C)(C(F)(F)F)[C@H]2C)ccc(F)c1F]

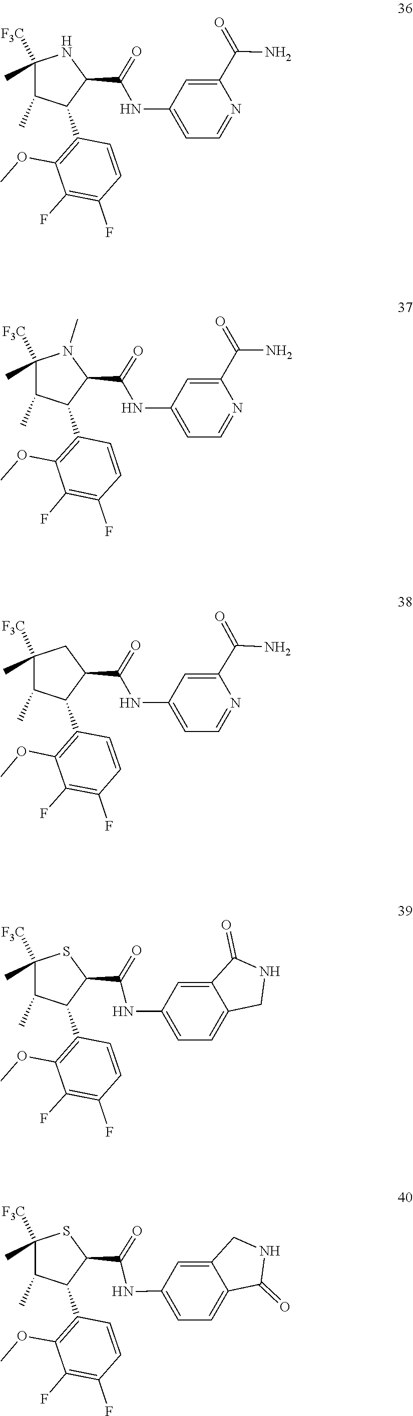 [SMILES: COc1c([C@H]2[C@H](C(=O)Nc3ccnc(C(N)=O)c3)N[C@@](C)(C(F)(F)F)[C@H]2C)ccc(F)c1F ; COc1c([C@H]2[C@H](C(=O)Nc3ccnc(C(N)=O)c3)N(C)[C@@](C)(C(F)(F)F)[C@H]2C)ccc(F)c1F ; COc1c([C@H]2[C@H](C(=O)Nc3ccnc(C(N)=O)c3)C[C@@](C)(C(F)(F)F)[C@H]2C)ccc(F)c1F ; COc1c([C@H]2[C@H](C(=O)Nc3ccc4c(c3)C(=O)NC4)S[C@@](C)(C(F)(F)F)[C@H]2C)ccc(F)c1F ; COc1c([C@H]2[C@H](C(=O)Nc3ccc4c(c3)CNC4=O)S[C@@](C)(C(F)(F)F)[C@H]2C)ccc(F)c1F]

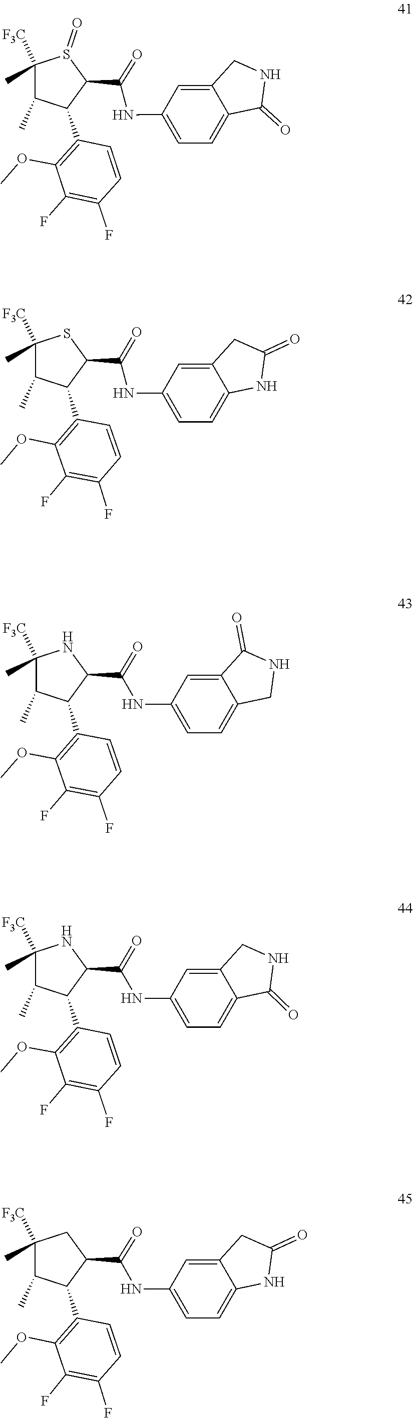 [SMILES: COc1c([C@H]2[C@H](C(=O)Nc3ccc4c(c3)CNC4=O)S(=O)[C@@](C)(C(F)(F)F)[C@H]2C)ccc(F)c1F ; COc1c([C@H]2[C@H](C(=O)Nc3ccc4c(c3)CC(=O)N4)S[C@@](C)(C(F)(F)F)[C@H]2C)ccc(F)c1F ; COc1c([C@H]2[C@H](C(=O)Nc3ccc4c(c3)C(=O)NC4)N[C@@](C)(C(F)(F)F)[C@H]2C)ccc(F)c1F ; COc1c([C@H]2[C@H](C(=O)Nc3ccc4c(c3)CNC4=O)N[C@@](C)(C(F)(F)F)[C@H]2C)ccc(F)c1F ; COc1c([C@H]2[C@H](C(=O)Nc3ccc4c(c3)CC(=O)N4)C[C@@](C)(C(F)(F)F)[C@H]2C)ccc(F)c1F]

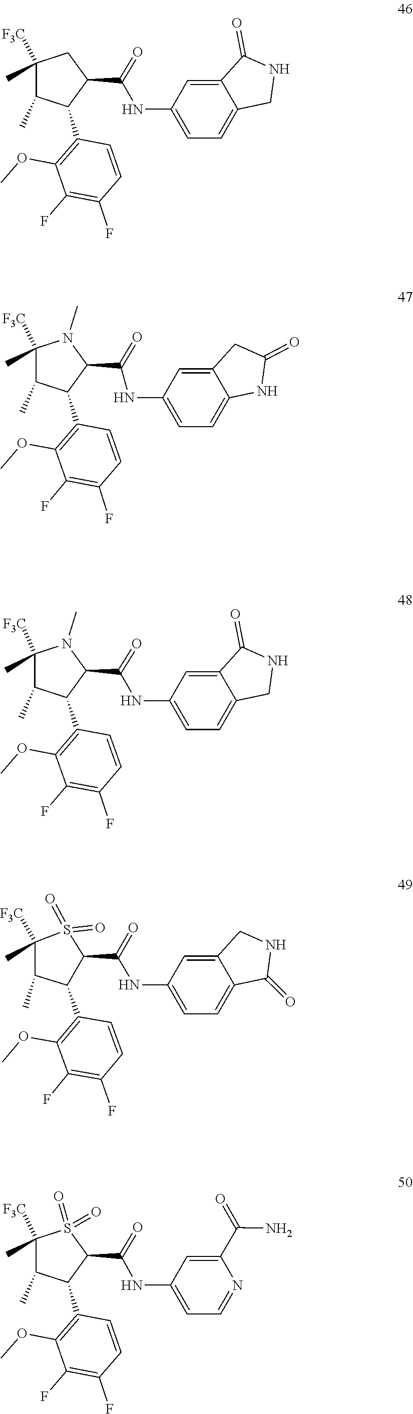

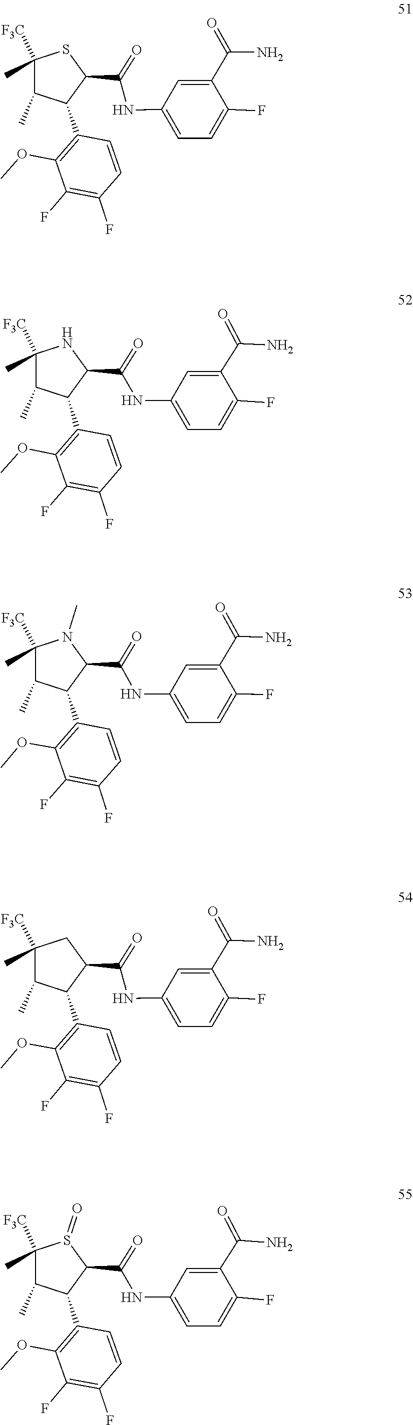 [SMILES: COc1c([C@H]2[C@H](C(=O)Nc3ccc(F)c(C(N)=O)c3)S[C@@](C)(C(F)(F)F)[C@H]2C)ccc(F)c1F ; COc1c([C@H]2[C@H](C(=O)Nc3ccc(F)c(C(N)=O)c3)N[C@@](C)(C(F)(F)F)[C@H]2C)ccc(F)c1F ; COc1c([C@H]2[C@H](C(=O)Nc3ccc(F)c(C(N)=O)c3)N(C)[C@@](C)(C(F)(F)F)[C@H]2C)ccc(F)c1F ; COc1c([C@H]2[C@H](C(=O)Nc3ccc(F)c(C(N)=O)c3)C[C@@](C)(C(F)(F)F)[C@H]2C)ccc(F)c1F ; COc1c([C@H]2[C@H](C(=O)Nc3ccc(F)c(C(N)=O)c3)S(=O)[C@@](C)(C(F)(F)F)[C@H]2C)ccc(F)c1F]

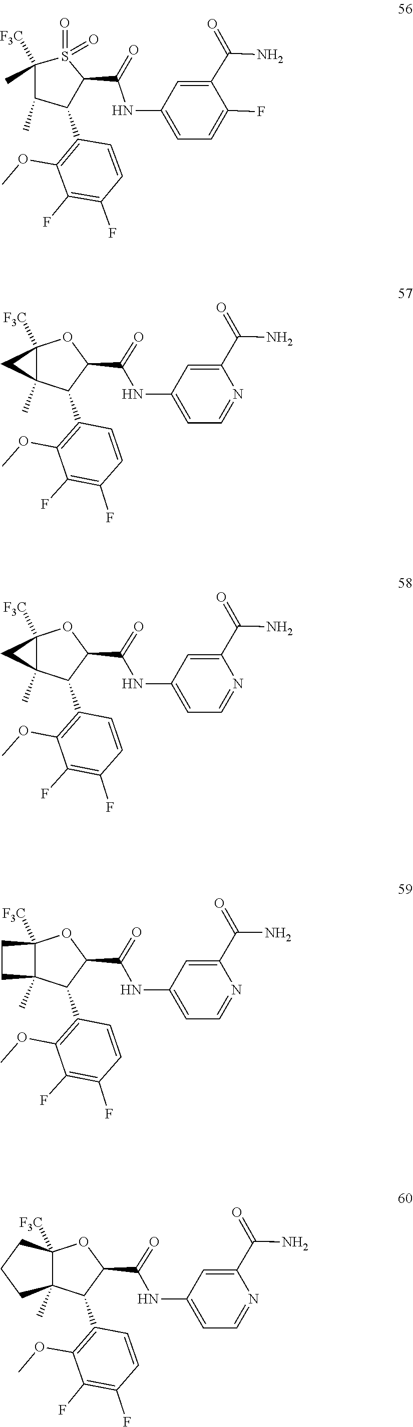

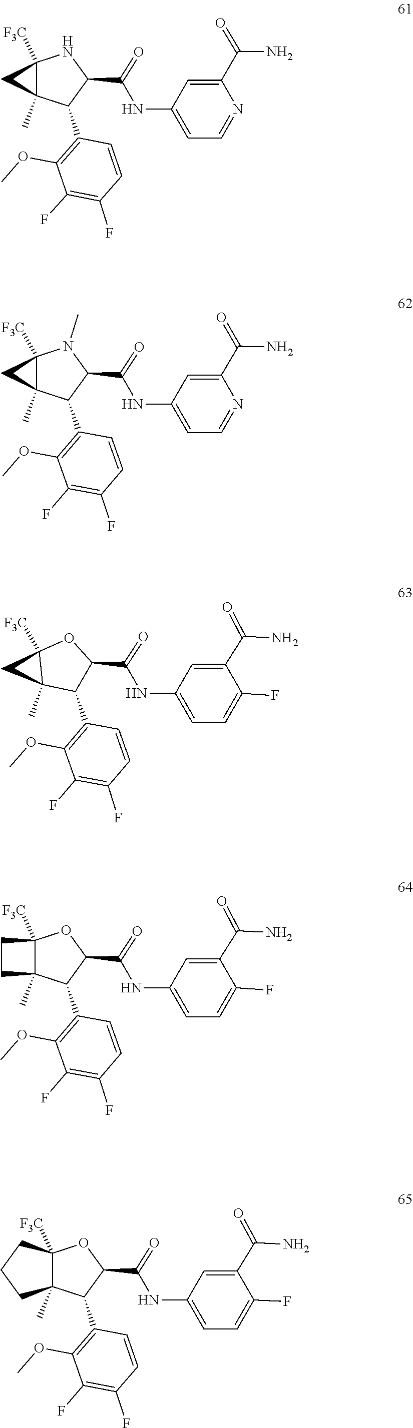 [SMILES: COc1c([C@H]2[C@H](C(=O)Nc3ccnc(C(N)=O)c3)N[C@]3(C(F)(F)F)C[C@]23C)ccc(F)c1F ; COc1c([C@H]2[C@H](C(=O)Nc3ccnc(C(N)=O)c3)N(C)[C@]3(C(F)(F)F)C[C@]23C)ccc(F)c1F ; COc1c([C@H]2[C@H](C(=O)Nc3ccc(F)c(C(N)=O)c3)O[C@]3(C(F)(F)F)C[C@]23C)ccc(F)c1F ; COc1c([C@H]2[C@H](C(=O)Nc3ccc(F)c(C(N)=O)c3)O[C@]3(C(F)(F)F)CC[C@]23C)ccc(F)c1F ; COc1c([C@H]2[C@H](C(=O)Nc3ccc(F)c(C(N)=O)c3)O[C@]3(C(F)(F)F)CCC[C@]23C)ccc(F)c1F]

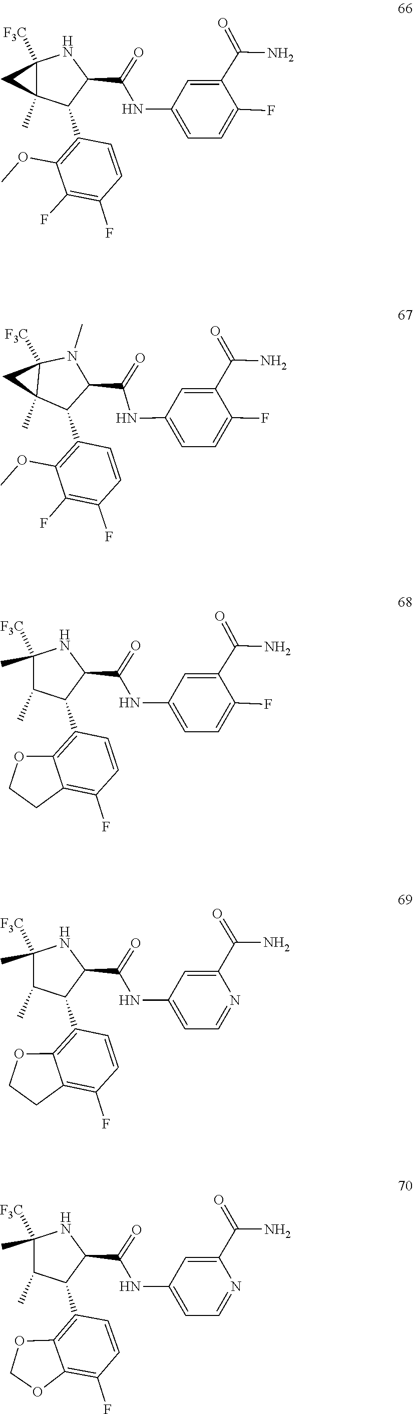 [SMILES: COc1c([C@H]2[C@H](C(=O)Nc3ccc(F)c(C(N)=O)c3)N[C@]3(C(F)(F)F)C[C@]23C)ccc(F)c1F ; COc1c([C@H]2[C@H](C(=O)Nc3ccc(F)c(C(N)=O)c3)N(C)[C@]3(C(F)(F)F)C[C@]23C)ccc(F)c1F ; C[C@H]1[C@@H](c2ccc(F)c3c2OCC3)[C@H](C(=O)Nc2ccc(F)c(C(N)=O)c2)N[C@@]1(C)C(F)(F)F ; C[C@H]1[C@@H](c2ccc(F)c3c2OCC3)[C@H](C(=O)Nc2ccnc(C(N)=O)c2)N[C@@]1(C)C(F)(F)F ; C[C@H]1[C@@H](c2ccc(F)c3c2OCO3)[C@H](C(=O)Nc2ccnc(C(N)=O)c2)N[C@@]1(C)C(F)(F)F]

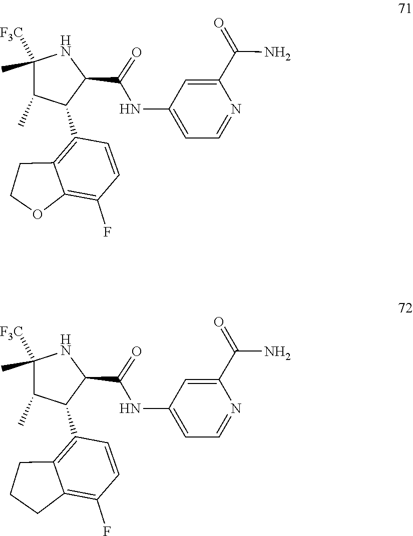 [SMILES: C[C@H]1[C@@H](c2ccc(F)c3c2CCO3)[C@H](C(=O)Nc2ccnc(C(N)=O)c2)N[C@@]1(C)C(F)(F)F ; C[C@H]1[C@@H](c2ccc(F)c3c2CCC3)[C@H](C(=O)Nc2ccnc(C(N)=O)c2)N[C@@]1(C)C(F)(F)F]

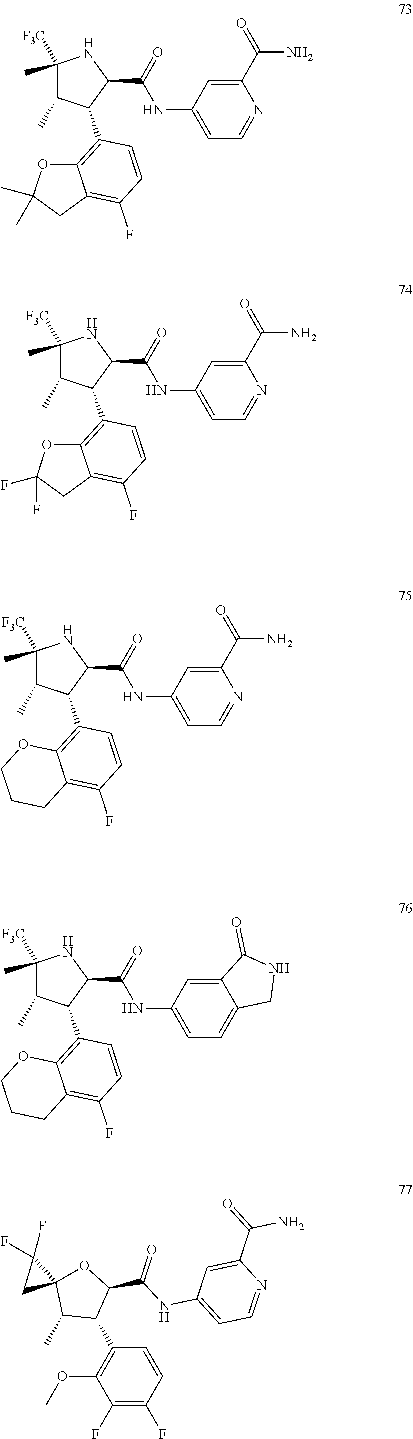 [SMILES: C[C@H]1[C@@H](c2ccc(F)c3c2OC(C)(C)C3)[C@H](C(=O)Nc2ccnc(C(N)=O)c2)N[C@@]1(C)C(F)(F)F ; C[C@H]1[C@@H](c2ccc(F)c3c2OC(F)(F)C3)[C@H](C(=O)Nc2ccnc(C(N)=O)c2)N[C@@]1(C)C(F)(F)F ; C[C@H]1[C@@H](c2ccc(F)c3c2OCCC3)[C@H](C(=O)Nc2ccnc(C(N)=O)c2)N[C@@]1(C)C(F)(F)F ; C[C@H]1[C@@H](c2ccc(F)c3c2OCCC3)[C@H](C(=O)Nc2ccc3c(c2)C(=O)NC3)N[C@@]1(C)C(F)(F)F ; COc1c([C@H]2[C@H](C(=O)Nc3ccnc(C(N)=O)c3)O[C@]3(CC3(F)F)[C@H]2C)ccc(F)c1F]

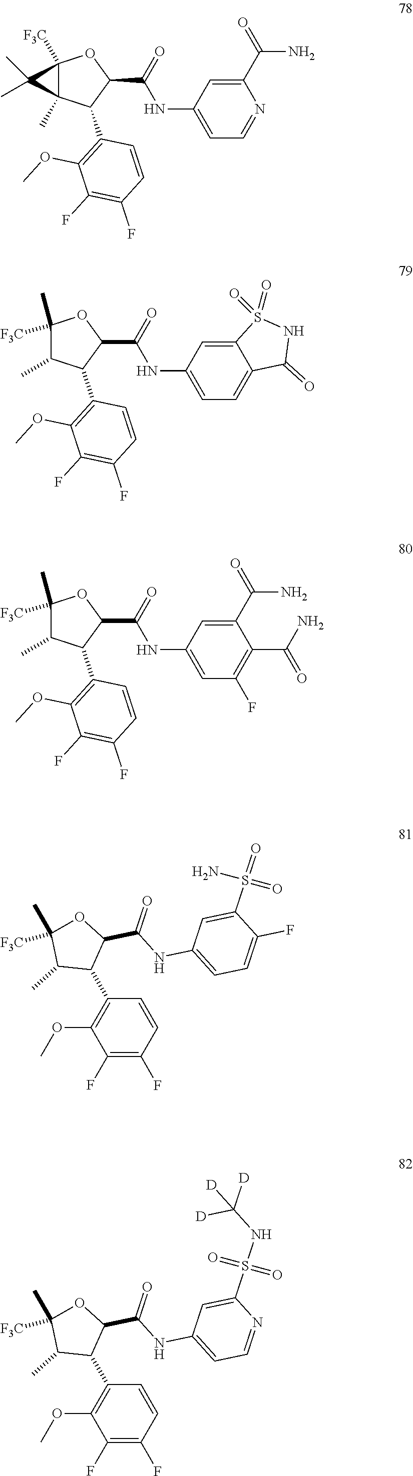

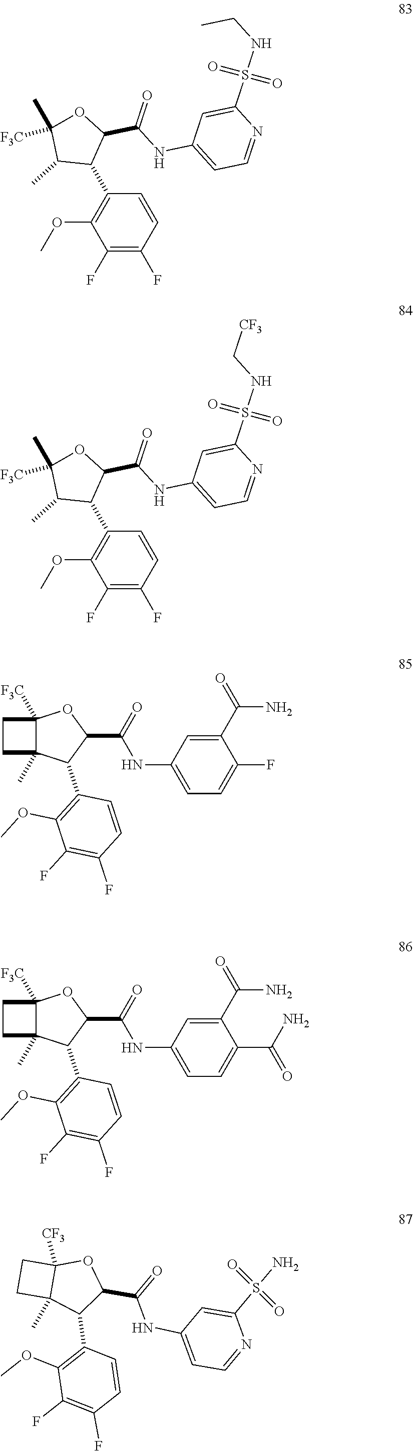

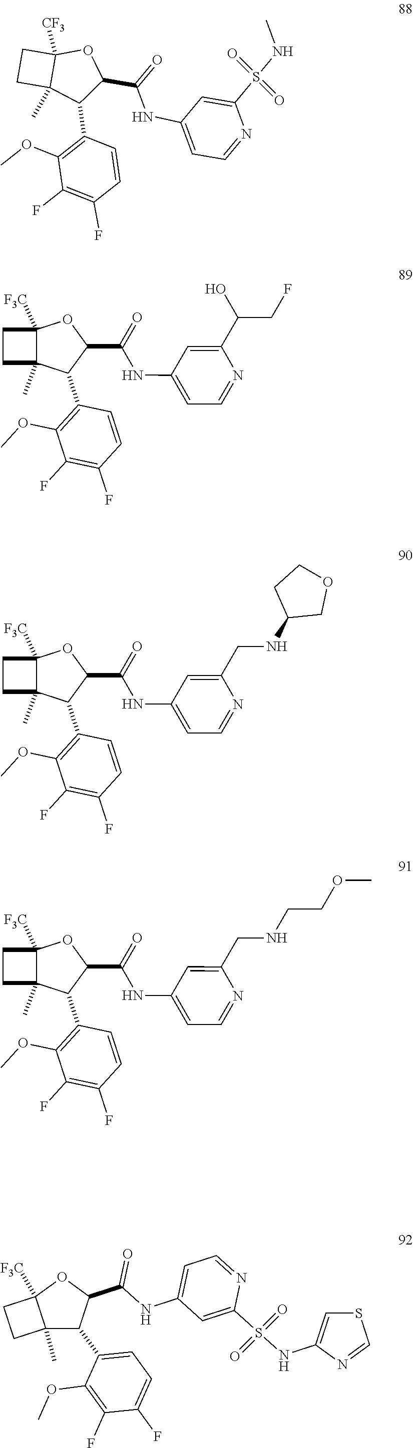

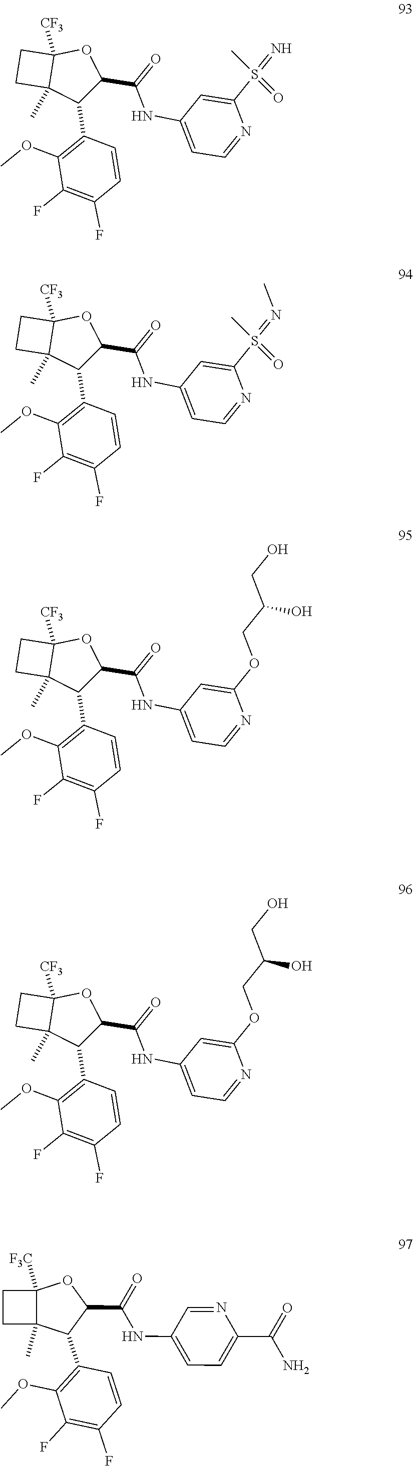

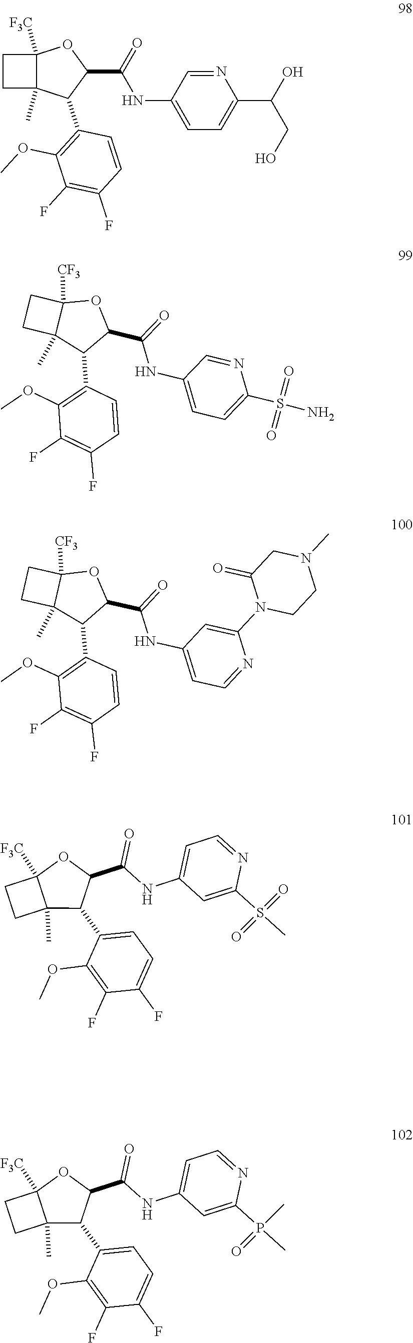

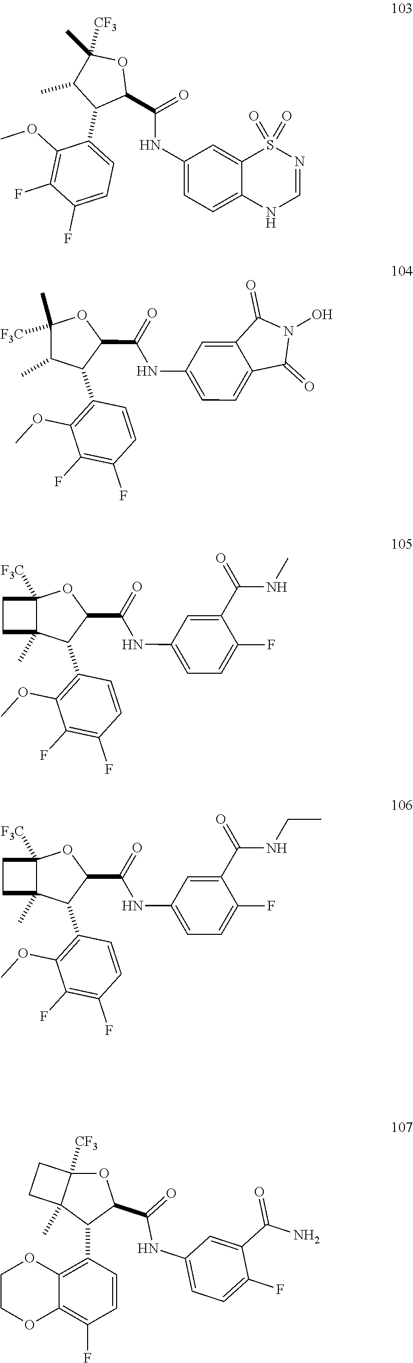

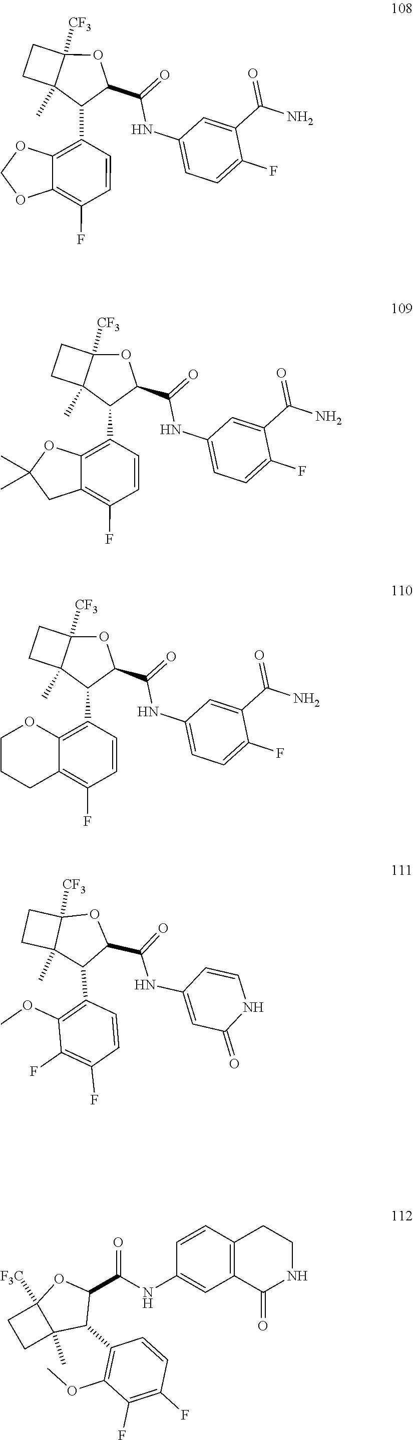

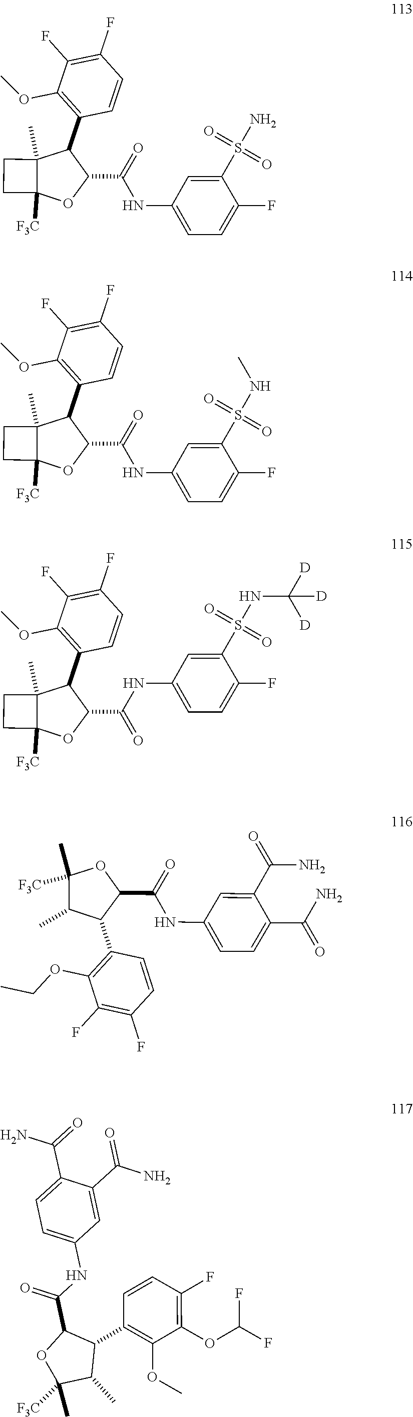

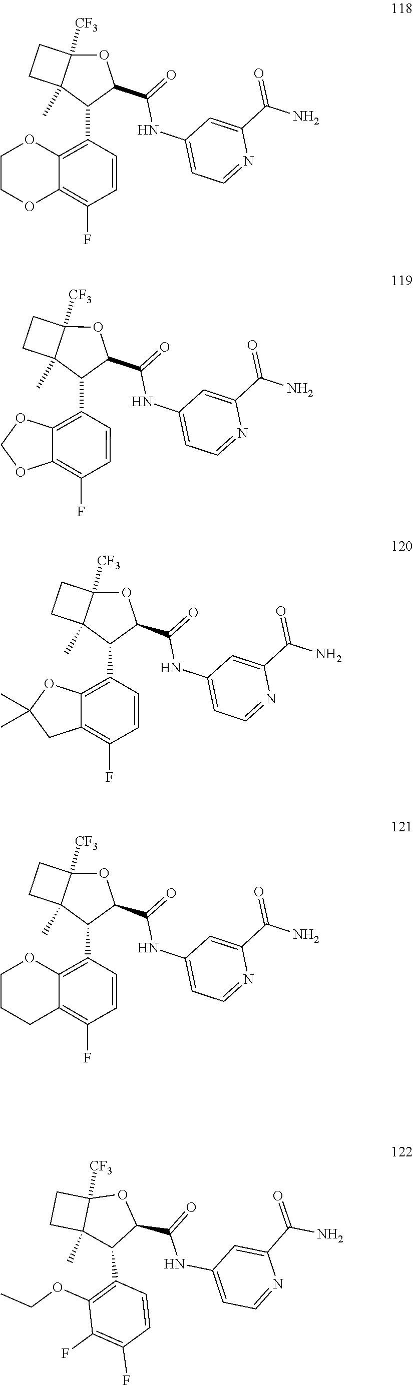

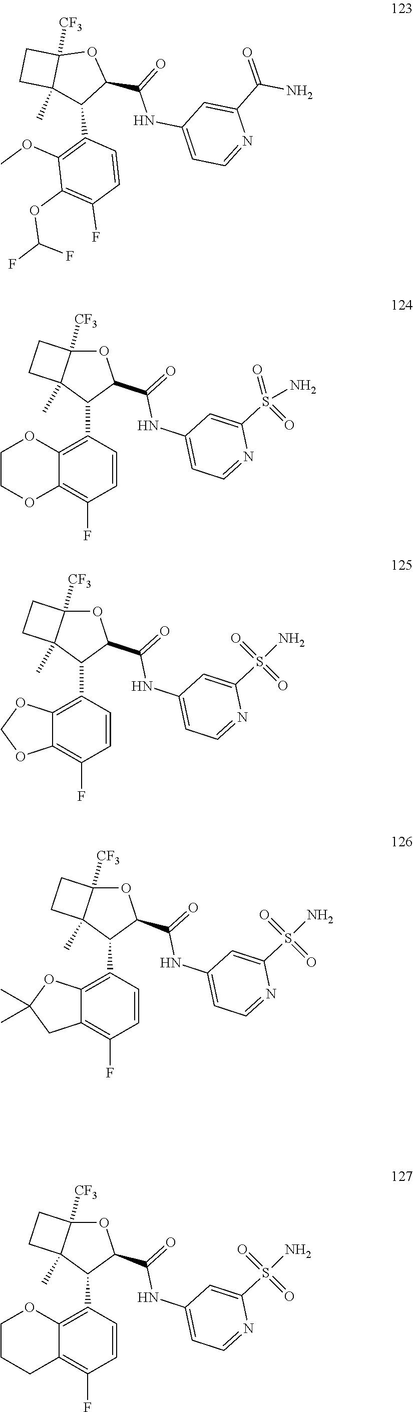

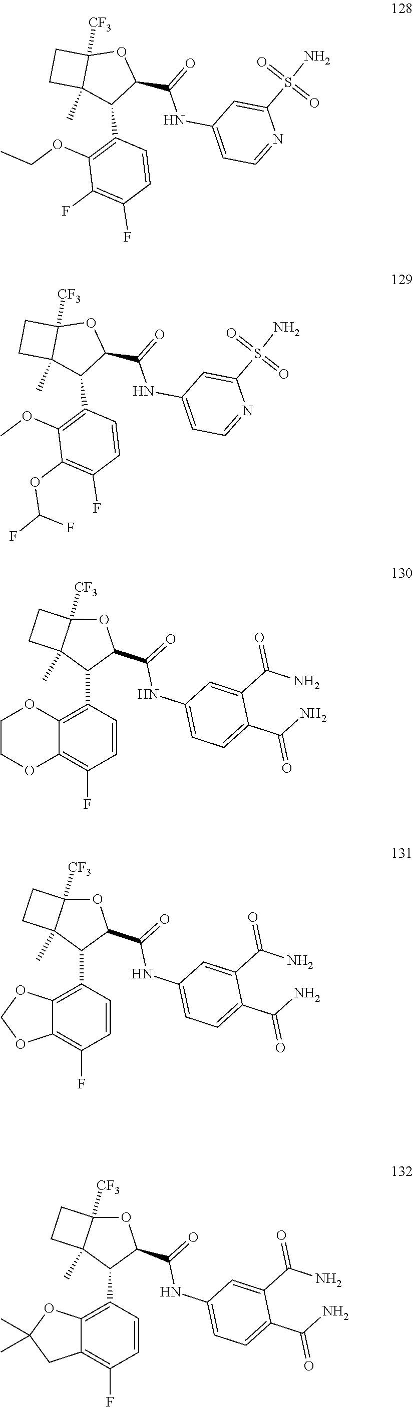

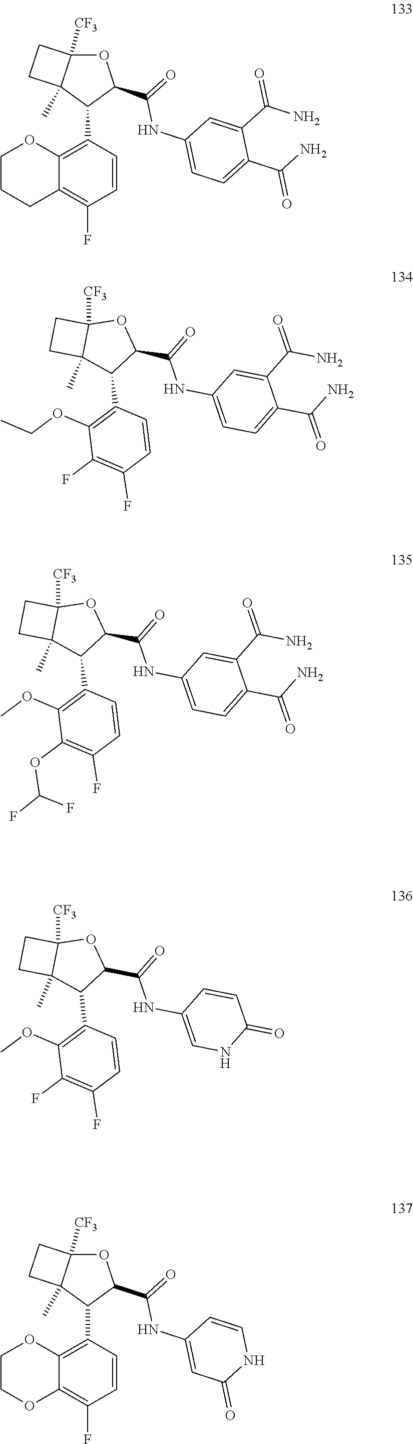

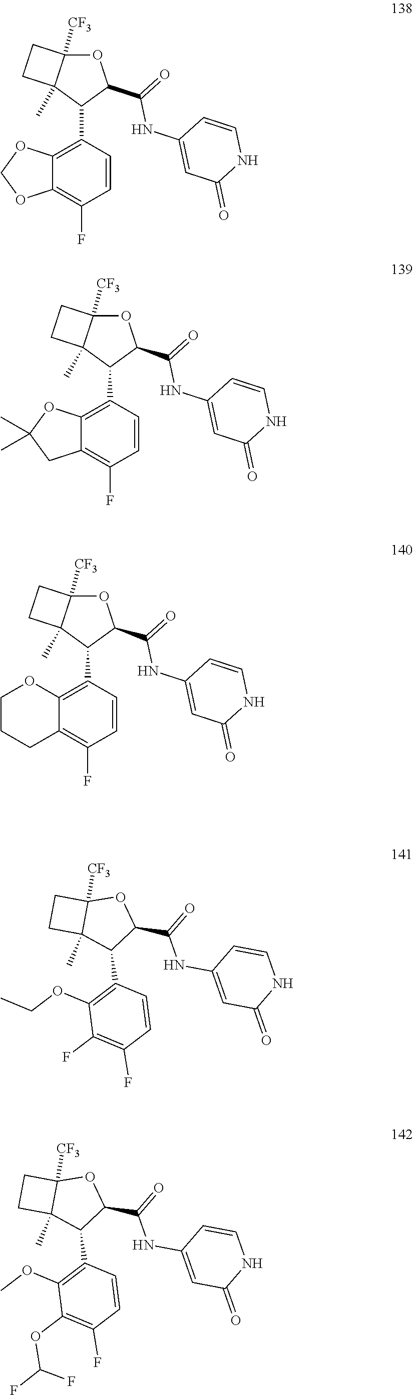

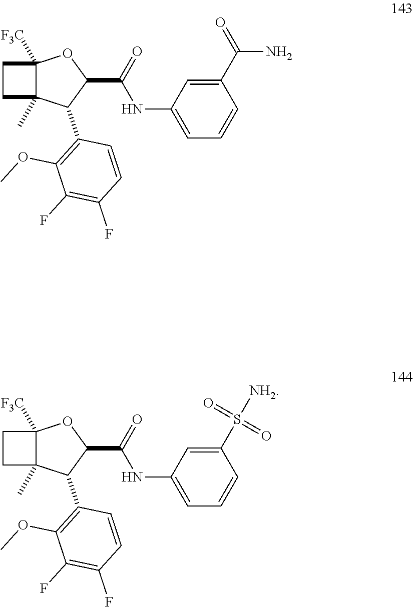

9. A pharmaceutical composition, wherein the composition contains the compound shown in formula I according to claim 1 or a pharmaceutically acceptable salt or a stereoisomer thereof, and a pharmaceutically acceptable excipient.

10. A method for the treatment, prevention, or relief of a disease related to voltage-gated sodium channels in a subject in need thereof, wherein comprising administering a therapeutically effective amount the compound shown in formula I according to claim 1 or a pharmaceutically acceptable salt or a stereoisomer thereof.

11. The method according to claim 10, wherein the disease is pain, multiple sclerosis, Charcot-Marie-Tooth disease, incontinence, pathologic coughs, or arrhythmia.

12. A pharmaceutical composition, wherein the composition contains the compound shown in formula I according to claim 8 or a pharmaceutically acceptable salt or a stereoisomer thereof, and a pharmaceutically acceptable excipient.

13. A method for the treatment, prevention, or relief of a disease related to voltage-gated sodium channels in a subject in need thereof, wherein comprising administering a therapeutically effective amount the pharmaceutical composition according to claim 12 or a pharmaceutically acceptable salt or a stereoisomer thereof.

14. The method according to claim 13, wherein the disease is pain, multiple sclerosis, Charcot-Marie-Tooth disease, incontinence, pathologic coughs, or arrhythmia.

15. A method for the treatment, prevention, or relief of a disease related to voltage-gated sodium channels in a subject in need thereof, wherein comprising administering a therapeutically effective amount the pharmaceutical composition according to claim 9 or a pharmaceutically acceptable salt or a stereoisomer thereof.

16. The method according to claim 15, wherein the disease is pain, multiple sclerosis, Charcot-Marie-Tooth disease, incontinence, pathologic coughs, or arrhythmia.
